(19) United States
(12) Patent Application Publication
(10) Pub. No.: US 2025/0025417 A1
(43) Pub. Date: Jan. 23, 2025

(54) ORAL ABIRATERONE FORMULATIONS

(71) Applicant: PROPELLA THERAPEUTICS, INC., Pittsboro, NC (US)

(72) Inventors: Matthew J. SHARP, Pittsboro, NC (US); William R. MOORE, Pittsboro, NC (US)

(21) Appl. No.: 18/689,500

(22) PCT Filed: Sep. 7, 2022
(86) PCT No.: PCT/US2022/042701

§ 371 Date: Mar. 6, 2024

Related U.S. Application Data
(60) Provisional application No. 63/241,705, filed on Sep. 8, 2021.

(51) Int. Cl. A61K 9/107; A61K 31/58; A61K 45/06; A61K 47/14

(52) U.S. Cl. CPC A61K 9/107; A61K 31/58; A61K 45/06; A61K 47/14

(57) ABSTRACT

Provided herein are oral abiraterone prodrug formulations, related methods and kits, for example, for oral administration to a subject having a sex hormone-dependent benign or malignant disorder such as prostate cancer, an androgen receptor driven cancer, a syndrome due to androgen excess, and/or a syndrome due to glucocorticoid excess such as hypercortisolemia.

The present disclosure relates generally to novel oral formulations of abiraterone prodrugs. The disclosure is subject to a wide range of applications, such as for administration to a patient suffering from an androgen or estrogen hormone-dependent benign or malignant disorder or androgen receptor driven cancer, including various cancers (such as prostate cancer, bladder cancer, hepatocellular carcinoma, lung cancer, breast cancer, endometrial cancer, and ovarian cancer, etc.), and for the treatment of non-oncologic syndromes due to the overproduction of androgens (including both classical and nonclassical congenital adrenal hyperplasia, endometriosis, polycystic ovary syndrome, precocious puberty, hirsutism, etc.) or due to the overproduction of glucocorticoids, typically cortisol in conditions such as Cushing's syndrome or Cushing's disease.

### BACKGROUND

Abiraterone ((3β)-17-(pyridin-3-yl)androsta-5,16-dien-3-ol; CAS #: 154229-19-3); Formula: C₂₄H₃₁NO; Mol. Weight: 349.5 g/mol) is an inhibitor of CYP17A1 (which is a member of the cytochrome P450 superfamily of enzymes that catalyze the synthesis of cholesterol, steroids and other lipids and are involved in drug metabolism). CYP17A1 has both 17α-hydroxylase activity and 17,20-lyase activity. Abiraterone potently and selectively inhibits both CYP17A1 17α-hydroxylase and 17,20-lyase enzyme activities. The 17α-hydroxylase activity of CYP17A1 is required for the generation of glucocorticoids such as cortisol. However, both the hydroxylase and 17,20-lyase activities of CYP17A1 are required for the production of androgenic (e.g., androstenedione, testosterone, and dihyrotestosterone) and estrogenic (estrone, estradiol. estratriol) steroids through the conversion of 17α-hydroxypregnenolone to the sex steroid precursor, dehydroepiandrosterone, see FIG. 1. Thus, abiraterone interferes with the synthesis of androgens and estrogens in the gonads (primarily in the testes and overies) and extra-gonadally (e.g., in the adrenals and in the tumors themselves).

Though abiraterone itself is poorly absorbed, it can be administered orally as an abiraterone acetate prodrug. Abiraterone acetate is also poorly absorbed, but can be converted to abiraterone in the gut, which is poorly absorbed into the bloodstream following the cleavage of the acetate prodrug. Abiraterone acetate ((3β)-17-(3-Pyridyl)androsta-5, acetate; CAS #154229-18-2) is approved in the United States for treatment of castration resistant or castration sensitive prostate cancer under the brand name Zytiga®. Abiraterone acetate is now also available globally.

It is known that orally administered abiraterone acetate prodrug is not significantly absorbed by the gastrointestinal tract (and little prodrug can be detected in blood plasma). Instead, it has been shown that abiraterone acetate is hydrolyzed to abiraterone in the intraluminal environment resulting in generation of abiraterone supersaturation, which is responsible for creating the strong driving force for abiraterone absorption (Stappaerts et al., Eur. J. Pharmaceutics Biopharmaceutics 90:1, 2015).

Because abiraterone blocks the normal physiologic production of steroids by the adrenal glands, its prodrug formulation is commonly prescribed with administration of a low dose of a steroid to prevent adrenal insufficiency. Indeed, Zytiga® (250 mg tablets) is approved in the United States only in combination with prednisone for the treatment of patients with metastatic castration resistant prostate cancer (CRPC) and patients with metastatic castration-sensitive prostate cancer (CSPC). The prescribing information provided with Zytiga® recommends oral administration of 1,000 mg (4×250 mg tablets) once daily in combination with prednisone (5 mg) administered orally twice daily for CRPC patients or once daily for CSPC patients. In Europe, the use of Zytiga® is approved only in combination with either prednisone or prednisolone.

Because the administration of abiraterone acetate with food increases the absorption of abiraterone acetate (and, therefore, has the potential to result in increased and highly variable exposures, which can potentially cause various side effects including cardiovascular side effects and/or hepatotoxicity etc., the prodrug should be consumed on an empty stomach at least one hour before, or two hours after, a meal. Indeed, the prescribing information for Zytiga® states it must be taken on an empty stomach, and no food should be consumed for at least two hours before oral dosing and at least one hour after oral dosing.

The prescribing information explains that for a daily oral dose of 1,000 mg of Zytiga© in patients with metastatic CRPC, abiraterone's steady-state Cₘₐₓ values were 226±178 ng/mL (mean±SD) and its area under the curve (AUC) values were 1173±690 ng·hr/mL (mean±SD). A single-dose (1,000 mg) cross-over study of Zytiga® in healthy subjects found that systemic exposure of abiraterone was increased when Zytiga® was administered with food. Specifically, abiraterone's Cₘₐₓ and AUC values were approximately 7- and 5-fold higher, respectively, when Zytiga® was administered with a low-fat meal (7% fat, 300 calories) and approximately 17- and 10-fold higher, respectively, when Zytiga® was administered with a high-fat meal (57% fat, 825 calories).

The currently approved solid dosage oral form of the prodrug abiraterone acetate has several disadvantages. For example, it has very low bioavailability that necessitates a large daily pill burden for patients (4×250 mg tablets once daily). In addition, it causes highly variable blood levels in patients due to the combination of low bioavailability and a large food effect. Further, as abiraterone is rapidly cleared, this approved dosing regimen results in a daily Cₘᵢₙ of abiraterone, which is believed to be associated with a loss of therapeutic effect in metastatic CRPC patients.

### SUMMARY

The present disclosure generally relates to novel oral abiraterone prodrugs formulations, and methods of using the same, for example, in treating a subject having a sex hormone-dependent benign or malignant disorder, an androgen receptor driven cancer, and/or a syndrome due to androgen and/or glucocorticoid excess.

U.S. Pat. No. 10,792,292 B2, issued to Propella Therapeutics, Inc. on Oct. 6, 2020 and U.S. Provisional Application Nos. 63/073,502, filed Sep. 2, 2020, and 63/149,550, filed Feb. 15, 2021, describe useful abiraterone prodrugs, in particular, abiraterone decanoate, as a breakthrough over the currently marketed Zytiga® tablets in that they provide increased bioavailability, elimination of the food effect, reduced pill burden, less frequent dosing frequency, and sustained effective blood plasma levels of abiraterone, e.g., continuous plasma exposures above daily Cₘᵢₙ levels observed for oral administration of abiraterone acetate, for example, for at least one week, typically, for at least two weeks and up to ten weeks or more following administration of the abiraterone prodrug formulation. It was demonstrated that the novel abiraterone prodrugs and formulations therein are suitable for dosing once a week, once a month, once every two months, once every three months, or even less frequently, for treating a subject having a sex hormone-dependent benign or malignant disorder, an androgen receptor driven cancer, a syndrome due to androgen excess and/or a syndrome due to glucocorticoid excess.

The present disclosure is based, in part, on that with proper lipid-based drug delivery system, abiraterone decanoate can be orally bioavailable with good to excellent oral bioavailability based on plasma abiraterone concentrations, which can achieve an effective plasma concentration of abiraterone for modulating serum steroid level, such as reduction of serum androgen levels. With enhanced bioavailability, the oral abiraterone decanoate formulations herein can be suited for dosing frequencies ranging from once a day to once a week, such as once a day or once every two or three days. As shown herein, a single oral administration of the pharmaceutical composition herein can achieve a sustained inhibition of CYP17A1 for a period of 24 hours or more. This disclosure thus provides alternative methods for administering abiraterone prodrugs, such as abiraterone lipophilic esters, in particular, abiraterone decanoate, to treat various diseases or disorders described herein, such as a prostate cancer described herein, which can also be advantageous in comparison with the currently marketed Zytiga® tablets.

In some embodiments, the present disclosure provides the following exemplary embodiments:
[1] A pharmaceutical composition comprising: (a) abiraterone decanoate; and (b) a lipid-based drug delivery system, wherein abiraterone decanoate has the following structure:

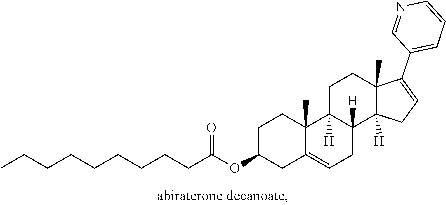

wherein the pharmaceutical composition is formulated for oral delivery of abiraterone decanoate.
[2] The pharmaceutical composition of [1], wherein the lipid-based drug delivery system comprises: (1) a triglyceride, monoglyceride, diglyceride, and/or a propylene glycol ester; and (2) a surfactant comprising a polyglyceryl ester and/or polyoxyglyceride.
[3] The pharmaceutical composition of [1] or [2], wherein the lipid-based drug delivery system comprises a triglyceride.
[4] The pharmaceutical composition of [1] or [2], wherein the lipid-based drug delivery system comprises a medium-chain triglyceride (e.g., Labrafac™ lipophile WL 1349, or medium-chain triglycerides of caprylic (C8) and capric (C10) acids).
[5] The pharmaceutical composition of any one of [1]-[4], wherein the lipid-based drug delivery system comprises a monoglyceride and/or diglyceride.
[6] The pharmaceutical composition of any one of [1]-[4], wherein the lipid-based drug delivery system comprises a glycerol/glyceryl linoleate (e.g., Maisine® CC, mono-, di- and triglycerides of mainly linoleic (C_{18:2}) and oleic (C_{18:1}) acids, the diester fraction being predominant).
[7] The pharmaceutical composition of any one of [1]-[6], wherein the lipid-based drug delivery system comprises a propylene glycol ester.
[8] The pharmaceutical composition of any one of [1]-[6], wherein the lipid-based drug delivery system comprises propylene glycol monocaprylate (e.g., Capmul PG-8) and/or propylene glycol monolaurate (e.g., Capmul PG-12, or Lauroglycol™ 90).
[9] The pharmaceutical composition of any one of [1]-[8], wherein the lipid-based drug delivery system comprises a surfactant comprising a polyglycerol ester.
[10] The pharmaceutical composition of any one of [1]-[8], wherein the lipid-based drug delivery system comprises a surfactant comprising polyglyceryl oleate (e.g., Plurol Oleique CC 497 (polyglyceryl-3 dioleate)).
[11] The pharmaceutical composition of any one of [1]-[10], wherein the lipid-based drug delivery system comprises a surfactant comprising a polyoxyglyceride.
[12] The pharmaceutical composition of any one of [1]-[10], wherein the lipid-based drug delivery system comprises a surfactant comprising macrogolglycerol hydroxystearate (e.g., Kolliphor RH 40), oleoyl polyoxyl-6 glycerides (e.g., Labrafil® M 1944 CS) or lauroyl polyoxyl-6 glycerides (e.g., Labrafil 2130).
[13] The pharmaceutical composition of any one of [1]-[12], wherein the abiraterone decanoate is dispersed, such as homogeneously dispersed or dissolved, in the lipid-based drug delivery system, with a concentration ranging from about 1 mg/g to about 250 mg/g, e.g., about 20 mg/g to about 150 mg/g.
[14] The pharmaceutical composition of any one of [1]-[13], formulated in the form of an oral dosage form, such as a capsule (e.g., a soft gel capsule).
[15] The pharmaceutical composition of any one of [1]-[14], which is characterized by one or more of the following: (1) the pharmaceutical composition is storage stable at room temperature; (2) the recovery of abiraterone decanoate is greater than 50% when the pharmaceutical composition is assessed using an in vitro dispersion test; and (3) upon oral administration to a mammal, the pharmaceutical composition is capable of delivering a sufficient amount of abiraterone decanoate to the mammal to achieve a therapeutically effective plasma concentration of abiraterone, e.g., for treating a disease or disorder described herein, such as a prostate cancer described herein.
[16] The pharmaceutical composition of any one of [1]-[14], which has an oral bioavailability greater than 30% based on abiraterone plasma concentration profile, when tested in rats.
[17] A pharmaceutical composition comprising abiraterone decanoate dissolved in a lipid-based drug delivery system at a concentration ranging from about 10 mg/g to about 150 mg/g, wherein the lipid-based drug delivery system comprises (a) a lipid in an amount of about 10-80% by weight of the lipid-based drug delivery system; and (b) one or more non-ionic surfactants in an amount of about 20-90% by weight of the lipid-based drug delivery system, wherein abiraterone decanoate has the following structure:

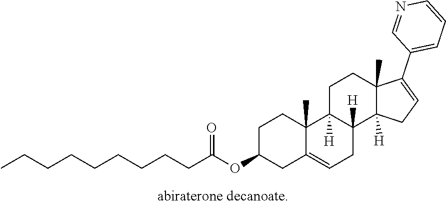

[18] The pharmaceutical composition of [17], wherein the lipid comprises medium-chain triglycerides of caprylic (C8) and capric (C10) acids (e.g., Labrafac™ lipophile WL 1349), for example, in an amount of about 10% to about 50% by weight of the lipid-based drug delivery system, such as about 20-40% by weight.
[19] The pharmaceutical composition of [17] or [18], wherein the lipid comprises glycerol/glyceryl linoleate (e.g., Maisine® CC), for example, in an amount of about 10% to about 50% by weight of the lipid-based drug delivery system, such as about 20-40% by weight.
[20] The pharmaceutical composition of any of [17]-[19], wherein the lipid further comprises propylene glycol monocaprylate (e.g., Capmul PG-8) or propylene glycol monolaurate (e.g., Capmul PG-12, or Lauroglycol™ 90 for example, in an amount of about 10% to about 50% by weight of the lipid-based drug delivery system, such as about 20-40% by weight.
[21] The pharmaceutical composition of any of [17]-[20], wherein the lipid-based drug delivery system comprises two or more, such as two or three, non-ionic surfactants.
[22] The pharmaceutical composition of any of [17]-[21], wherein the one or more non-ionic surfactants comprise macrogolglycerol hydroxystearate (e.g., Kolliphor RH 40) and/or polyglyceryl oleate (e.g., Plurol Oleique CC 497 (polyglyceryl-3 dioleate)).
[23] The pharmaceutical composition of any of [17]-[22], wherein the one or more non-ionic surfactants further comprise oleoyl polyoxyl-6 glycerides (e.g., Labrafil® M 1944 CS) and/or lauroyl polyoxyl-6 glycerides (e.g., Labrafil 2130).
[24] The pharmaceutical composition of any of [17]-[23], which comprises abiraterone decanoate dissolved in the lipid-based drug delivery system at a concentration ranging from about 20 mg/g to about 120 mg/g, wherein the lipid-based drug delivery system comprises (a) medium-chain triglycerides of caprylic (C8) and capric (C10) acids in an amount of about 20-40% by weight of the lipid-based drug delivery system; (b) macrogolglycerol hydroxystearate (e.g., Kolliphor RH 40) in an amount of about 10-30% by weight of the lipid-based drug delivery system; (c) polyglyceryl oleate (e.g., Plurol Oleique CC 497 (polyglyceryl-3 dioleate)) in an amount of about 10-30% by weight of the lipid-based drug delivery system; and (d) oleoyl polyoxyl-6 glycerides (e.g., Labrafil® M 1944 CS) in an amount of about 20-40% by weight of the lipid-based drug delivery system.
[25] The pharmaceutical composition of any of [17]-[23], which comprises abiraterone decanoate dissolved in the lipid-based drug delivery system at a concentration ranging from about 20 mg/g to about 120 mg/g, wherein the lipid-based drug delivery system comprises (a) medium-chain triglycerides of caprylic (C8) and capric (C10) acids in an amount of about 10-40% by weight of the lipid-based drug delivery system; (b) macrogolglycerol hydroxystearate (e.g., Kolliphor RH 40) in an amount of about 10-30% by weight of the lipid-based drug delivery system; (c) polyglyceryl oleate (e.g., Plurol Oleique CC 497 (polyglyceryl-3 dioleate)) in an amount of about 10-30% by weight of the lipid-based drug delivery system; (d) oleoyl polyoxyl-6 glycerides (e.g., Labrafil® M 1944 CS) in an amount of about 10-40% by weight of the lipid-based drug delivery system; and (e) propylene glycol monocaprylate (e.g., Capmul PG-8) and/or propylene glycol monolaurate (e.g., Capmul PG-12, or Lauroglycol™ 90) in an amount of about 10-40% by weight of the lipid-based drug delivery system.
[26] The pharmaceutical composition of any of [17]-[23], which comprises abiraterone decanoate dissolved in the lipid-based drug delivery system at a concentration ranging from about 20 mg/g to about 120 mg/g, wherein the lipid-based drug delivery system comprises (a) medium-chain triglycerides of caprylic (C8) and capric (C10) acids in an amount of about 10-40% by weight of the lipid-based drug delivery system; (b) macrogolglycerol hydroxystearate (e.g., Kolliphor RH 40) in an amount of about 10-30% by weight of the lipid-based drug delivery system; (c) polyglyceryl oleate (e.g., Plurol Oleique CC 497 (polyglyceryl-3 dioleate)) in an amount of about 10-30% by weight of the lipid-based drug delivery system; (d) oleoyl polyoxyl-6 glycerides (e.g., Labrafil® M 1944 CS) in an amount of about 0-40% by weight of the lipid-based drug delivery system; and (e) glycerol/glyceryl linoleate (e.g., Maisine® CC, mono-, di- and triglycerides of mainly linoleic (C_{18:2}) and oleic (C_{18:1}) acids, the diester fraction being predominant) in an amount of about 10-40% by weight of the lipid-based drug delivery system.
[27] The pharmaceutical composition of [17], which comprises a vehicle described in any of the examples herein.
[28] The pharmaceutical composition of any one of [17]-[27], formulated for oral administration, such as in the form of a capsule (e.g., a soft gel capsule).
[29] The pharmaceutical composition of any one of [17]-[28], which is characterized by one or more of the following: (1) the pharmaceutical composition is storage stable at room temperature; (2) the recovery of abiraterone decanoate is greater than 50% when the pharmaceutical composition is assessed using an in vitro dispersion test; and (3) upon oral administration to a mammal, the pharmaceutical composition is capable of delivering a sufficient amount of abiraterone decanoate to the mammal to achieve a therapeutically effective plasma concentration of abiraterone, e.g., for treating a disease or disorder described herein, such as a prostate cancer described herein.
[30] The pharmaceutical composition of any one of [17]-[29], which has an oral bioavailability of greater than 30% based on abiraterone plasma concentration profile, when tested in rats.
[31] The pharmaceutical composition of any one of [1]-[30], wherein the lipid-based drug delivery system is a self-dispersing drug delivery system, such as a self-emulsifying drug delivery system or self-microemulsifying drug delivery system.
[32] The pharmaceutical composition of any one of [1]-[31], wherein upon oral administration to a mammal, at least a portion of the abiraterone decanoate is absorbed through the lymphatic system.
[33] The pharmaceutical composition of any one of [1]-[32], wherein the abiraterone decanoate is substantially pure, e.g., characterized as having a purity by weight of at least 95%, preferably, at least 98%, such as about 98.5%, about 99%, about 99.5%, or higher.
[34] The pharmaceutical composition of [33], wherein the abiraterone decanoate is characterized as having less than 1% (e.g., less than 0.5% by weight, such as less than 0.3%, less than 0.2%, or less than 0.1%) by weight of ethyl prasterone decanoate having the formula:

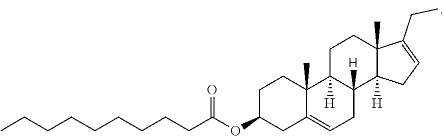

[35] The pharmaceutical composition of [33], wherein the abiraterone decanoate is characterized as having no detectable amount of ethyl prasterone decanoate.
[36] The pharmaceutical composition of any one of [33]-[35], wherein the abiraterone decanoate is characterized as having a Palladium content of less than 50 ppm.
[37] The pharmaceutical composition of any one of [33]-[35], wherein the abiraterone decanoate is characterized as having a Palladium content of less than 10 ppm.
[38] A method of treating or preventing a disease or disorder in a subject in need thereof, the method comprising administering to the subject an effective amount of the pharmaceutical composition according to any one of [1]-[37], wherein the disease or disorder is selected from a sex hormone-dependent benign or malignant disorder, an androgen receptor driven cancer, a syndrome due to androgen excess, and a syndrome due to glucocorticoid excess.
[39] The method of [38], wherein the disease or disorder is selected from prostate cancer, breast cancer, endometrial cancer, ovarian cancer, bladder cancer, hepatocellular carcinoma, lung cancer, endometriosis, polycystic ovary syndrome, Cushing's syndrome, Cushing's disease, classical or nonclassical congenital adrenal hyperplasia, precocious puberty, hirsutism, and combinations thereof.
[40] The method of [38], wherein the disease or disorder is a sex hormone dependent or androgen receptor driven cancer.
[41] The method of [40], wherein the sex hormone dependent or androgen receptor driven cancer is androgen receptor positive salivary duct carcinoma, or androgen receptor positive glioblastoma multiforme.
[42] The method of [38], wherein the disease or disorder is prostate cancer.
[43] The method of [42], wherein the subject having prostate cancer is characterized as having a rising amount of prostate specific antigen, e.g., following radical prostatectomy.
[44] The method of [42], wherein the prostate cancer is a localized prostate cancer, e.g., a high risk localized prostate cancer.
[45] The method of [42], wherein the prostate cancer is a metastatic castration-sensitive prostate cancer, non-metastatic castration-sensitive prostate cancer, non-metastatic castration-resistant prostate cancer, or metastatic castration-resistant prostate cancer.
[46] The method of [42], wherein the prostate cancer is a newly diagnosed high risk metastatic hormone sensitive prostate cancer.
[47] The method of [42], wherein the prostate cancer is a metastatic castration resistant prostate cancer (mCRPC), wherein the subject is asymptomatic or mildly symptomatic after failure of androgen deprivation therapy in whom chemotherapy is not yet clinically indicated.
[48] The method of [42], wherein the prostate cancer is a metastatic castration resistant prostate cancer (mCRPC), wherein the subject's disease has progressed on or after a taxane-based such as docetaxel-based chemotherapy regimen.
[49] The method of [42], wherein the prostate cancer is a refractory prostate cancer.
[50] The method of any one of [38]-[49], further comprising treating the subject with radiotherapy or surgery.
[51] The method of any one of [38]-[50], further comprising administering to the subject one or more other agents selected from anticancer agents, hormone ablation agents, anti-androgen agents, differentiating agents, anti-neoplastic agents, kinase inhibitors, anti-metabolite agents, alkylating agents, antibiotic agents, immunological agents, interferon-type agents, intercalating agents, growth factor inhibitors, cell cycle inhibitors, enzymes, topoisomerase inhibitors, biological response modifiers, mitotic inhibitors, matrix metalloprotease inhibitors, genetic therapeutics, or combinations thereof.
[52] The method of any one of [38]-[51], further comprising administering to the subject one or more agents selected from hydrocortisone, prednisone, prednisolone, methylprednisolone, and dexamethasone.
[53] The method of any one of [38]-[52], further comprising administering to the subject one or more other agents selected from a chemotherapeutic drug, hormone replacement drug, or hormone ablation drug.
[54] The method of any one of [38]-[53], further comprising treating the subject with an androgen deprivation therapy.
[55] The method of any one of [38]-[53], wherein the subject is a non-castrated subject.
[56] The method of any one of [38]-[53], wherein the subject is not treated with a gonadotropin-releasing hormone agonist and/or antagonist in an amount effective to reduce serum testosterone level in the subject.
[57] The method of [56], wherein the subject is not treated with a drug selected from buserelin, leuprolide, deslorelin, fertirelin, histrelin, gonadorelin, lecirelin, goserelin, nafarelin, peforelin and triptorelin.
[58] The method of [56], wherein the subject is not treated with a drug selected from abarelix, cetrorelix, degarelix, ganirelix, elagolix, linzagolixa, and relugolix.
[59] The method of any one of [55]-[58], wherein the subject is sensitive to or otherwise intolerant with a gonadotropin-releasing hormone antagonist and/or agonist.
[60] The method of any one of [55]-[59], wherein the subject is not treated with a glucocorticoid replacement therapy.
[61] The method of any one of [38]-[60], further comprising administering to the subject a poly ADP ribose polymerase (PARP) inhibitor, e.g., niraparib, rucaparib, olaparib, talazoparib, veliparib, and fluzoparib.
[62] The method of any one of [38]-[61], further comprising administering to the subject a 1^{st}-generation androgen receptor antagonist, e.g., proxalutamide, bicalutamide, flutamide, nilutamide, topilutamide.
[63] The method of any one of [38]-[62], further comprising administering to the subject a 2^{nd}-generation androgen receptor antagonist (e.g., apalutamide, darolutamide or enzalutamide).
[64] The method of any one of [38]-[63], further comprising administering to the subject a 3^{rd} generation androgen receptor antagonist (such as an N-terminal domain inhibitor) or an androgen receptor degrader molecule, alone or in combination with one or more 1^{st} generation or 2^{nd} generation androgen receptor antagonists.
[65] The method of any one of [38]-[64], further comprising administering to the subject a chemotherapeutic agent, such as a taxane based chemotherapeutic agent (e.g., docetaxel, cabazitaxel, paclitaxel, etc.) or platinum based chemotherapeutic agent (e.g., cisplatin, carboplatin, oxaliplatin, etc.).
[66] The method of any one of [38]-[65], further comprising administering to the subject an immunotherapy, such as administering Sipuleucel-T, an immune checkpoint inhibitor (e.g., anti-PD-1 antibody such as pembrolizumab or nivolumab, or anti-PD-L1 antibody such as avelumab or atezolizumab), or an anti-CTLA-4 antibody (e.g., ipilimumab), etc.
[67] The method of any one of [38]-[66], further comprising administering to the subject a bispecific T-cell engager (BiTE) therapy, such as blinatumomab or solitomab.
[68] The method of any one of [38]-[67], further comprising administering to the subject a kinase inhibitor, e.g., sunitinib, dasatinib, cabozantinib, erdafitinib, dovitinib, capivasertib, onvansertib, ipatasertib, afuresertib, alisertib, apitolisib, opaganib, etc.
[69] The method of any one of [38]-[68], further comprising administering to the subject a bone protecting agent (e.g., denosumab, zolendronic acid), and wherein the subject is characterized as having prostate cancer (e.g., CRPC) with bone metastasis.
[70] The method of any one of [38]-[69], further comprising administering to the subject a therapeutic agent selected from 1) an anti-IL23 targeting monoclonal antibody, e.g., tildrakizumab; 2) a selenium, such as sodium selenite; 3) an EZH2 inhibitor, e.g., CPI-1205, GSK2816126, or tazemetostat; 4) a CDK4/6 inhibitor, e.g., palbociclib, ribociclib, abemaciclib; 6) a bromodomain and extra-terminal domain (BET) inhibitor, e.g., CCS1477, INCB057643, alobresib, ZEN-3694, or molibresib (GSK525762); 7) an anti-CD105 antibody, e.g., TRC105 or carotuximab; 8) niclosamide; 9) an A2A receptor antagonist, e.g., AZD4635; 10) a PI3K inhibitor, e.g., AZD-8186, buparlisib, or dactolisib; 11) a further non-steroidal CYP17A1 inhibitor, e.g. seviteronel; 12) an antiprogestogen, e.g., onapristone; 13) navitoclax; 14) an HSP90 inhibitor, e.g., onalespib (AT13387); 15) an HSP27 inhibitor, e.g., OGX-427; 16) a 5-alpha-reductase inhibitor, e.g., dutasteride; 17) metformin; 18) AMG-386; 19) dextromethorphan; 20) theophylline; 21) hydroxychloroquine; and 22) lenalidomide.
[71] The method of any one of [38]-[70], further comprising administering to the subject one or more kinase modulators selected from FLT-3 (FMS-like tyrosine kinase) inhibitors, AXL (anexelekto) inhibitors (e.g., Gilteritinib), CDK (cyclin dependent kinase) inhibitors, such as CDK1, 2, 4, 5, 6, 7, or 9 inhibitors, retinoblastoma (Rb) inhibitors, protein kinase B (AKT) inhibitors, SRC inhibitors, IkappaB kinase 1 (IKK1) inhibitors, PIM-1 modulators, Lemur tyrosine kinase 2 (LMTK2) modulators, Lyn inhibitors, Aurora A inhibitors, ANPK (a nuclear protein kinase) inhibitors, extracellular-signal regulated kinase (ERK) modulators, c-jun N-terminal kinase (JNK) modulators, Big MAP kinase (BMK) modulators, p38 mitogen-activated protein kinases (MAPK) modulators, and combinations thereof.
[72] The method of any one of [38]-[71], wherein the subject is chemotherapy naïve or hormone therapy naïve prior to being administered the pharmaceutical composition.
[73] The method of any one of [38]-[72], wherein the subject has not undergone a prostatectomy.
[74] The method of any one of [38]-[73], wherein the subject is treated with radiotherapy e.g., stereotactic body radiotherapy, neutron radiation.
[75] The method of any one of [38]-[74], wherein the subject is administered Radium-223.
[76] The method of [38], wherein the disease or disorder is breast cancer, e.g., molecular apocrine HER2-negative breast cancer, metastatic breast cancer, such as ER+ metastatic breast cancer, ER+ and HER2 negative breast cancer, AR+ triple negative breast cancer, etc.
[77] The method of [76], further comprising administering to the subject an aromatase inhibitor, e.g., exemestane.
[78] The method of [38], wherein the disease or disorder is associated with 21-hydroxylase deficiency.
[79] The method of any one of [38]-[78], wherein the pharmaceutical composition is administered orally.
[80] The method of any one of [38]-[79], wherein the pharmaceutical composition is administered to the subject ranging from once a day to once a week, such as once a day or once every two or three days.
[81] The method of any one of [38]-[80], wherein the pharmaceutical composition is administered to the subject with or without food.
[82] An emulsion comprising (a) abiraterone decanoate; (b) a lipid; and (c) a non-ionic surfactant, wherein the lipid phase of the emulsion comprises abiraterone decanoate dispersed in the lipid, wherein abiraterone decanoate has the following structure:

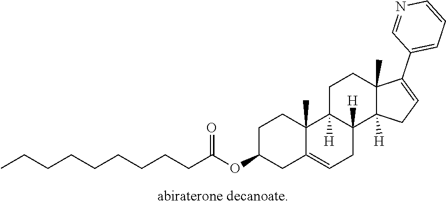

[83] The emulsion of [82], wherein the lipid comprises medium-chain triglycerides of caprylic (CS) and capric (C10) acids (e.g., Labrafac™ lipophile WL 1349).
[84] The emulsion of [82] or [83], wherein the lipid comprises glycerol/glyceryl linoleate (e.g., Maisine® CC).
[85] The emulsion of any of [82]-[84], wherein the lipid further comprises propylene glycol monocaprylate (e.g., Capmul PG-8) or propylene glycol monolaurate (e.g., Capmul PG-12, or Lauroglycol™ 90).
[86] The emulsion of any of [82]-[85], comprising two or more, such as two or three, non-ionic surfactants.
[87] The emulsion of any of [82]-[86], wherein the non-ionic surfactant comprises macrogolglycerol hydroxystearate (e.g., Kolliphor RH 40) and/or polyglyceryl oleate (e.g., Plurol Oleique CC 497 (polyglyceryl-3 dioleate)).
[88] The emulsion of [87], wherein the surfactant further comprises oleoyl polyoxyl-6 glycerides (e.g., Labrafil® M 1944 CS) and/or lauroyl polyoxyl-6 glycerides (e.g., Labrafil 2130).
[89] An emulsion produced by mixing the pharmaceutical composition of any one of [1]-[37] with water.
[90] An emulsion produced by administering the pharmaceutical composition of any one of [1]-[37] to a mammal.
[91] A method of treating or preventing a disease or disorder in a subject in need thereof, the method comprising administering to the subject an effective amount of the emulsion according to any one of [82]-[90], wherein the disease or disorder is selected from a sex hormone-dependent benign or malignant disorder, an androgen receptor driven cancer, a syndrome due to androgen excess, and a syndrome due to glucocorticoid excess.

Embodiments of the present disclosure can fulfill a long felt need in the field of sex hormone-dependent disorders and oncology including the treatment of a sex hormone dependent or androgen receptor driven cancer such as prostate cancer. Embodiments of the present disclosure can also fulfill a long felt need in the field of treating syndromes due to androgen excess syndrome and/or due to glucocorticoid excess such as hypercortisolemia. Embodiments of the present disclosure can overcome major disadvantages and deficiencies of prior art formulations (including commercially-available oral dosage forms) of abiraterone acetate, by providing novel oral formulations of abiraterone prodrugs, methods of producing the same, methods of treatment using the same, and kits for convenient administration of the formulations to subjects in need of therapy for various disorders including prostate cancer.

There has thus been outlined, rather broadly, features in order that the detailed description thereof that follows may be better understood, and in order that the present contribution to the art may be better appreciated. There are, of course, additional features that will be described further hereinafter. Indeed, it is to be understood that both the foregoing general description and the following detailed description are exemplary and explanatory and are intended to provide further explanation of the disclosure.

In this respect, before explaining at least one embodiment in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those persons skilled in the art will appreciate that the conception upon which this disclosure is based can readily be utilized as a basis for the designing of other formulations, methods, systems, kits, and compositions for carrying out the several purposes of the present disclosure. It is important, therefore, that equivalent constructions insofar as they do not depart from the spirit and scope of the present disclosure, are included in the present disclosure.

The accompanying drawings are included to provide a further understanding and are incorporated in and constitute a part of this specification, illustrate several embodiments, and together with the description explain the principles.

### BRIEF DESCRIPTION OF THE DRAWINGS

FIG. 1 presents biochemical pathways showing the effects of CYP17A1 inhibition on the synthesis of androgens, estrogens, glucocorticoids, progesterone, and mineralocorticoids.

FIG. 2A presents a representative X-ray Powder Diffraction (XRPD) spectrum of the abiraterone decanoate (alternatively abbreviated herein as “AbiDec”, “ADEC”, or “AbDec”) solid form prepared in Example 1A, designated as Form A.

FIG. 2B shows a representative Differential Scanning Calorimetry (DSC) spectrum of the abiraterone decanoate solid form prepared in Example 1A, designated as Form A.

FIG. 2C shows a representative thermogravimetric analysis (TGA) of the abiraterone decanoate solid form prepared in Example 1A, designated as Form A.

FIG. 2D presents a representative XRPD spectrum of the abiraterone decanoate in Form B.

FIG. 2E shows a representative DSC spectrum of the abiraterone decanoate in Form B.

FIG. 2F shows a representative TGA of the abiraterone decanoate in Form B.

FIG. 2G presents a representative XRPD spectrum of the abiraterone decanoate in Form C.

FIG. 2H shows a representative DSC spectrum of the abiraterone decanoate in Form C.

FIG. 2I shows a representative TGA of the abiraterone decanoate in Form C.

FIG. 3 presents a graph showing the mean plasma concentrations of abiraterone and abiraterone decanoate in plasma following oral administration of abiraterone decanoate in 2 different formulations. The ranking from the highest Cₘₐₓ to lowest Cₘₐₓ in absolute values is the following: abiraterone from Group 2 treatment, abiraterone from Group 3 treatment, abiraterone decanoate from Group 2 treatment, and abiraterone decanoate from Group 3 treatment.

FIG. 4A presents a graph showing the mean plasma concentrations of progesterone (“Prog”) following oral administration of vehicle (Group 1 or Grp 1), formulation 1 (Group 2 or Grp 2) and formulation 2 (Group 3 or Grp 3).

FIG. 4B presents a graph showing the mean plasma concentrations of progesterone (“Prog”) following oral administration of formulation 1 (Group 2 or Grp 2) and formulation 2 (Group 3 or Grp 3) relative to that from oral administration of vehicle (Group 1 or Grp 1).

FIG. 5A presents a graph showing the mean plasma concentrations of corticosterone (“Cortico”) following oral administration of vehicle (Group 1 or Grp 1), formulation 1 (Group 2 or Grp 2) and formulation 2 (Group 3 or Grp 3).

FIG. 5B presents a graph showing the mean plasma concentrations of corticosterone (“Cortico”) following oral administration of formulation 1 (Group 2 or Grp 2) and formulation 2 (Group 3 or Grp 3) relative to that from oral administration of vehicle (Group 1 or Grp 1).

FIG. 6A presents a graph showing the mean plasma concentrations of androstenedione (“Andro”) following oral administration of vehicle (Group 1 or Grp 1), formulation 1 (Group 2 or Grp 2) and formulation 2 (Group 3 or Grp 3).

FIG. 6B presents a graph showing the mean plasma concentrations of androstenedione (“Andro”) following oral administration of formulation 1 (Group 2 or Grp 2) and formulation 2 (Group 3 or Grp 3) relative to that from oral administration of vehicle (Group 1 or Grp 1).

FIG. 7A presents a graph showing the mean plasma concentrations of testosterone (“T”) following oral administration of vehicle (Group 1 or Grp 1), formulation 1 (Group 2 or Grp 2) and formulation 2 (Group 3 or Grp 3).

FIG. 7B presents a graph showing the mean plasma concentrations of testosterone (“T”) following oral administration of formulation 1 (Group 2 or Grp 2) and formulation 2 (Group 3 or Grp 3) relative to that from oral administration of vehicle (Group 1 or Grp 1).

FIG. 8 presents a graph showing the mean plasma concentrations of luteinizing hormone following oral administration of vehicle (Group 1 or Grp 1), formulation 1 (Group 2 or Grp 2) and formulation 2 (Group 3 or Grp 3). The ranking based on the last observed concentration (at 72 hours) is the following: Group 2>Group 3>Group 1.

FIG. 9 presents a bar graph showing the mean tissue concentrations of abiraterone in tissues following oral administration of formulation 1 (Group 2 or Grp 2) and formulation 2 (Group 3 or Grp 3).

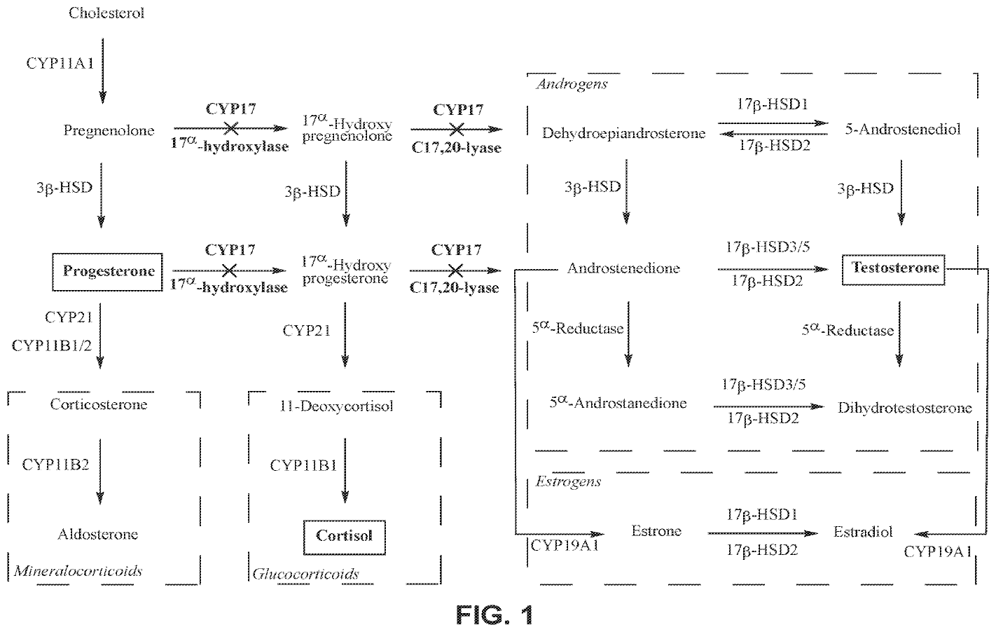

### DETAILED DESCRIPTION

In a broad aspect, the present disclosure relates to novel oral abiraterone prodrug formulations, methods for modulating serum steroid hormone levels in a subject in need thereof and methods for treating or preventing a disease or disorder associated with such steroid hormones. Embodiments of the present disclosure are based, in part, on that abiraterone prodrugs, in particular, abiraterone decanoate, can be administered to a subject orally to achieve sustained inhibition of CYP17A1 activities and reduction of serum androgen levels.

As detailed in U.S. Pat. No. 10,792,292 B2, and U.S. Provisional Application Nos. 63/073,502 and 63/149,550, the contents of each of which is herein incorporated by reference in its entirety, parenteral administration of abiraterone prodrugs such as abiraterone decanoate can be advantageous over existing methods in many aspects, including but not limited to a fast and sustained reduction of serum testosterone, no need for castration, reduced or no liver toxicity compared to methods using oral abiraterone acetate formulations, improved bioavailability, elimination of the food effect associated with oral abiraterone acetate formulation, reduced pill burden, better patient compliance, decreased dosing frequency, sustained stable blood levels of active drug, reduced Cₘₐₓ, which can reduce associated side effects, etc.

As discussed herein, it is found that with proper lipid-based drug delivery system, abiraterone decanoate can be orally bioavailable with good to excellent bioavailability to achieve an effective plasma concentration of abiraterone for modulating serum steroid level, such as reduction of serum androgen levels. This provides alternative methods for administering abiraterone prodrugs, such as abiraterone lipophilic esters, in particular, abiraterone decanoate, to treat various diseases or disorders described herein, such as a prostate cancer described herein.

Accordingly, in various embodiments, the present disclosure provides novel oral abiraterone prodrug formulations, methods for modulating serum steroid hormone levels, such as for reducing testosterone levels, and/or novel methods for treating or preventing diseases or disorders mediated by or associated with such steroids, such as sex hormone dependent or androgen receptor driven cancers.

### Abiraterone Decanoate Formulations

In some embodiments, the present disclosure provides various formulations comprising abiraterone prodrugs, such as abiraterone lipophilic esters, in particular, abiraterone decanoate, which has the following structure:

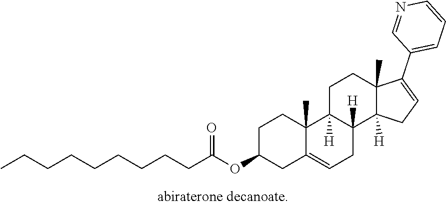

Typically, the pharmaceutical composition herein comprises (a) abiraterone decanoate; and (b) a lipid-based drug delivery system, wherein the pharmaceutical composition is formulated for oral delivery of abiraterone decanoate.

The lipid-based drug delivery system herein broadly refers to any lipid-based vehicle, which when formulated with abiraterone decanoate, is suitable for oral administration to deliver a sufficient amount of abiraterone decanoate to achieve an effective plasma concentration of abiraterone for inhibiting CYP17A1, for modulation of various steroid hormone levels, such as androgens, estrogens, glucocorticoids, progesterone, and mineralocorticoids, and/or for treating a disease or disorder described herein, such as a prostate cancer described herein. The lipid-based drug delivery system herein can typically be characterized as a self-dispersing drug delivery system, such as a self-emulsifying drug delivery system or self-microemulsifying drug delivery system. Typically, the abiraterone decanoate is homogeneous dispersed or dissolved in the lipid-based drug delivery system.

In some embodiments, the pharmaceutical composition herein is characterized as being capable of delivering at least a portion of the abiraterone decanoate through the lymphatic delivery system upon oral administration to a mammal. Lymphatic delivery of lipid-based drug delivery system can bypass the first-pass metabolism and therefore can lead to better overall pharmacokinetic profile. See general discussions of lymphatic delivery in, Punjabi et al. Current Pharmaceutical Design, 27:1992-1998 (2021); Bora et al. Indian Drugs 54(08):5-22 (2017); Pouton et al. Advanced Drug Delivery Reviews 60:625-637 (2008); and Cote et al. Advanced Drug Delivery Reviews 144:16-34 (2019). As shown in the Examples section, a single administration of exemplary oral formulations herein achieved excellent oral bioavailability in rat pharmacokinetic studies, which also potently reduced circulating androgen levels (androstenedione and testosterone) and increased the levels of progesterone. Without wishing to be bound by theories, it is believed that upon oral administration, the pharmaceutical composition comprising abiraterone decanoate herein (e.g., any of those described herein, such as [1]-[37] of the Summary section herein) to a mammal, at least a portion of the abiraterone decanoate is absorbed through the lymphatic system. The lymphatic delivery is also supported by the tissue study herein which showed that abiraterone was detected in tissues at 72 h post dose, in mandibular and mesenteric lymph nodes.

The pharmaceutical composition herein is typically formulated in the form of an oral dosage form, such as a capsule (e.g., a soft gel capsule).

The pharmaceutical composition herein (e.g., any of those described herein, such as [1]-[37] of the Summary section herein) is typically also characterized by one or more of the following: (1) the pharmaceutical composition is storage stable at room temperature; (2) the recovery of abiraterone decanoate is greater than 50% when the pharmaceutical composition is assessed using an in vitro dispersion test; and (3) upon oral administration to a mammal, the pharmaceutical composition is capable of delivering a sufficient amount of abiraterone decanoate to the mammal to achieve a therapeutically effective plasma concentration of abiraterone, e.g., for treating a disease or disorder described herein, such as a prostate cancer described herein. As used herein, the “in vitro dispersion test” should be understood as using a procedure in accordance with the procedures described in Example 4 of this application. By “storage stable”, it is meant that the pharmaceutical composition, upon storage at a storage condition for a storage period, for example, at room temperature for 1 month or longer (e.g., about 1 month, about 3 months, about 6 months, or longer), has (1) substantially the same amount of abiraterone decanoate, e.g., within 80-125% of the amount at the start of the storage; (2) substantially the same amount of impurities (total and/or individual impurity), e.g., within 80-125% of the amount at the start of the storage; and/or (3) no substantial physical property changes, for example, the appearance and dispersibility in aqueous solution remain substantially the same as the start of the storage.

In preferred embodiments, the pharmaceutical composition herein (e.g., any of those described herein, such as [1]-[37] of the Summary section herein) can have an oral bioavailability greater than 30% based on abiraterone plasma concentration profile, when tested in rats. Oral bioavailability can be readily determined by those skilled in the art. Exemplary methods for determining rat oral bioavailability are shown in the Examples section herein.

### Lipid-Based Drug Delivery System

The lipid-based drug delivery system herein typically includes one or more lipids and one or more surfactants. Suitable lipids and surfactants and their amounts include those that are generally accepted for pharmaceutical uses, such as those described in the inactive ingredient database from the U.S. Food and Drug Administration. Particular lipids and surfactants for the pharmaceutical compositions herein can be typically selected based on the solubility of abiraterone decanoate, stability of the pharmaceutical composition, excipient compatibility, and dispersibility of the pharmaceutical composition in aqueous solution, etc. For example, in some embodiments, the lipids and surfactants are selected such that the pharmaceutical composition can have abiraterone decanoate dissolved or suspended in the lipid-based drug delivery system at a concentration of about 1 mg/g to about 250 mg/g. To be clear, a concentration of 250 mg/g means that for each gram of a mixture of abiraterone decanoate in the lipid-based drug delivery system, there is 250 mg of abiraterone decanoate. Concentrations of abiraterone decanoate in the lipid-based drug delivery system described elsewhere in this disclosure should be understood similarly. Preferably, the lipids and surfactants are selected such that the pharmaceutical composition can have abiraterone decanoate dissolved in the lipid-based drug delivery system at a concentration of about 20 mg/g to about 150 mg/g. In some embodiments, the lipids and surfactants are selected such that the pharmaceutical composition is storage stable at room temperature, for example, for 1 month, 3 months, 6 months, or longer. In some embodiments, the lipids and surfactants are selected such that the pharmaceutical composition comprises a solution (or otherwise a homogenous mixture) of abiraterone decanoate in the lipid-based drug delivery system at room temperature, wherein the solution (or otherwise homogenous mixture) can remain a solution (or otherwise homogenous), i.e., no visible formation of drug and/or excipient crystals/precipitations, after storage at room temperature for 1 month, 3 months, 6 months, or longer. In some embodiments, the lipids and surfactants are selected such that the recovery of abiraterone decanoate is greater than 50% (e.g., 55%, 60%, 70%, 80%, 90%, or up to 100%, or any range between the recited values) when the pharmaceutical composition is assessed using an in vitro dispersion test. In some embodiments, the lipids and surfactants are selected such that upon oral administration to a mammal, the pharmaceutical composition is capable of delivering a sufficient amount of abiraterone decanoate to the mammal to achieve a therapeutically effective plasma concentration of abiraterone, e.g., for treating a disease or disorder described herein, such as a prostate cancer described herein. In some embodiments, the lipids and surfactants are selected such that upon oral administration to a mammal, the pharmaceutical composition is capable of delivering a sufficient amount of abiraterone decanoate to the mammal to achieve an effective plasma concentration of abiraterone, e.g., for inhibiting CYP17A1. In some embodiments, the lipids and surfactants are selected such that the pharmaceutical composition herein can have an oral bioavailability greater than 30%, e.g., up to 60%, 70% or higher, based on abiraterone plasma concentration profile, when tested in rats.

Typically, the lipid for the lipid-based drug delivery system comprises a triglyceride, monoglyceride, diglyceride, and/or a propylene glycol ester. The surfactant for the lipid-based drug delivery system typically comprises one or more non-ionic surfactants.

In some embodiments, the lipid-based drug delivery system herein comprises (1) a lipid comprising a triglyceride, monoglyceride, diglyceride, and/or a propylene glycol ester; and (2) a surfactant, such as a non-ionic surfactant. The surfactant can be any of those described herein, for example, the surfactant can comprise a polyglyceryl ester and/or polyoxyglyceride. Triglycerides, monoglycerides, and diglycerides as used herein should be understood as mono-, di-, or tri-esters of glycerol of fatty acids, and propylene glycol esters as used herein should be understood as mono or di esters of propylene glycol of fatty acids, wherein the fatty acids can typically be, but not limited to, medium or long-chained fatty acids, which can for example be saturated or unsaturated. Those skilled in the art would understand that medium-chained fatty acids include an aliphatic tail of 6-12 carbons, and long-chained fatty acids include an aliphatic tail of 13-21 carbons. Some of the triglycerides, monoglycerides, diglycerides, or propylene glycol esters may also be viewed as a surfactant. However, as used herein and to be clear, the surfactant for the lipid-based drug delivery system herein should be understood as requiring one or more surfactants that are not the triglycerides, monoglycerides, diglycerides, or propylene glycol esters defined herein. When calculating the weight percentages of the surfactants in the lipid-based drug delivery system or pharmaceutical composition herein, only those surfactants that are not the triglycerides, monoglycerides, diglycerides, or propylene glycol esters should be considered, unless otherwise specified or contrary from context.

In some embodiments, the lipid-based drug delivery system herein comprises (1) a triglyceride, monoglyceride, diglyceride, and/or a propylene glycol ester; and (2) a surfactant comprising a polyglyceryl ester and/or polyoxyglyceride. In some embodiments, the lipid-based drug delivery system herein comprises a triglyceride and the surfactant. In some embodiments, the lipid-based drug delivery system herein comprises a diglyceride and the surfactant. In some embodiments, the lipid-based drug delivery system herein comprises a monoglyceride and the surfactant. In some embodiments, the lipid-based drug delivery system herein comprises a propylene glycol ester and the surfactant. In some embodiments, the lipid-based drug delivery system herein comprises (1) a triglyceride and one or more selected from monoglyceride, diglyceride, and propylene glycol ester and (2) the surfactant. In some embodiments, the lipid-based drug delivery system herein comprises (1) a triglyceride and propylene glycol ester and (2) the surfactant. In some embodiments, the lipid-based drug delivery system herein comprises (1) a triglyceride, monoglyceride, and diglyceride; and (2) the surfactant. In some embodiments, the lipid-based drug delivery system herein comprises (1) a triglyceride, monoglyceride, diglyceride, and propylene glycol ester and (2) the surfactant. Suitable triglycerides, monoglycerides, diglycerides, propylene glycol esters, and surfactants include any of those described herein in any combinations.

In some embodiments, the lipid-based drug delivery system herein comprises (1) a medium-chain triglyceride; and (2) a surfactant, such as a non-ionic surfactant. The medium-chain triglyceride is not particularly limited. For example, in some embodiments, the medium-chain triglyceride can be medium-chain triglycerides of caprylic (C8) and capric (C10) acids, such as those commercially available under the tradename: Labrafac™ lipophile WL 1349. In some embodiments, the surfactant comprises a polyglyceryl ester and/or polyoxyglyceride.

In some embodiments, the lipid-based drug delivery system herein comprises a monoglyceride and/or diglyceride. For example, in some embodiments, the lipid-based drug delivery system comprises a glycerol/glyceryl linoleate, such as those from a commercial available product under the tradename: Maisine® CC, which is believed to have mono-, di- and triglycerides of mainly linoleic (C18:2) and oleic (C18:1) acids, with the diester fraction being predominant.

In some embodiments, the lipid-based drug delivery system herein comprises a propylene glycol ester. Suitable propylene glycol esters include for example propylene glycol monocaprylate (e.g., sold under the tradename: Capmul PG-8) and/or propylene glycol monolaurate (e.g., sold under the tradename: Capmul PG-12, or Lauroglycol™ 90).

In some embodiments, the lipid-based drug delivery system herein comprises medium-chain triglycerides of caprylic (C8) and capric (C10) acids (e.g., Labrafac™ lipophile WL 1349) and a surfactant as described herein.

In some embodiments, the lipid-based drug delivery system herein comprises (1) medium-chain triglycerides of caprylic (C8) and capric (C10) acids (e.g., Labrafac™ lipophile WL 1349); (2) glycerol/glyceryl linoleate (e.g., Maisine® CC, mono-, di- and triglycerides of mainly linoleic (C_{18:2}) and oleic (C_{18:1}) acids, the diester fraction being predominant); and a surfactant as described herein. In such embodiments (e.g., any of those applicable embodiments described herein, such as [6]-[16] and [19]-[37] of the Summary section herein), the weight ratio of medium-chain triglycerides to glycerol/glyceryl linoleate typically ranges from about 5:1 to 1:5, more typically ranges from about 2:1 to about 1:2, such as about 1.5:1, about 1:1, about 1:1.5, or about 1:2, or any range between the recited values.

In some embodiments, the lipid-based drug delivery system herein comprises (1) medium-chain triglycerides of caprylic (C8) and capric (C10) acids (e.g., Labrafac™ lipophile WL 1349); (2) propylene glycol monocaprylate (e.g., Capmul PG-8); and a surfactant as described herein. In such embodiments (e.g., any of those applicable embodiments described herein, such as [8]-[16] and [20]-[37] of the Summary section herein), the weight ratio of medium-chain triglycerides to propylene glycol monocaprylate typically ranges from about 5:1 to 1:5, more typically ranges from about 2:1 to about 1:2, such as about 1.5:1, about 1:1, about 1:1.5, or about 1:2, or any range between the recited values.

In some embodiments, the surfactant in the lipid-based drug delivery system can comprise a polyglycerol ester. Useful polyglycerol esters are not particularly limited, which include esters formed from fatty acids and homopolymers of glycerol, such as trimers, tetramers, etc. For example, in some embodiments, the surfactant in the lipid-based drug delivery system can comprise polyglyceryl oleate, such as polyglyceryl-3 dioleate, available commercially under the tradename: Plurol Oleique CC 497. Polyglyceryl-3 dioleate refers to the dioleate of a homotrimer of glycerine, with the main component having a molecular formula: C₄₅H₈₄O₉ or R—O—(CH₂—CH(OR)—CH₂—O)₃—R, wherein R=H, or CO—C₁₇H₃₃, CAS No.: 9007-48-1.

In some embodiments, the surfactant in the lipid-based drug delivery system can comprise a polyoxyglyceride. Useful polyoxyglyceride are not particularly limited, which include esters formed from fatty acids and ethoxylated glycerol. For example, in some embodiments, the surfactant in the lipid-based drug delivery system can comprise macrogolglycerol hydroxystearate, for example, those available commercially under the tradename: Kolliphor RH 40. In some embodiments, the surfactant in the lipid-based drug delivery system can comprise oleoyl polyoxyl-6 glycerides, for example, those available commercially under the tradename: Labrafil® M 1944 CS. Chemically, Labrafil® M 1944 CS can have mono-, di- and triglycerides and PEG-6 (MW 300) mono- and diesters of oleic (C_{18:1}) acid. In some embodiments, the surfactant in the lipid-based drug delivery system can comprise lauroyl polyoxyl-6 glycerides, for example, those available commercially under the tradename: Labrafil® 2130 CS. Chemically, Labrafil® 2130 CS can have mono-, di- and triglycerides and PEG-6 (MW 300) mono- and diesters of lauric (C₁₂) and stearic (Cis) acids.

In some embodiments, the surfactant for the lipid-based drug delivery system can comprise (1) macrogolglycerol hydroxystearate (e.g., Kolliphor RH 40); and (2) polyglyceryl oleate (e.g., Plurol Oleique CC 497 (polyglyceryl-3 dioleate)). When the surfactant for the lipid-based drug delivery system herein comprises macrogolglycerol hydroxystearate and polyglyceryl oleate (e.g., any of those applicable embodiments described herein, such as [12]-[16] and [22]-[37] of the Summary section herein), the weight ratio of macrogolglycerol hydroxystearate to polyglyceryl oleate typically ranges from about 5:1 to 1:5, more typically ranges from about 2:1 to about 1:2, such as about 1.5:1, about 1:1, or about 1:1.5, or any range between the recited values.

In some embodiments, the surfactant for the lipid-based drug delivery system can comprise (1) macrogolglycerol hydroxystearate (e.g., Kolliphor RH 40); (2) polyglyceryl oleate (e.g., Plurol Oleique CC 497 (polyglyceryl-3 dioleate)); and (3) oleoyl polyoxyl-6 glycerides (e.g., Labrafil® M 1944 CS). In such embodiments (e.g., any of those applicable embodiments described herein, such as [12]-[16] and [23]-[37] of the Summary section herein), the weight ratio of macrogolglycerol hydroxystearate to polyglyceryl oleate can range from about 5:1 to 1:5, more typically ranges from about 2:1 to about 1:2, such as about 1.5:1, about 1:1, or about 1:1.5, or any range between the recited values; and the weight ratio of macrogolglycerol hydroxystearate to oleoyl polyoxyl-6 glycerides can also range from about 5:1 to 1:5, more typically ranges from about 2:1 to about 1:2, such as about 1.5:1, about 1:1, or about 1:1.5, or any range between the recited values.

The combination of lipids and surfactants herein is not particularly limited. For example, in some preferred embodiments, the lipid-based drug delivery system comprises (a) medium-chain triglycerides of caprylic (C8) and capric (C10) acids; and (b) macrogolglycerol hydroxystearate (e.g., Kolliphor RH 40). In some embodiments, the lipid-based drug delivery system further comprises one or more lipids selected from triglyceride, monoglyceride, diglyceride, and/or a propylene glycol ester. For example, in some embodiments, the lipid-based drug delivery system further comprises a propylene glycol ester, such as propylene glycol monocaprylate (e.g., Capmul PG-8). In some embodiments, the lipid-based drug delivery system further comprises a mono-, di-, and/or triglyceride, such as a glycerol/glyceryl linoleate (e.g., Maisine® CC, mono-, di- and triglycerides of mainly linoleic (C_{18:2}) and oleic (C_{18:1}) acids, the diester fraction being predominant). In some embodiments, the lipid-based drug delivery system further comprises one or more surfactants described herein. For example, in some embodiments, the lipid-based drug delivery system further comprises a second polyglyceryl ester, such as a polyglyceryl oleate (e.g., Plurol Oleique CC 497 (polyglyceryl-3 dioleate)). In some embodiments, the lipid-based drug delivery system further comprises a polyoxyglyceride, such as oleoyl polyoxyl-6 glycerides (e.g., Labrafil® M 1944 CS).

In some preferred embodiments, the lipid-based drug delivery system comprises (a) medium-chain triglycerides of caprylic (C8) and capric (C10) acids; (b) macrogolglycerol hydroxystearate (e.g., Kolliphor RH 40); and (c) polyglyceryl oleate (e.g., Plurol Oleique CC 497 (polyglyceryl-3 dioleate)). In some embodiments, the lipid-based drug delivery system further comprises one or more lipids selected from triglyceride, monoglyceride, diglyceride, and/or a propylene glycol ester. For example, in some embodiments, the lipid-based drug delivery system further comprises a propylene glycol ester, such as propylene glycol monocaprylate (e.g., Capmul PG-8). In some embodiments, the lipid-based drug delivery system further comprises a mono-, di-, and/or triglyceride, such as a glycerol/glyceryl linoleate (e.g., Maisine® CC, mono-, di- and triglycerides of mainly linoleic (C_{18:2}) and oleic (C_{18:1}) acids, the diester fraction being predominant). In some embodiments, the lipid-based drug delivery system further comprises one or more surfactants described herein. For example, in some embodiments, the lipid-based drug delivery system further comprises a polyoxyglyceride, such as oleoyl polyoxyl-6 glycerides (e.g., Labrafil® M 1944 CS).

In some preferred embodiments, the lipid-based drug delivery system comprises (a) medium-chain triglycerides of caprylic (CS) and capric (C10) acids; (b) macrogolglycerol hydroxystearate (e.g., Kolliphor RH 40); and (c) propylene glycol monocaprylate (e.g., Capmul PG-8). In some embodiments, the lipid-based drug delivery system further comprises one or more surfactants described herein. For example, in some embodiments, the lipid-based drug delivery system further comprises a second polyglyceryl ester, such as a polyglyceryl oleate (e.g., Plurol Oleique CC 497 (polyglyceryl-3 dioleate)). In some embodiments, the lipid-based drug delivery system further comprises a polyoxyglyceride, such as oleoyl polyoxyl-6 glycerides (e.g., Labrafil® M 1944 CS).

In some preferred embodiments, the lipid-based drug delivery system comprises (a) medium-chain triglycerides of caprylic (C8) and capric (C10) acids; (b) macrogolglycerol hydroxystearate (e.g., Kolliphor RH 40); and (c) glycerol/glyceryl linoleate (e.g., Maisine® CC, mono-, di- and triglycerides of mainly linoleic (C_{18:2}) and oleic (C_{18:1}) acids, the diester fraction being predominant). In some embodiments, the lipid-based drug delivery system further comprises one or more surfactants described herein. For example, in some embodiments, the lipid-based drug delivery system further comprises a second polyglyceryl ester, such as a polyglyceryl oleate (e.g., Plurol Oleique CC 497 (polyglyceryl-3 dioleate)). In some embodiments, the lipid-based drug delivery system further comprises a polyoxyglyceride, such as oleoyl polyoxyl-6 glycerides (e.g., Labrafil® M 1944 CS).

In some preferred embodiments, the lipid-based drug delivery system can comprise (a) medium-chain triglycerides of caprylic (C8) and capric (C10) acids; (b) macrogolglycerol hydroxystearate (e.g., Kolliphor RH 40); (c) polyglyceryl oleate (e.g., Plurol Oleique CC 497 (polyglyceryl-3 dioleate)); and (d) oleoyl polyoxyl-6 glycerides (e.g., Labrafil® M 1944 CS).

In some preferred embodiments, the lipid-based drug delivery system can comprise (a) medium-chain triglycerides of caprylic (C8) and capric (C10) acids; (b) macrogolglycerol hydroxystearate (e.g., Kolliphor RH 40); (c) polyglyceryl oleate (e.g., Plurol Oleique CC 497 (polyglyceryl-3 dioleate)); (d) oleoyl polyoxyl-6 glycerides (e.g., Labrafil® M 1944 CS); and (e) propylene glycol monocaprylate (e.g., Capmul PG-8). In some embodiments, the lipid-based drug delivery system can comprise (a) medium-chain triglycerides of caprylic (C8) and capric (C10) acids; (b) macrogolglycerol hydroxystearate (e.g., Kolliphor RH 40); (c) polyglyceryl oleate (e.g., Plurol Oleique CC 497 (polyglyceryl-3 dioleate)); (d) oleoyl polyoxyl-6 glycerides (e.g., Labrafil® M 1944 CS); and (e) propylene glycol monolaurate (e.g., Capmul PG-12, or Lauroglycol™ 90).

In some specific embodiments, the lipid-based drug delivery system can comprise (a) medium-chain triglycerides of caprylic (C8) and capric (C10) acids; (b) macrogolglycerol hydroxystearate (e.g., Kolliphor RH 40); (c) polyglyceryl oleate (e.g., Plurol Oleique CC 497 (polyglyceryl-3 dioleate)); (d) oleoyl polyoxyl-6 glycerides (e.g., Labrafil® M 1944 CS); and (e) glycerol/glyceryl linoleate (e.g., Maisine® CC, mono-, di- and triglycerides of mainly linoleic (C_{18:2}) and oleic (C_{18:1}) acids, the diester fraction being predominant).

The weight percentages of ingredients of the lipid-based drug delivery system are not particularly limited. For example, in some embodiments, the triglyceride, monoglyceride, diglyceride, and/or a propylene glycol ester can be in an amount of about 10-80% (e.g., about 10%, 20%, 30%, 40%, 50%, 60%, 70%, or 80%, or any range between the recited values) by weight of the lipid-based drug delivery system, and the surfactant is in an amount of about 20-90% (e.g., about 20%, 30%, 40%, 50%, 60%, 70%, 80% or 90%, or any range between the recited values, such as about 50-80% or about 40-60%) by weight of the lipid-based drug delivery system.

In some preferred embodiments, the lipid-based drug delivery system comprises (a) medium-chain triglycerides of caprylic (C8) and capric (C10) acids in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system; and (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system. In some embodiments, the lipid-based drug delivery system further comprises one or more lipids selected from triglyceride, monoglyceride, diglyceride, and/or a propylene glycol ester. For example, in some embodiments, the lipid-based drug delivery system further comprises a propylene glycol ester, such as propylene glycol monocaprylate (e.g., Capmul PG-8) in an amount of about 10-40% (e.g., about 10%, about 15%, about 20%, about 25%, about 30%, about 35%, about 40%, or any range between the recited value) by weight of the lipid-based drug delivery system. In some embodiments, the lipid-based drug delivery system further comprises a mono-, di-, and/or triglyceride, such as a glycerol/glyceryl linoleate (e.g., Maisine® CC, mono-, di- and triglycerides of mainly linoleic (C_{18:2}) and oleic (C_{18:1}) acids, the diester fraction being predominant), in an amount of about 10-40% (e.g., about 10%, about 15%, about 20%, about 25%, about 30%, about 35%, about 40%, or any range between the recited value) by weight of the lipid-based drug delivery system. In some embodiments, the lipid-based drug delivery system further comprises one or more surfactants described herein. For example, in some embodiments, the lipid-based drug delivery system further comprises a second polyglyceryl ester, such as a polyglyceryl oleate (e.g., Plurol Oleique CC 497 (polyglyceryl-3 dioleate)) in an amount of about 10-30% (e.g., about 10%, about 15%, about 20%, about 25%, about 30%, or any range between the recited value) by weight of the lipid-based drug delivery system. In some embodiments, the lipid-based drug delivery system further comprises a polyoxyglyceride, such as oleoyl polyoxyl-6 glycerides (e.g., Labrafil® M 1944 CS), in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system.

In some preferred embodiments, the lipid-based drug delivery system comprises (a) medium-chain triglycerides of caprylic (C8) and capric (C10) acids in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system; (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system; and (c) polyglyceryl oleate (e.g., Plurol Oleique CC 497 (polyglyceryl-3 dioleate)) in an amount of about 10-30% (e.g., about 10%, about 15%, about 20%, about 25%, about 30%, or any range between the recited value) by weight of the lipid-based drug delivery system. In some embodiments, the lipid-based drug delivery system further comprises one or more lipids selected from triglyceride, monoglyceride, diglyceride, and/or a propylene glycol ester. For example, in some embodiments, the lipid-based drug delivery system further comprises a propylene glycol ester, such as propylene glycol monocaprylate (e.g., Capmul PG-8) in an amount of about 10-40% (e.g., about 10%, about 15%, about 20%, about 25%, about 30%, about 35%, about 40%, or any range between the recited value) by weight of the lipid-based drug delivery system. In some embodiments, the lipid-based drug delivery system further comprises a mono-, di-, and/or triglyceride, such as a glycerol/glyceryl linoleate (e.g., Maisine® CC, mono-, di- and triglycerides of mainly linoleic (C_{18:2}) and oleic (C_{18:1}) acids, the diester fraction being predominant), in an amount of about 10-40% (e.g., about 10%, about 15%, about 20%, about 25%, about 30%, about 35%, about 40%, or any range between the recited value) by weight of the lipid-based drug delivery system. In some embodiments, the lipid-based drug delivery system further comprises one or more surfactants described herein. For example, in some embodiments, the lipid-based drug delivery system further comprises a polyoxyglyceride, such as oleoyl polyoxyl-6 glycerides (e.g., Labrafil® M 1944 CS), in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system.

In some preferred embodiments, the lipid-based drug delivery system comprises (a) medium-chain triglycerides of caprylic (C8) and capric (C10) acids in an amount of about 10-40% (e.g., about 10%, about 15%, about 20%, about 25%, about 30%, about 35%, about 40%, or any range between the recited value) by weight of the lipid-based drug delivery system; (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system; and (c) propylene glycol monocaprylate (e.g., Capmul PG-8) in an amount of about 10-40% (e.g., about 10%, about 15%, about 20%, about 25%, about 30%, about 35%, about 40%, or any range between the recited value) by weight of the lipid-based drug delivery system. In some embodiments, the lipid-based drug delivery system further comprises one or more surfactants described herein. For example, in some embodiments, the lipid-based drug delivery system further comprises a second polyglyceryl ester, such as a polyglyceryl oleate (e.g., Plurol Oleique CC 497 (polyglyceryl-3 dioleate)) in an amount of about 10-30% (e.g., about 10%, about 15%, about 20%, about 25%, about 30%, or any range between the recited value) by weight of the lipid-based drug delivery system. In some embodiments, the lipid-based drug delivery system further comprises a polyoxyglyceride, such as oleoyl polyoxyl-6 glycerides (e.g., Labrafil® M 1944 CS), in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system.

In some preferred embodiments, the lipid-based drug delivery system comprises (a) medium-chain triglycerides of caprylic (C8) and capric (C10) acids in an amount of about 10-40% (e.g., about 10%, about 15%, about 20%, about 25%, about 30%, about 35%, about 40%, or any range between the recited value) by weight of the lipid-based drug delivery system; (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system; and (c) glycerol/glyceryl linoleate (e.g., Maisine® CC, mono-, di- and triglycerides of mainly linoleic (C_{18:2}) and oleic (C_{18:1}) acids, the diester fraction being predominant), in an amount of about 10-40% (e.g., about 10%, about 15%, about 20%, about 25%, about 30%, about 35%, about 40%, or any range between the recited value) by weight of the lipid-based drug delivery system. In some embodiments, the lipid-based drug delivery system further comprises one or more surfactants described herein. For example, in some embodiments, the lipid-based drug delivery system further comprises a second polyglyceryl ester, such as a polyglyceryl oleate (e.g., Plurol Oleique CC 497 (polyglyceryl-3 dioleate)) in an amount of about 10-30% (e.g., about 10%, about 15%, about 20%, about 25%, about 30%, or any range between the recited value) by weight of the lipid-based drug delivery system. In some embodiments, the lipid-based drug delivery system further comprises a polyoxyglyceride, such as oleoyl polyoxyl-6 glycerides (e.g., Labrafil® M 1944 CS), in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system.

In some preferred embodiments, the lipid-based drug delivery system can comprise (a) medium-chain triglycerides of caprylic (C8) and capric (C10) acids in an amount of about 20-40% (e.g., about 20%, about 25%, about 30%, about 35%, about 40%, or any range between the recited value) by weight of the lipid-based drug delivery system; (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system; (c) polyglyceryl oleate (e.g., Plurol Oleique CC 497 (polyglyceryl-3 dioleate)) in an amount of about 10-30% (e.g., about 10%, about 15%, about 20%, about 25%, about 30%, or any range between the recited value) by weight of the lipid-based drug delivery system; and (d) oleoyl polyoxyl-6 glycerides (e.g., Labrafil® M 1944 CS) in an amount of about 20-40% (e.g., about 20%, about 25%, about 30%, about 35%, about 40%, or any range between the recited value) by weight of the lipid-based drug delivery system.

In some preferred embodiments, the lipid-based drug delivery system can comprise (a) medium-chain triglycerides of caprylic (C8) and capric (C10) acids in an amount of about 10-40% (e.g., about 10%, about 15%, about 20%, about 25%, about 30%, about 35%, about 40%, or any range between the recited value) by weight of the lipid-based drug delivery system; (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system; (c) polyglyceryl oleate (e.g., Plurol Oleique CC 497 (polyglyceryl-3 dioleate)) in an amount of about 10-30% (e.g., about 10%, about 15%, about 20%, about 25%, about 30%, or any range between the recited value) by weight of the lipid-based drug delivery system; (d) oleoyl polyoxyl-6 glycerides (e.g., Labrafil® M 1944 CS) in an amount of about 10-40% (e.g., about 10%, about 15%, about 20%, about 25%, about 30%, about 35%, about 40%, or any range between the recited value) by weight of the lipid-based drug delivery system; and (e) propylene glycol monocaprylate (e.g., Capmul PG-8) and/or propylene glycol monolaurate (e.g., Capmul PG-12, or Lauroglycol™ 90) in an amount of about 10-40% (e.g., about 10%, about 15%, about 20%, about 25%, about 30%, about 35%, about 40%, or any range between the recited value) by weight of the lipid-based drug delivery system.

In some preferred embodiments, the lipid-based drug delivery system can comprise (a) medium-chain triglycerides of caprylic (C8) and capric (C10) acids in an amount of about 10-40% (e.g., about 10%, about 15%, about 20%, about 25%, about 30%, about 35%, about 40%, or any range between the recited value) by weight of the lipid-based drug delivery system; (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system; (c) polyglyceryl oleate (e.g., Plurol Oleique CC 497 (polyglyceryl-3 dioleate)) in an amount of about 10-30% (e.g., about 10%, about 15%, about 20%, about 25%, about 30%, or any range between the recited value) by weight of the lipid-based drug delivery system; (d) oleoyl polyoxyl-6 glycerides (e.g., Labrafil® M 1944 CS) in an amount of about 10-40% (e.g., about 10%, about 15%, about 20%, about 25%, about 30%, about 35%, about 40%, or any range between the recited value) by weight of the lipid-based drug delivery system; and (e) propylene glycol monocaprylate (e.g., Capmul PG-8) in an amount of about 10-40% (e.g., about 10%, about 15%, about 20%, about 25%, about 30%, about 35%, about 40%, or any range between the recited value) by weight of the lipid-based drug delivery system.

In some specific embodiments, the lipid-based drug delivery system can comprise (a) medium-chain triglycerides of caprylic (C8) and capric (C10) acids in an amount of about 10-40% (e.g., about 10%, about 15%, about 20%, about 25%, about 30%, about 35%, about 40%, or any range between the recited value) by weight of the lipid-based drug delivery system; (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system; (c) polyglyceryl oleate (e.g., Plurol Oleique CC 497 (polyglyceryl-3 dioleate)) in an amount of about 10-30% by weight of the lipid-based drug delivery system; (d) oleoyl polyoxyl-6 glycerides (e.g., Labrafil® M 1944 CS) in an amount of about 0-40% (e.g., 0%, about 10%, about 15%, about 20%, about 25%, about 30%, about 35%, about 40%, or any range between the recited value) by weight of the lipid-based drug delivery system; and (e) glycerol/glyceryl linoleate (e.g., Maisine® CC, mono-, di- and triglycerides of mainly linoleic (C_{18:2}) and oleic (C_{18:1}) acids, the diester fraction being predominant) in an amount of about 10-40% (e.g., about 10%, about 15%, about 20%, about 25%, about 30%, about 35%, about 40%, or any range between the recited value) by weight of the lipid-based drug delivery system.

In some specific embodiments, the lipid-based drug delivery system can include any of the vehicles described in the Examples section.

In some embodiments, the lipid-based drug delivery system can have about 20% Kolliphor RH40, about 14% Plurol Oleique CC 497, about 33% Labrafil 1944 CS, and about 33% Labrafac Lipophile WL 1349, by weight of the lipid-based drug delivery system, wherein “about” refers to within 25% of the stated value. For example, about 20% in such embodiments would mean 15% to 25%.

In some embodiments, the lipid-based drug delivery system can have about 20% Kolliphor RH40, about 14% Plurol Oleique CC 497, and about 66% Lauroglycol 90, by weight of the lipid-based drug delivery system, wherein “about” refers to within 25% of the stated value.

In some embodiments, the lipid-based drug delivery system can have about 20% Kolliphor RH40, about 14% Plurol Oleique CC 497, about 16% Labrafil 1944 CS, about 30% Maisine CC, and about 20% Labrafac Lipophile WL 1349, by weight of the lipid-based drug delivery system, wherein “about” refers to within 25% of the stated value.

In some embodiments, the lipid-based drug delivery system can have about 20% Kolliphor RH40, about 14% Plurol Oleique CC 497, about 16% Labrafil 1944 CS, about 30% Capmul PG-8, and about 20% Labrafac Lipophile WL 1349, by weight of the lipid-based drug delivery system, wherein “about” refers to within 25% of the stated value.

It should be understood that when a tradename is used herein, it is meant to include any composition that is within the specification of the product associated with the tradename as of the filing date of this application or if a generic name of such product is available, within the specification of such generic product as of the filing date of this application, such as those specified by various pharmacopeia, including the USP (US Pharmacopeia), European Pharmacopeia (PhEur), Japanese Pharmacopeia, and Chinese Pharmacopeia.

### Abiraterone Decanoate

The pharmaceutical composition herein typically comprises abiraterone decanoate dispersed, such as homogeneously dispersed or dissolved, in the lipid-based drug delivery system herein, with a concentration ranging from about 1 mg/g to about 250 mg/g, about 10 mg/g, about 20 mg/g, about 30 mg/g, about 40 mg/g, about 50 mg/g, about 60 mg/g, about 70 mg/g, about 80 mg/g, about 90 mg/g, about 100 mg/g, about 120 mg/g, about 150 mg/g, about 200 mg/g, about 250 mg/g, or any range between the recited values, e.g., about 10 mg/g to about 150 mg/g, about 20-150 mg/g, about 30-80 mg/g, etc. In some embodiments, the abiraterone decanoate is dissolved in the lipid-based drug delivery system herein.

The abiraterone decanoate is typically present in the pharmaceutical composition herein in its basic form and should be understood as such unless otherwise obvious to the contrary from context. However, in some embodiments, the pharmaceutical composition herein can comprise abiraterone decanoate in its basic form and/or a pharmaceutically acceptable salt thereof.

The abiraterone decanoate for the pharmaceutical composition herein is typically a substantially pure form described herein. For example, the pharmaceutical composition herein typically can be prepared from mixing the substantially pure abiraterone decanoate with the lipid-based drug delivery system and optional other ingredients. In some specific embodiments, the substantially pure abiraterone decanoate is in a crystalline form described herein, preferably, crystalline Form A, and the pharmaceutical composition can be prepared from mixing (e.g., dissolving, suspending, or otherwise forming a mixture) the crystalline form (e.g., Form A) with the lipid-based drug delivery system and optional other ingredients.

In some embodiments, the abiraterone decanoate for the pharmaceutical composition herein is in a substantially pure form, such as having a purity of greater than 80%, preferably greater than 90% (e.g., greater than 95%, greater than 97%, greater than 98%, greater than 99%, greater than 99.5%), by weight, by HPLC area, or both. In some embodiments, the abiraterone decanoate for the pharmaceutical composition herein can be characterized by a purity by weight and/or by HPLC area of about 95%, about 97%, about 99%, about 99.5%, about 99.9%, or any ranges between the specified values. For example, in some embodiments, the abiraterone decanoate for the pharmaceutical composition herein can be characterized by a purity by weight of about 95%, about 97%, about 99%, about 99.5%, about 99.9%, or any ranges between the specified values. In some embodiments, the abiraterone decanoate for the pharmaceutical composition herein can also be characterized as having a low palladium content, such as less than 150 ppm, less than 100 ppm, less than 50 ppm, or less than 10 ppm. In some embodiments, the abiraterone decanoate for the pharmaceutical composition herein conforms to the specification shown in Table 1 herein (see Example 1B). Exemplary procedures for preparing the substantially pure abiraterone decanoate are shown in the Examples section. HPLC methods suitable for measuring the purity of the abiraterone decanoate are also described in the Examples section. The substantially pure abiraterone decanoate can be in a solid form (e.g., a crystalline form described herein, preferably, Form A, amorphous form, or a combination thereof) or in a solution, suspension, or another form. For the avoidance of doubt, the pharmaceutical composition herein comprising the substantially pure abiraterone decanoate and one or more other ingredients (e.g., the pharmaceutical composition according to [33]-[37] in the Summary Section herein) should be understood as a mixture of the substantially pure abiraterone decanoate herein and the one or more other ingredients, for example, such formulation can be obtained directly or indirectly from mixing (e.g., dissolving, suspending, or otherwise forming a mixture) the substantially pure abiraterone decanoate with the one or more other ingredients, such as the lipid-based drug delivery system described herein.

### Substantially Pure Abiraterone Decanoate

In some specific embodiments, the pharmaceutical composition herein comprises a substantially pure abiraterone decanoate, which has the following formula:

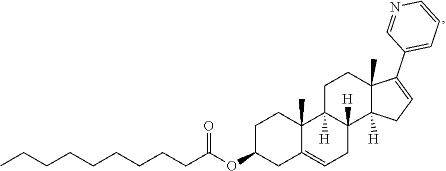

or a pharmaceutically acceptable salt thereof, which is dispersed or dissolved in a lipid-based drug delivery system herein. In some embodiments, the pharmaceutical composition comprises the substantially pure abiraterone decanoate in its basic form, which is dispersed or dissolved in the lipid-based drug delivery system. In some embodiments, the substantially pure abiraterone decanoate has a purity by weight of at least 95%, preferably, at least 98%, such as about 98.5%, about 99%, about 99.5%, or higher. In some embodiments, the substantially pure abiraterone decanoate can be characterized by a purity by weight and/or by HPLC area of about 95%, about 97%, about 99%, about 99.5%, about 99.9%, or any ranges between the specified values. In some embodiments, the substantially pure abiraterone decanoate can be characterized by a purity by weight of about 95%, about 97%, about 99%, about 99.5%, about 99.9%, or any ranges between the specified values. In some embodiments, the substantially pure abiraterone decanoate can also be characterized as having a low palladium content, such as less than 150 ppm, less than 100 ppm, less than 50 ppm, or less than 10 ppm. Abiraterone is typically synthesized with a step of palladium catalyzed cross-coupling reaction. As such, available abiraterone generally has an undesired level of palladium residue, which may be carried into crude abiraterone decanoate product. As described herein, the present disclosure shows that it is possible to reduce the palladium content of abiraterone decanoate to less than 5 ppm, particularly, 3.7 ppm in Example 1B, by using a process of recrystallization with acetone and water as solvents and activated carbon. In some embodiments, the substantially pure abiraterone decanoate conforms to the specification shown in Table 1 herein (see Example 1B). In some embodiments, the substantially pure abiraterone decanoate comprises an impurity derived from ethyl prasterone. For example, in some embodiments, the substantially pure abiraterone decanoate comprises ethyl prasterone decanoate having the formula:

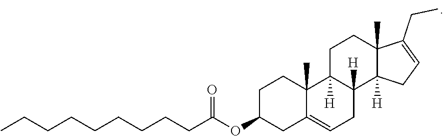

Typically, when present, the substantially pure abiraterone decanoate comprises the ethyl prasterone decanoate in an amount of less than 2% by weight, such as less than 1% by weight, less than 0.5% by weight, such as less than 0.3%, less than 0.2%, or less than 0.1% by weight. The amount of ethyl prasterone decanoate can be readily determined by HPLC methods, such as those descried herein. In some embodiments, the substantially pure abiraterone decanoate can also contain no detectable amount of ethyl prasterone decanoate. Abiraterone starting material is readily available from commercial sources in high purity. Abiraterone starting material obtained from a process using

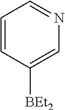

in a cross-coupling reaction to introduce the 3-pyridyl group in abiraterone may contain small amount of impurities which can ultimately be converted into ethyl prasterone. In some embodiments, the substantially pure abiraterone decanoate can be prepared from an abiraterone starting material which has no detectable amount of ethyl prasterone, e.g., those obtained from processes that do not include a cross-coupling with

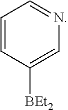

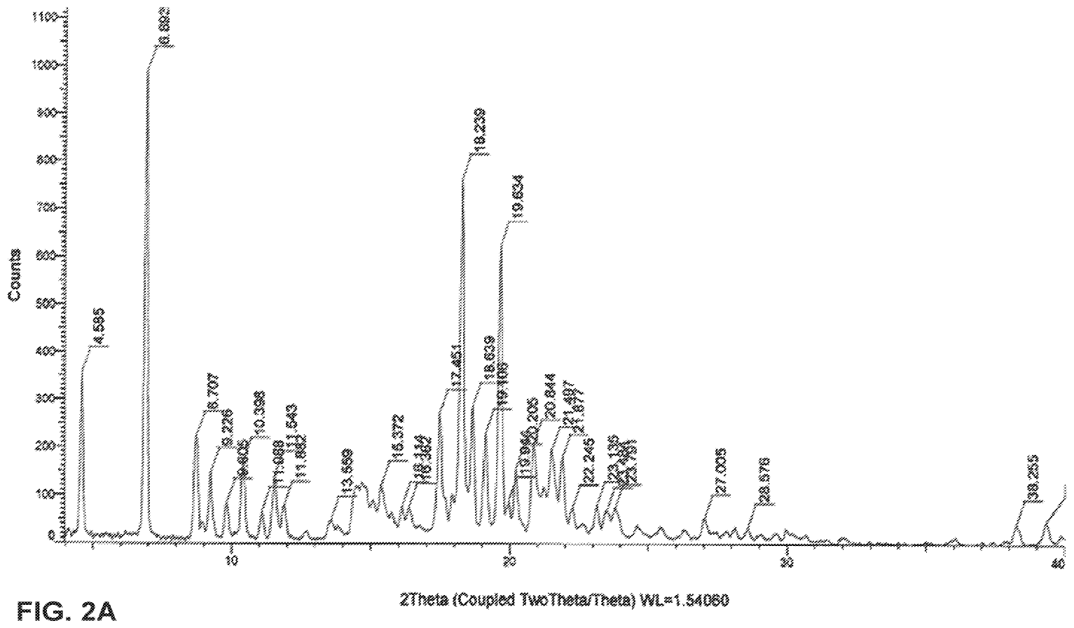

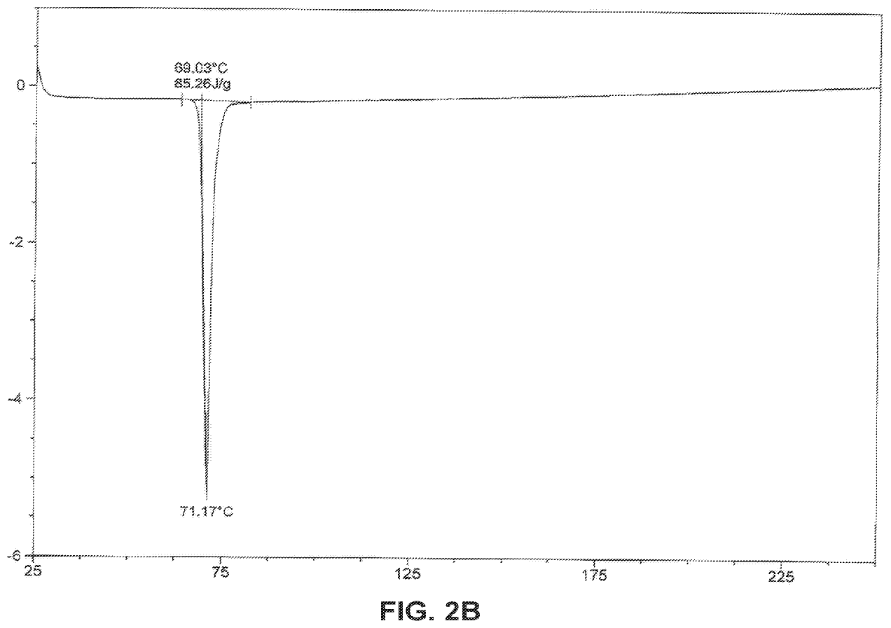

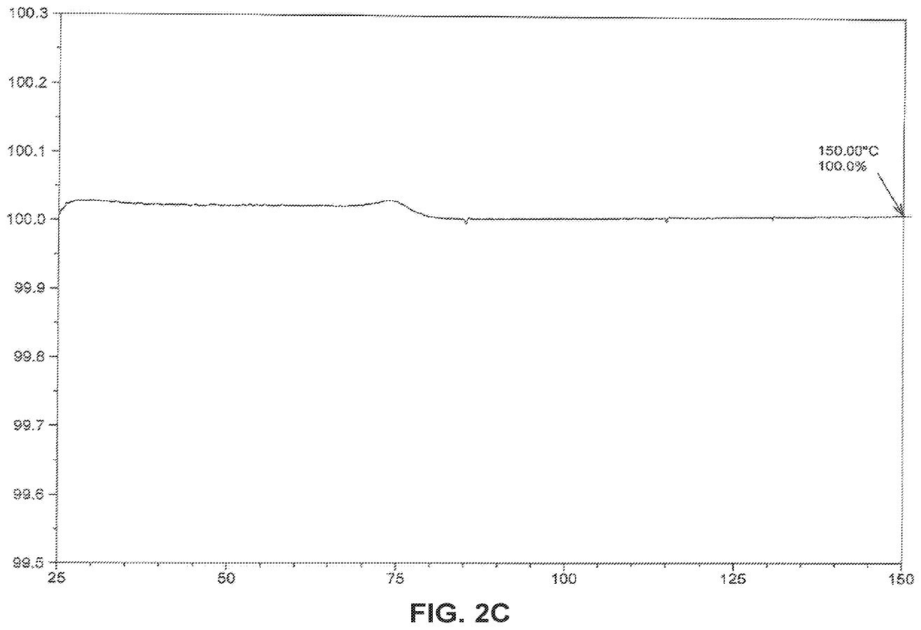

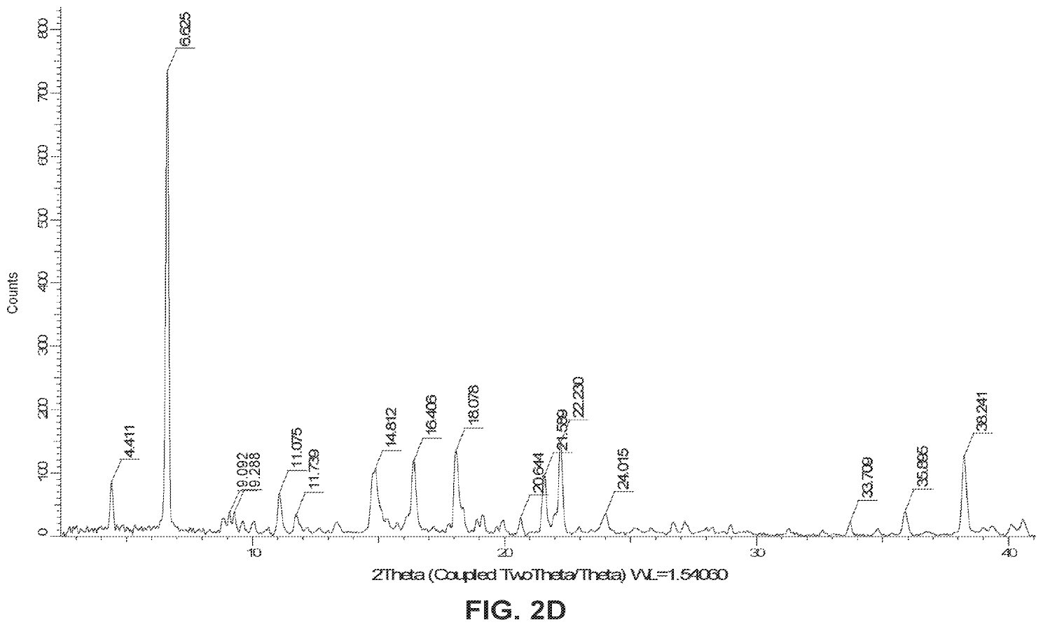

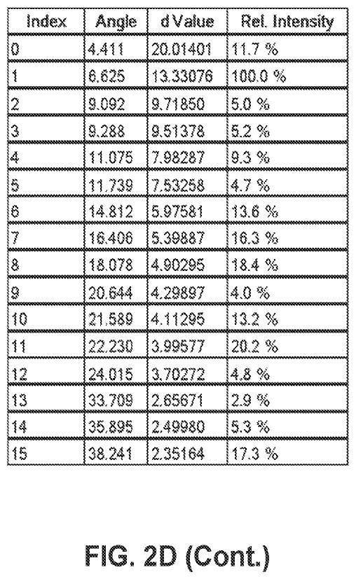

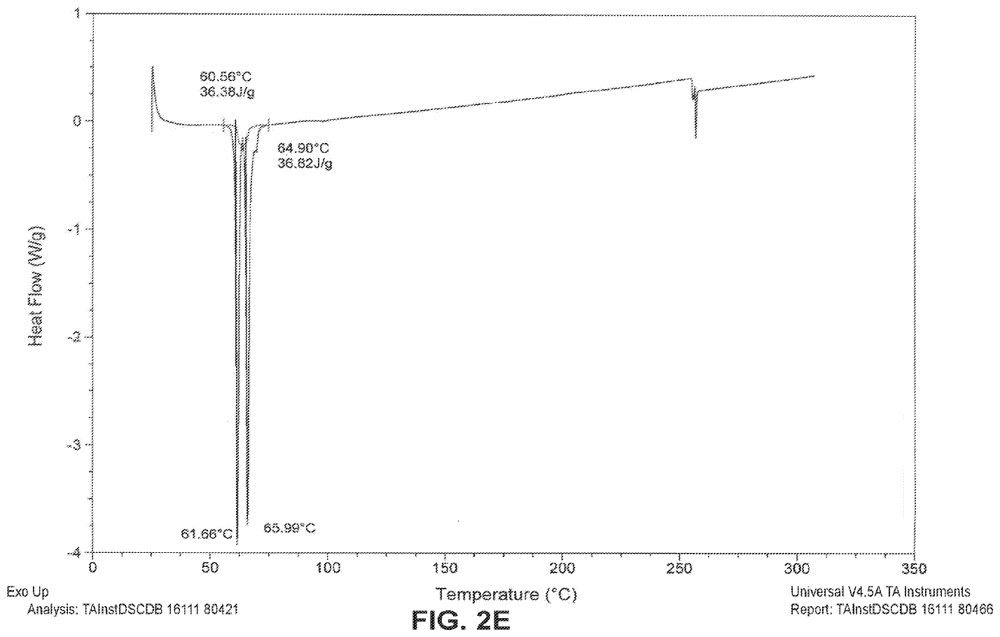

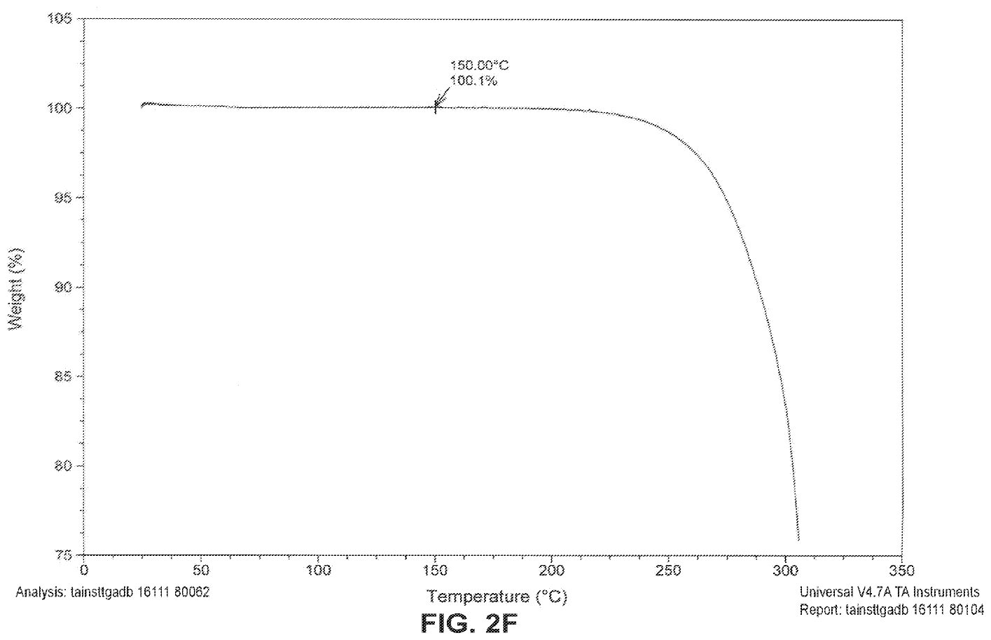

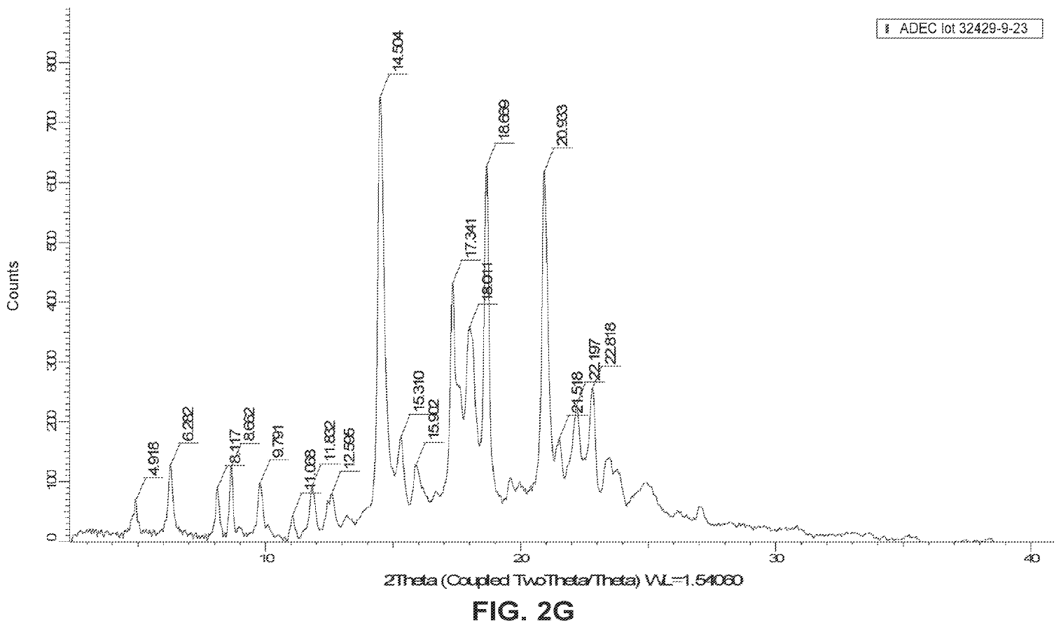

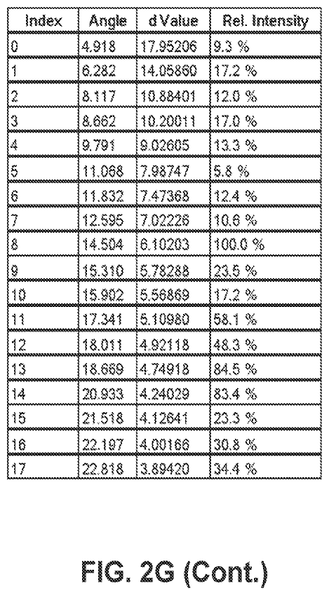

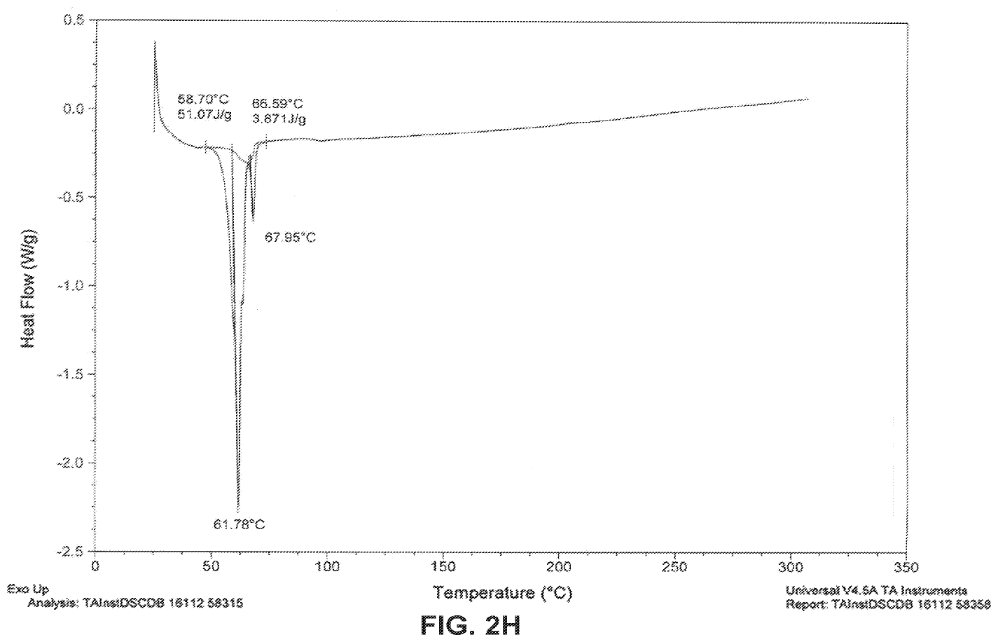

The substantially pure abiraterone decanoate can be in a solid form, such as a crystalline form as described herein. For example, in some embodiments, the substantially pure abiraterone decanoate can be in a crystalline Form A, which can be characterized by an X-Ray Power Diffraction (XRPD) spectrum having one or more (e.g., 1, 2, 3, 4, 5, 6, 7, 8, or 9) of the following peaks: 4.6, 6.9, 8.7, 17.5, 18.3, 18.6, 19.1, 19.6, and 20.8, degrees 2 theta, ±0.2°; a Differential Scanning Calorimetry (DSC) pattern having an endothermic peak with an onset temperature at about 69.0° C.; or a combination thereof. In some embodiments, the crystalline Form A can be characterized by an XRPD spectrum substantially the same as shown in FIG. 2A, for example, the XRPD spectrum shows peaks at the respective diffraction angels (degrees 2 theta, ±0.2°) corresponding to the peaks as shown in FIG. 2A, regardless of their relative intensities. In some embodiments, the crystalline Form A can be characterized by a DSC spectrum substantially the same as shown in FIG. 2B.

The pharmaceutical compositions herein comprising abiraterone decanoate typically are a solution or suspension of the abiraterone decanoate in a suitable vehicle as described herein. Typically, the solution can be prepared by dissolving or suspending one or more of the solid forms of abiraterone decanoate, such as crystalline Form A, B, and/or C, in a suitable vehicle. However, in some embodiments, the pharmaceutical composition can also comprise one or more solid form of abiraterone decanoate. For example, in some embodiments, the pharmaceutical composition can comprise the crystalline Form A described herein. In some embodiments, the pharmaceutical composition can comprise the crystalline Form B described herein. In some embodiments, the pharmaceutical composition can comprise the crystalline Form C described herein.

In some embodiments, the pharmaceutical composition herein can also be prepared from abiraterone decanoate comprising crystalline Form A. In some embodiments, the pharmaceutical composition herein can be prepared from crystalline Form A of abiraterone decanoate, which is substantially free of Form B and Form C of abiraterone decanoate, e.g., no detectable amount of Form B and Form C by XRPD. In some embodiments, the pharmaceutical composition herein can be prepared from crystalline Form A of abiraterone decanoate, which is characterized as substantially pure, for example, the crystalline Form A can be characterized as (1) having a Palladium content of less than 50 ppm, such as less than 10 ppm; (2) having a purity by weight of at least 95%, preferably, at least 98%, such as about 98.5%, about 99%, about 99.5%, or higher; (3) having less than 1% (e.g., less than 0.5% by weight, such as less than 0.3%, less than 0.2%, or less than 0.1%) by weight of ethyl prasterone decanoate having the formula:

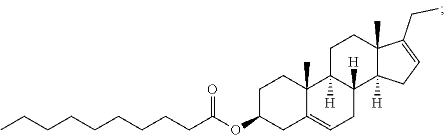

(4) conforming to the specification shown in Table 1, or any combinations thereof.

In some embodiments, the pharmaceutical composition herein can also be prepared from abiraterone decanoate comprising crystalline Form B. In some embodiments, crystalline Form B can be characterized by an X-Ray Power Diffraction (XRPD) spectrum having one or more (e.g., 1, 2, 3, 4, 5, 6, or 7) of the following peaks: 4.4, 6.6, 14.8, 16.4, 18.1, 21.6, and 22.2, degrees 2 theta, ±0.2°; a Differential Scanning Calorimetry (DSC) pattern having two endothermic peaks with onset temperatures at about 60.6° C. and about 64.9° C., respectively; or a combination thereof. In some embodiments, the crystalline Form B can be characterized by an XRPD spectrum substantially the same as shown in FIG. 2D, for example, the XRPD spectrum shows peaks at the respective diffraction angels (degrees 2 theta, ±0.2°) corresponding to the peaks as shown in FIG. 2D, regardless of their relative intensities. In some embodiments, the crystalline Form B can be characterized by a DSC spectrum substantially the same as shown in FIG. 2E. Crystalline Form B can be typically prepared by dissolving abiraterone decanoate in a suitable solvent, such as methanol, ethanol, ethyl acetate, dimethyl acetamide (DMA), methyl tert-butyl ether, 2-propanol, or heptane, to form a solution, and cooling the solution, such as to about −10° C. to about −20° C. to form the crystalline form. Exemplary procedures are shown in Example 1C herein.

In some embodiments, the pharmaceutical composition herein can also be prepared from abiraterone decanoate comprising crystalline Form C. In some embodiments, crystalline Form C can be characterized by an X-Ray Power Diffraction (XRPD) spectrum having one or more of the following peaks: 4.9, 6.3, 14.5, and 15.3, degrees 2 theta, ±0.2°; a Differential Scanning Calorimetry (DSC) pattern having two endothermic peaks with onset temperatures at about 58.7° C. and about 66.6° C., respectively; or a combination thereof. In some embodiments, the crystalline Form C can be characterized by an XRPD spectrum substantially the same as shown in FIG. 2G, for example, the XRPD spectrum shows peaks at the respective diffraction angels (degrees 2 theta, ±0.2°) corresponding to the peaks as shown in FIG. 2G, regardless of their relative intensities. In some embodiments, the crystalline Form C can be characterized by a DSC spectrum substantially the same as shown in FIG. 2H. Crystalline Form C can be typically prepared by dissolving abiraterone decanoate in a suitable solvent, such as 1:1 mixture of ethanol and 2-butanone, and reducing the amount of solvent, such as by evaporation, to form the crystalline form. Exemplary procedures are shown in Example 1C herein.

### Pharmaceutical Compositions Comprising Non-Ionic Surfactants

The pharmaceutical composition herein typically includes abiraterone decanoate dissolved in any of the lipid-based drug delivery system described herein.

In some embodiments, the present disclosure also provides a pharmaceutical composition comprising abiraterone decanoate dissolved in a lipid-based drug delivery system at a concentration ranging from about 10 mg/g to about 150 mg/g (e.g., about 10 mg/g, 20 mg/g, about 30 mg/g, about 40 mg/g, about 50 mg/g, about 60 mg/g, about 70 mg/g, about 80 mg/g, about 90 mg/g, about 100 mg/g, about 120 mg/g, about 150 mg/g, or any range between the recited values, e.g., about 30-80 mg/g or about 30-100 mg/g etc.), wherein the lipid-based drug delivery system comprises (a) a lipid in an amount of about 10-80% by weight of the lipid-based drug delivery system; and (b) one or more non-ionic surfactants in an amount of about 20-90% by weight of the lipid-based drug delivery system, wherein abiraterone decanoate has the following structure:

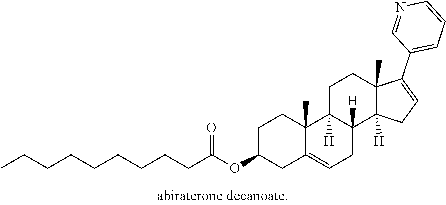

Typically, the weight ratio of the lipid to the one or more non-ionic surfactants ranges from about 5:1 to 1:5, more typically, from about 2:1 to about 1:2, such as about 2:1, about 1.5:1, about 1:1, about 1:1.5, about 1:2, or any range between the recited values.

Suitable lipid is not particularly limited and can include any of the triglyceride, monoglyceride, diglyceride, and/or a propylene glycol ester as described herein.

Suitable non-ionic surfactants are also not particularly limited, which can include any of those described herein.

For example, in some embodiments, the lipid can comprise medium-chain triglycerides of caprylic (C8) and capric (C10) acids (e.g., Labrafac™ lipophile WL 1349). In some embodiments, the lipid can comprise glycerol/glyceryl linoleate (e.g., Maisine® CC). In some embodiments, the lipid can comprise propylene glycol monocaprylate (e.g., Capmul PG-8). In some embodiments, the lipid can comprise propylene glycol monolaurate (e.g., Capmul PG-12, or Lauroglycol™ 90). In some embodiments, the lipid can comprise medium-chain triglycerides of caprylic (C8) and capric (C10) acids (e.g., Labrafac™ lipophile WL 1349) and glycerol/glyceryl linoleate (e.g., Maisine® CC). In some embodiments, the lipid can comprise medium-chain triglycerides of caprylic (C8) and capric (C10) acids (e.g., Labrafac™ lipophile WL 1349) and propylene glycol monocaprylate (e.g., Capmul PG-8). Suitable amounts, ratios, or weight percentages of the medium-chain triglycerides of caprylic (C8) and capric (C10) acids, glycerol/glyceryl linoleate, propylene glycol monocaprylate, and propylene glycol monolaurate include any of those described herein in any combinations.

Typically, the lipid-based drug delivery system comprises two or more, such as 2 or 3, non-ionic surfactants. For example, in some embodiments, the one or more non-ionic surfactants comprise macrogolglycerol hydroxystearate (e.g., Kolliphor RH 40) and/or polyglyceryl oleate (e.g., Plurol Oleique CC 497 (polyglyceryl-3 dioleate)). In some embodiments, the one or more non-ionic surfactants comprise (1) macrogolglycerol hydroxystearate (e.g., Kolliphor RH 40); and (2) polyglyceryl oleate (e.g., Plurol Oleique CC 497 (polyglyceryl-3 dioleate)). In some embodiments, the one or more non-ionic surfactants comprise (1) macrogolglycerol hydroxystearate (e.g., Kolliphor RH 40); (2) polyglyceryl oleate (e.g., Plurol Oleique CC 497 (polyglyceryl-3 dioleate)); and (3) oleoyl polyoxyl-6 glycerides (e.g., Labrafil® M 1944 CS). In some embodiments, the one or more non-ionic surfactants comprise (1) macrogolglycerol hydroxystearate (e.g., Kolliphor RH 40); (2) polyglyceryl oleate (e.g., Plurol Oleique CC 497 (polyglyceryl-3 dioleate)); and (3) lauroyl polyoxyl-6 glycerides (e.g., Labrafil 2130). Suitable amounts, ratios, or weight percentages of the macrogolglycerol hydroxystearate, polyglyceryl oleate, oleoyl polyoxyl-6 glycerides and lauroyl polyoxyl-6 glycerides include any of those described herein in any combinations.

In some specific embodiments, the pharmaceutical composition can comprise abiraterone decanoate dissolved in the lipid-based drug delivery system at a concentration ranging from about 20 mg/g to about 120 mg/g (e.g., about 20 mg/g, about 30 mg/g, about 40 mg/g, about 50 mg/g, about 60 mg/g, about 70 mg/g, about 80 mg/g, about 90 mg/g, about 100 mg/g, about 120 mg/g, or any range between the recited values, e.g., about 30-80 mg/g or about 30-100 mg/g etc.), wherein the lipid-based drug delivery system comprises (a) medium-chain triglycerides of caprylic (C8) and capric (C10) acids in an amount of about 20-40% (e.g., about 20%, about 25%, about 30%, about 35%, about 40%, or any range between the recited value) by weight of the lipid-based drug delivery system; (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system; (c) polyglyceryl oleate (e.g., Plurol Oleique CC 497 (polyglyceryl-3 dioleate)) in an amount of about 10-30% (e.g., about 10%, about 15%, about 20%, about 25%, about 30%, or any range between the recited value) by weight of the lipid-based drug delivery system; and (d) oleoyl polyoxyl-6 glycerides (e.g., Labrafil® M 1944 CS) in an amount of about 20-40% (e.g., about 20%, about 25%, about 30%, about 35%, about 40%, or any range between the recited value) by weight of the lipid-based drug delivery system.

In some specific embodiments, the pharmaceutical composition can comprise abiraterone decanoate dissolved in the lipid-based drug delivery system at a concentration ranging from about 20 mg/g to about 120 mg/g (e.g., about 20 mg/g, about 30 mg/g, about 40 mg/g, about 50 mg/g, about 60 mg/g, about 70 mg/g, about 80 mg/g, about 90 mg/g, about 100 mg/g, about 120 mg/g, or any range between the recited values, e.g., about 30-80 mg/g or about 30-100 mg/g etc.), wherein the lipid-based drug delivery system comprises (a) medium-chain triglycerides of caprylic (C8) and capric (C10) acids in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system; (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system; (c) polyglyceryl oleate (e.g., Plurol Oleique CC 497 (polyglyceryl-3 dioleate)) in an amount of about 10-30% (e.g., about 10%, about 15%, about 20%, about 25%, about 30%, or any range between the recited value) by weight of the lipid-based drug delivery system; (d) oleoyl polyoxyl-6 glycerides (e.g., Labrafil® M 1944 CS) in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system; and (e) propylene glycol monocaprylate (e.g., Capmul PG-8) in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system.

In some specific embodiments, the pharmaceutical composition can comprise abiraterone decanoate dissolved in the lipid-based drug delivery system at a concentration ranging from about 20 mg/g to about 120 mg/g (e.g., about 20 mg/g, about 30 mg/g, about 40 mg/g, about 50 mg/g, about 60 mg/g, about 70 mg/g, about 80 mg/g, about 90 mg/g, about 100 mg/g, about 120 mg/g, or any range between the recited values, e.g., about 30-80 mg/g or about 30-100 mg/g etc.), wherein the lipid-based drug delivery system comprises (a) medium-chain triglycerides of caprylic (C8) and capric (C10) acids in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system; (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system; (c) polyglyceryl oleate (e.g., Plurol Oleique CC 497 (polyglyceryl-3 dioleate)) in an amount of about 10-30% (e.g., about 10%, about 15%, about 20%, about 25%, about 30%, or any range between the recited value) by weight of the lipid-based drug delivery system; (d) oleoyl polyoxyl-6 glycerides (e.g., Labrafil® M 1944 CS) in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system; and (e) propylene glycol monolaurate (e.g., Capmul PG-12, or Lauroglycol™ 90) in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system.

In some specific embodiments, the pharmaceutical composition can comprise abiraterone decanoate dissolved in the lipid-based drug delivery system at a concentration ranging from about 20 mg/g to about 120 mg/g (e.g., about 20 mg/g, about 30 mg/g, about 40 mg/g, about 50 mg/g, about 60 mg/g, about 70 mg/g, about 80 mg/g, about 90 mg/g, about 100 mg/g, about 120 mg/g, or any range between the recited values, e.g., about 30-80 mg/g or about 30-100 mg/g etc.), wherein the lipid-based drug delivery system comprises (a) medium-chain triglycerides of caprylic (C8) and capric (C10) acids in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system; (b) macrogolglycerol hydroxystearate (e.g., Kolliphor RH 40) in an amount of about 10-30% (e.g., about 10%, about 15%, about 20%, about 25%, about 30%, or any range between the recited value) by weight of the lipid-based drug delivery system; (c) polyglyceryl oleate (e.g., Plurol Oleique CC 497 (polyglyceryl-3 dioleate)) in an amount of about 10-30% (e.g., about 10%, about 15%, about 20%, about 25%, about 30%, or any range between the recited value) by weight of the lipid-based drug delivery system; (d) oleoyl polyoxyl-6 glycerides (e.g., Labrafil® M 1944 CS) in an amount of about 0-40% (e.g., 0%, about 10%, about 15%, about 20%, about 25%, about 30%, about 35%, about 40%, or any range between the recited value, such as about 20-40% or 10-30% etc.) by weight of the lipid-based drug delivery system; and (e) glycerol/glyceryl linoleate (e.g., Maisine® CC, mono-, di- and triglycerides of mainly linoleic (C_{18:2}) and oleic (C_{18:1}) acids, the diester fraction being predominant) in an amount of about 10-40% (e.g., about 10%, about 15%, about 20%, about 25%, about 30%, about 35%, about 40%, or any range between the recited value, such as about 20-40% or 10-30% etc.) by weight of the lipid-based drug delivery system.

In some specific embodiments, the pharmaceutical composition can comprise abiraterone decanoate dissolved in a vehicle described in any of the examples herein, see e.g., Examples 2 and 3.

In some embodiments, the pharmaceutical composition can comprise abiraterone decanoate dissolved in a lipid-based drug delivery system having about 20% Kolliphor RH40, about 14% Plurol Oleique CC 497, about 33% Labrafil 1944 CS, and about 33% Labrafac Lipophile WL 1349, by weight of the lipid-based drug delivery system, wherein “about” refers to within 25% of the stated value. The abiraterone decanoate is typically dissolved at a concentration ranging from about 20 mg/g to about 120 mg/g (e.g., about 20 mg/g, about 30 mg/g, about 40 mg/g, about 50 mg/g, about 60 mg/g, about 70 mg/g, about 80 mg/g, about 90 mg/g, about 100 mg/g, about 120 mg/g, or any range between the recited values, e.g., about 30-80 mg/g or about 30-100 mg/g etc.), up to the maximum solubility of abiraterone decanoate in the lipid-based drug delivery system.

In some embodiments, the pharmaceutical composition can comprise abiraterone decanoate dissolved in a lipid-based drug delivery system having about 20% Kolliphor RH40, about 14% Plurol Oleique CC 497, and about 66% Lauroglycol 90, by weight of the lipid-based drug delivery system, wherein “about” refers to within 25% of the stated value. The abiraterone decanoate is typically dissolved at a concentration ranging from about 20 mg/g to about 120 mg/g (e.g., about 20 mg/g, about 30 mg/g, about 40 mg/g, about 50 mg/g, about 60 mg/g, about 70 mg/g, about 80 mg/g, about 90 mg/g, about 100 mg/g, about 120 mg/g, or any range between the recited values, e.g., about 30-80 mg/g or about 30-100 mg/g etc.), up to the maximum solubility of abiraterone decanoate in the lipid-based drug delivery system.

In some embodiments, the pharmaceutical composition can comprise abiraterone decanoate dissolved in a lipid-based drug delivery system having about 20% Kolliphor RH40, about 14% Plurol Oleique CC 497, about 16% Labrafil 1944 CS, about 30% Maisine CC, and about 20% Labrafac Lipophile WL 1349, by weight of the lipid-based drug delivery system, wherein “about” refers to within 25% of the stated value. The abiraterone decanoate is typically dissolved at a concentration ranging from about 20 mg/g to about 120 mg/g (e.g., about 20 mg/g, about 30 mg/g, about 40 mg/g, about 50 mg/g, about 60 mg/g, about 70 mg/g, about 80 mg/g, about 90 mg/g, about 100 mg/g, about 120 mg/g, or any range between the recited values, e.g., about 30-80 mg/g or about 30-100 mg/g etc.), up to the maximum solubility of abiraterone decanoate in the lipid-based drug delivery system.

In some embodiments, the pharmaceutical composition can comprise abiraterone decanoate dissolved in a lipid-based drug delivery system having about 20% Kolliphor RH40, about 14% Plurol Oleique CC 497, about 16% Labrafil 1944 CS, about 30% Capmul PG-8, and about 20% Labrafac Lipophile WL 1349, by weight of the lipid-based drug delivery system, wherein “about” refers to within 25% of the stated value. The abiraterone decanoate is typically dissolved at a concentration ranging from about 20 mg/g to about 120 mg/g (e.g., about 20 mg/g, about 30 mg/g, about 40 mg/g, about 50 mg/g, about 60 mg/g, about 70 mg/g, about 80 mg/g, about 90 mg/g, about 100 mg/g, about 120 mg/g, or any range between the recited values, e.g., about 30-80 mg/g or about 30-100 mg/g etc.), up to the maximum solubility of abiraterone decanoate in the lipid-based drug delivery system.

The pharmaceutical composition can be typically formulated for oral administration, such as in the form of a capsule (e.g., a soft gel capsule).

In any of the embodiments described herein, unless specified or otherwise contradictory from context, the pharmaceutical composition herein can also be characterized by one or more of the following: (1) the pharmaceutical composition is storage stable at room temperature; (2) the recovery of abiraterone decanoate is greater than 50% (e.g., 55%, 60%, 70%, 80%, 90%, or up to 100%, or any range between the recited values) when the pharmaceutical composition is assessed using an in vitro dispersion test; and (3) upon oral administration to a mammal, the pharmaceutical composition is capable of delivering a sufficient amount of abiraterone decanoate to the mammal to achieve a therapeutically effective plasma concentration of abiraterone, e.g., for treating a disease or disorder described herein, such as a prostate cancer described herein. For example, in some embodiments, the pharmaceutical composition is storage stable at room temperature, for example, for 1 month, 3 months, 6 months, or longer. In some embodiments, the pharmaceutical composition comprises a solution at room temperature wherein the solution can remain a solution, i.e., no visible formation of drug and/or excipient crystals/precipitations, after storage at room temperature for 1 month, 3 months, 6 months, or longer. In some embodiments, the pharmaceutical composition is characterized in that the recovery of abiraterone decanoate is greater than 50% (e.g., 55%, 60%, 70%, 80%, 90%, or up to 100%, or any range between the recited values) when the pharmaceutical composition is assessed using an in vitro dispersion test. In some embodiments, the pharmaceutical composition is characterized in that upon oral administration to a mammal, the pharmaceutical composition is capable of delivering a sufficient amount of abiraterone decanoate to the mammal to achieve a therapeutically effective plasma concentration of abiraterone, e.g., for treating a disease or disorder described herein, such as a prostate cancer described herein. In some embodiments, the pharmaceutical composition is characterized in that upon oral administration to a mammal, the pharmaceutical composition is capable of delivering a sufficient amount of abiraterone decanoate to the mammal to achieve aan effective plasma concentration of abiraterone, e.g., for inhibiting CYP17A1.

In any of the embodiments described herein, unless specified or otherwise contradictory from context, the pharmaceutical composition can also be characterized by an oral bioavailability of greater than 30%, e.g., up to 60%, 70% or more, based on abiraterone plasma concentration profile, when tested in rats.

In any of the embodiments described herein, unless specified or otherwise contradictory from context, the pharmaceutical composition can be a self-dispersing drug delivery system, such as a self-emulsifying drug delivery system or self-microemulsifying drug delivery system.

In any of the embodiments described herein, unless specified or otherwise contradictory from context, the pharmaceutical composition can be characterized in that upon oral administration to a mammal, at least a portion of the abiraterone decanoate is absorbed through the lymphatic system.

### Methods of Preparing Pharmaceutical Compositions

In some embodiments, the present disclosure also provides methods of preparing the pharmaceutical compositions comprising the abiraterone decanoate and lipid-based drug delivery system as described herein (e.g., any of those applicable embodiments described herein, such as [1]-[37] of the Summary section herein). The method typically comprises mixing, such as dissolving, the abiraterone decanoate (e.g., any of the substantially pure abiraterone decanoate described herein, a crystalline form of abiraterone decanoate, such as Form A, B, or C) with the lipid-based drug delivery system (e.g., any of those described herein). The particular sequences of mixing is typically not important. For example, although not prohibited, it is not necessary to prepare the lipid-based drug delivery system first before mixing it with abiraterone decanoate. In some embodiments, abiraterone decanoate can be mixed, such as dissolved, with one or more components of the lipid-based drug delivery system before mixing with the other components of the lipid-based drug delivery system. The amounts of abiraterone decanoate and the lipid-based drug delivery system include any of those described herein.

### Emulsions

In some embodiments, the present disclosure also provides emulsions comprising abiraterone decanoate. In some embodiments, the emulsions can also be considered pharmaceutical compositions described herein and can be orally administered to a subject in need.

In some embodiments, the emulsion can comprise (a) abiraterone decanoate; (b) a lipid; and (c) a non-ionic surfactant, wherein the lipid phase of the emulsion comprises abiraterone decanoate dispersed in the lipid, wherein abiraterone decanoate has the following structure:

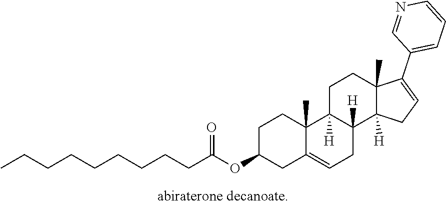

Typically, the weight ratio of the lipid to the non-ionic surfactant ranges from about 5:1 to 1:5, more typically, from about 2:1 to about 1:2, such as about 2:1, about 1.5:1, about 1:1, about 1:1.5, about 1:2, or any range between the recited values.

Suitable lipid is not particularly limited and can include any of the triglyceride, monoglyceride, diglyceride, and/or a propylene glycol ester as described herein.

Suitable non-ionic surfactants are also not particularly limited, which can include any of those described herein.

For example, in some embodiments, the lipid can comprise medium-chain triglycerides of caprylic (C8) and capric (C10) acids (e.g., Labrafac™ lipophile WL 1349). In some embodiments, the lipid can comprise glycerol/glyceryl linoleate (e.g., Maisine® CC). In some embodiments, the lipid can comprise propylene glycol monocaprylate (e.g., Capmul PG-8). In some embodiments, the lipid can comprise propylene glycol monolaurate (e.g., Capmul PG-12, or Lauroglycol™ 90). In some embodiments, the lipid can comprise medium-chain triglycerides of caprylic (C8) and capric (C10) acids (e.g., Labrafac™ lipophile WL 1349) and glycerol/glyceryl linoleate (e.g., Maisine® CC). In some embodiments, the lipid can comprise medium-chain triglycerides of caprylic (C8) and capric (C10) acids (e.g., Labrafac™ lipophile WL 1349) and propylene glycol monocaprylate (e.g., Capmul PG-8). Suitable amounts, ratios, or weight percentages of the medium-chain triglycerides of caprylic (C8) and capric (C10) acids, glycerol/glyceryl linoleate, propylene glycol monocaprylate, and propylene glycol monolaurate include any of those described herein in any combinations.

Typically, the emulsion comprises two or more, such as 2 or 3, non-ionic surfactants. For example, in some embodiments, the non-ionic surfactant comprises macrogolglycerol hydroxystearate (e.g., Kolliphor RH 40) and/or polyglyceryl oleate (e.g., Plurol Oleique CC 497 (polyglyceryl-3 dioleate)). In some embodiments, the non-ionic surfactant comprises (1) macrogolglycerol hydroxystearate (e.g., Kolliphor RH 40); and (2) polyglyceryl oleate (e.g., Plurol Oleique CC 497 (polyglyceryl-3 dioleate)). In some embodiments, the non-ionic surfactant comprises (1) macrogolglycerol hydroxystearate (e.g., Kolliphor RH 40); (2) polyglyceryl oleate (e.g., Plurol Oleique CC 497 (polyglyceryl-3 dioleate)); and (3) oleoyl polyoxyl-6 glycerides (e.g., Labrafil® M 1944 CS). In some embodiments, the non-ionic surfactant comprises (1) macrogolglycerol hydroxystearate (e.g., Kolliphor RH 40); (2) polyglyceryl oleate (e.g., Plurol Oleique CC 497 (polyglyceryl-3 dioleate)); and (3) lauroyl polyoxyl-6 glycerides (e.g., Labrafil 2130). Suitable amounts, ratios, or weight percentages of the macrogolglycerol hydroxystearate, polyglyceryl oleate, oleoyl polyoxyl-6 glycerides and lauroyl polyoxyl-6 glycerides include any of those described herein in any combinations.

In some embodiments, the present disclosure also provides an emulsion produced by mixing the pharmaceutical composition comprising the abiraterone decanoate and lipid-based drug delivery system herein (e.g., any of those described herein, such as [1]-[37] shown in the Summary section herein) with water.

In some embodiments, the present disclosure also provides an emulsion produced by administering the pharmaceutical composition comprising the abiraterone decanoate and lipid-based drug delivery system herein (e.g., any of those described herein, such as [1]-[37] shown in the Summary section herein) to a mammal.

### Pharmaceutical Compositions Comprising Abiraterone Prodrugs

While many of the embodiments herein are directed specifically to abiraterone decanoate, the present disclosure also contemplate oral formulations of abiraterone prodrugs (including abiraterone decanoate) in the lipid-based drug delivery system herein. For example, in some embodiments, the oral abiraterone prodrug formulation can include an abiraterone lipophilic ester, such as an acetate, a propionate, a butanoate, a (vaterate) pentanoate, an isocaproate, a buciclate, a cyclohexanecarboxylate, a phenyl propionate, caproate (hexanoate), an enanthate (heptanoate), a cypionate, an octanoate, a nonanoate, a decanoate, an undecanoate, a dodecanoate, a tridecanoate, a tetradecanoate, a pentadecanoates, or a hexadecanoate of abiraterone in the lipid-based drug delivery system herein.

Other suitable abiraterone prodrugs include any of those described in U.S. Pat. No. 10,792,292 B2 and U.S. Provisional Application Nos. 63/073,502 and 63/149,550, the content of each of which is herein incorporated by reference in its entirety.

For example, in some embodiments, the pharmaceutical composition can include an abiraterone prodrug of Formula I, or a pharmaceutically acceptable salt thereof:

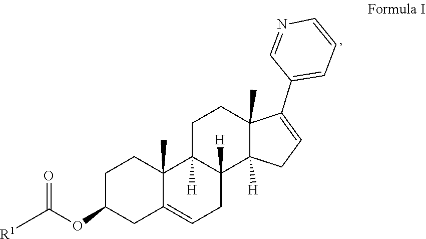

wherein R¹ is R¹⁰, O—R¹⁰, or NHR¹⁰, wherein R¹⁰ is selected from: a C₇₋₃₀ alkyl; C₇₋₃₀ alkenyl; C₇₋₃₀ alkynyl; an alkyl substituted with a cycloalkyl, which typically has a total number of carbons between 5 and 16; an alkyl substituted with a phenyl, which typically has a total number of carbons between 7 and 16; a cycloalkyl optionally substituted with one or more alkyl, which typically has a total number of carbons between 5 and 16; and a branched C5 or C6 alkyl such as

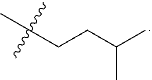 [SMILES: *CCC(C)C]

In some embodiments, R¹⁰ is a C₇₋₃₀ alkyl. As used herein, unless expressly stated to be substituted, an alkyl should be understood as unsubstituted. However, an alkyl can be either linear or branched. In some embodiments, R¹⁰ can be a linear C₇₋₃₀ alkyl. In some embodiments, R¹⁰ can be a branched C₇₋₃₀ alkyl. In some embodiments, R¹⁰ is a linear C₇₋₁₆ alkyl, for example, R¹⁰ can have a formula —(CH₂)ₙ—CH₃, wherein n is an integer between 6 and 15 (e.g., between 6 and 12, such as 6, 7, 8, 9, 10, 11, or 12). In some embodiments, R¹⁰ can be a branched C₇₋₁₆ alkyl.

In some embodiments, R¹⁰ can also be an alkyl substituted with a cycloalkyl. Typically, in such embodiments, R¹⁰ has a total number of carbons between 5 and 16, i.e., the total number of carbons from the alkyl moiety and the cycloalkyl moiety are between 5 and 16. The cycloalkyl typically is unsubstituted. However, in some embodiments, the cycloalkyl can be optionally substituted, e.g., with one or two lower alkyl (e.g, a C₁₋₄ alkyl). In some embodiments, R¹⁰ can be an alkyl substituted with a C₃₋₆ cycloalkyl, which typically has a total number of carbons between 6 and 12. In some embodiments, R¹⁰ can be a linear alkyl substituted with a C₃₋₆ cycloalkyl, for example, R¹⁰ can have a formula —(CH₂)ₙ-Cy, wherein n is an integer of 1-6 (e.g., 1, 2, 3, 4, 5, or 6), and Cy is a C₃₋₆ cycloalkyl (such as cyclopropyl, cyclobutyl, cyclopentyl, or cyclohexyl). In some embodiments, R¹⁰ can have a formula —(CH₂)ₙ-Cy, wherein n is 1 or 2, and Cy is cyclopentyl or cyclohexyl. In some embodiments, R¹⁰ can also be a branched alkyl (e.g., branched C₂₋₆) substituted with a C₃₋₆ cycloalkyl. As used herein, a branched C2 alkyl should be understood as a 1,1-disubstituted ethyl group, for example, —CH(CH₃)—Cy.

In some embodiments, R¹⁰ can also be an alkyl substituted with a phenyl. Typically, in such embodiments, R¹⁰ has a total number of carbons between 7 and 16, i.e., the total number of carbons from the alkyl moiety and the phenyl moiety are between 5 and 16. In some embodiments, R¹⁰ can be a linear alkyl substituted with a phenyl, for example, R¹⁰ can have a formula —(CH₂)ₙ-Cy, wherein n is an integer of 1-6 (e.g., 1, 2, 3, 4, 5, or 6), and Cy is a phenyl. In some embodiments, R¹⁰ can have a formula —(CH₂)ₙ-Cy, wherein n is 1 or 2, and Cy is phenyl. In some embodiments, R¹⁰ can also be a branched alkyl (e.g., branched C₂₋₆) substituted with a phenyl. The phenyl typically is unsubstituted. However, in some embodiments, the phenyl can be optionally substituted, e.g., with one or two lower alkyl (e.g, a C₁₋₄ alkyl).

In some embodiments, R¹⁰ can be a cycloalkyl optionally substituted with one or more alkyl. In such embodiments, R¹⁰ typically has a total number of carbons between 5 and 16, i.e., the total number of carbons of the cycloalkyl and its optional substituents are between 5 and 16. In some embodiments, R¹⁰ can be a C₃₋₆ cycloalkyl, either unsubstituted or substituted with a C₁₋₄ alkyl. In some specific embodiments, R¹⁰ can be

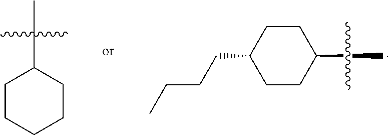

In some embodiments, R¹⁰ can be a branched C5 or C6 alkyl. In some embodiments, R¹⁰ can be

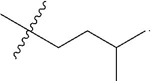 [SMILES: *CCC(C)C]

Other branched C5 or C6 alkyls are also suitable.

In some embodiments, R¹⁰ can be an unsaturated aliphatic group such as a C₇₋₃₀ alkenyl or a C₇₋₃₀ alkynyl.

In some embodiments, the compound of Formula I is an ester of abiraterone, e.g., R¹ is R¹⁰, wherein R¹⁰ is defined herein. In some embodiments, R¹ in Formula I can be a C₇₋₁₆ alkyl, e.g., an alkyl having a formula of —(CH₂)ₙ—CH₃, wherein n is an integer between 6 and 12 (e.g., 6, 7, 8, 9, 10, 11, or 12). In some embodiments, R¹ in Formula I can be represented by the formula —(CH₂)ₙ-Cy, wherein n is an integer of 1-6, and Cy is a C₃₋₆ cycloalkyl or phenyl, for example, in more specific embodiments, n can be 1 or 2, and Cy is cyclopentyl, cyclohexyl, or phenyl. In some specific embodiments, R¹ in Formula I can be

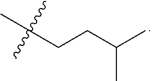 [SMILES: *CCC(C)C]

In some specific embodiments, R¹ in Formula I can be

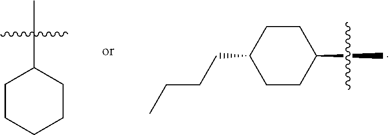

Other suitable groups for R¹ include any of the R¹⁰ defined herein.

In some embodiments, R¹ in Formula I can also be O—R¹⁰ or NHR¹⁰, wherein R¹⁰ is defined herein.

In some embodiments, the pharmaceutical composition can comprise a compound of Formula II, or a pharmaceutically acceptable salt thereof.

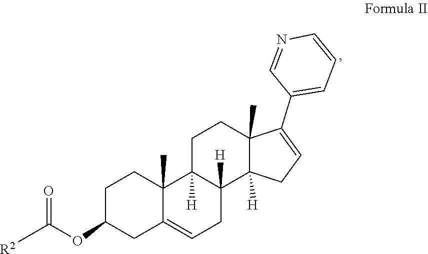

wherein R² is defined herein.

In some embodiments, R² can be selected such that the compound of Formula II is an ester, a carbamate, or a carbonate of abiraterone. In some embodiments, R² is R²⁰, O—R²⁰, or NHR²⁰, and R²⁰ is selected from: a C₁₋₃₀ alkyl; a C₂₋₃₀ alkenyl; a C₂₋₃₀ alkynyl; an alkyl substituted with a cycloalkyl, which typically has a total number of carbons between 4 and 30; an alkyl substituted with a phenyl, which typically has a total number of carbons between 7 and 30; and a cycloalkyl optionally substituted with one or more alkyl, which typically has a total number of carbons between 3 and 30.

In some embodiments, R²⁰ is a C₁₋₆ alkyl. In some embodiments, R²⁰ can be a linear C₁₋₁₆ alkyl. In some embodiments, R²⁰ can be a branched C₃₋₁₆ alkyl. In some embodiments, R²⁰ can be a branched C5 or C6 alkyl. In some embodiments, R²⁰ can be

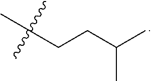 [SMILES: *CCC(C)C]

In some embodiments, R²⁰ can have a formula —(CH₂)ₙ—CH₃, wherein n is an integer between 0 and 12 (e.g., between 6 and 12, such as 6, 7, 8, 9, 10, 11, or 12).

In some embodiments, R²⁰ can also be an alkyl substituted with a cycloalkyl. Typically, in such embodiments, R²⁰ has a total number of carbons between 4 and 30, such as between 5 and 16 (i.e., the total number of carbons from the alkyl moiety and the cycloalkyl moiety are between 5 and 16). The cycloalkyl typically is unsubstituted. However, in some embodiments, the cycloalkyl can be optionally substituted, e.g., with one or two lower alkyl (e.g, a C₁₋₄ alkyl). In some embodiments, R²⁰ can be an alkyl substituted with a C₃₋₆ cycloalkyl, which typically has a total number of carbons between 6 and 12. In some embodiments, R²⁰ can be a linear alkyl substituted with a C₃₋₆ cycloalkyl, for example, R²⁰ can have a formula —(CH₂)ₙ-Cy, wherein n is an integer of 1-6 (e.g., 1, 2, 3, 4, 5, or 6), and Cy is a C₃₋₆ cycloalkyl (such as cyclopropyl, cyclobutyl, cyclopentyl, or cyclohexyl). In some embodiments, R²⁰ can have a formula —(CH₂)ₙ-Cy, wherein n is 1 or 2, and Cy is cyclopentyl or cyclohexyl. In some embodiments, R²⁰ can also be a branched alkyl (e.g., branched C₂₋₆) substituted with a C₃₋₆ cycloalkyl.

In some embodiments, R²⁰ can also be an alkyl substituted with a phenyl. Typically, in such embodiments, R²⁰ has a total number of carbons between 7 and 30, e.g., between 7 and 16 (i.e., the total number of carbons from the alkyl moiety and the phenyl moiety are between 7 and 16). In some embodiments, R²⁰ can be a linear alkyl substituted with a phenyl, for example, R²⁰ can have a formula —(CH₂)ₙ-Cy, wherein n is an integer of 1-6 (e.g., 1, 2, 3, 4, 5, or 6), and Cy is a phenyl. In some embodiments, R²⁰ can have a formula —(CH₂)ₙ-Cy, wherein n is 1 or 2, and Cy is phenyl. In some embodiments, R²⁰ can also be a branched alkyl (e.g., branched C₂₋₆) substituted with a phenyl. The phenyl typically is unsubstituted. However, in some embodiments, the phenyl can be optionally substituted, e.g., with one or two lower alkyl (e.g, a C₁₋₄ alkyl).

In some embodiments, R²⁰ can be a cycloalkyl optionally substituted with one or more alkyl. In such embodiments, R²⁰ typically has a total number of carbons between 3 and 30, e.g., 5 and 16 (i.e., the total number of carbons of the cycloalkyl and its optional substituents are between 5 and 16). In some embodiments, R²⁰ can be a C₃₋₆ cycloalkyl, either unsubstituted or substituted with a C₁₋₄ alkyl. In some specific embodiments, R²⁰ can be

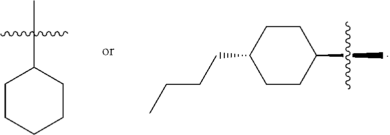

In some embodiments, R²⁰ can be an unsaturated aliphatic group such as a C₂₋₃₀ alkenyl or a C₂₋₃₀ alkynyl.

In some preferred embodiments, the compound of Formula II is an abiraterone ester, e.g., R² is R²⁰, wherein R²⁰ is defined herein. In some embodiments, R² in Formula II can be a C₁₋₁₆ alkyl, e.g., an alkyl having a formula of —(CH₂)ₙ—CH₃, wherein n is an integer between 0 and 12. In some embodiments, R² in Formula II can be represented by the formula —(CH₂)ₙ-Cy, wherein n is an integer of 1-6, and Cy is a C₃₋₆ cycloalkyl or phenyl, for example, in more specific embodiments, n can be 1 or 2, and Cy is cyclopentyl, cyclohexyl, or phenyl. In some specific embodiments, R² in Formula II can be

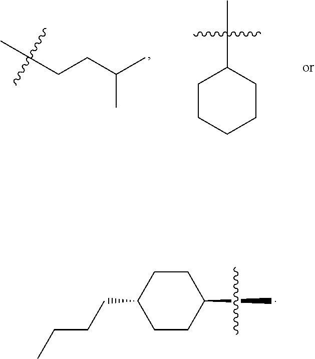

Other suitable groups for R² include any of the R²⁰ defined herein. In some embodiments, the abiraterone ester can be an acetate, a propionate, a butanoate, a (vaterate) pentanoate, an isocaproate, a buciclate, a cyclohexanecarboxylate, a phenyl propionate, caproate (hexanoate), a enanthate (heptanoate), a cypionate, an octanoate, a noncanoate, a decanoate, an undecanoate, a dodecanoate, a tridecanoate, a tetradecanoate, a pentadecanoates, or a hexadecanoate of abiraterone. In some embodiments, the abiraterone ester can be abiraterone acetate, abiraterone propionate, and abiraterone decanoate. In some specific embodiments, the abiraterone ester can be abiraterone pentanoate, abiraterone hexanoate, abiraterone heptanoate, abiraterone decanoate, abiraterone isocaproate, or abiraterone cypionate.

In some embodiments, R² in Formula II can also be O—R²⁰ or NHR²⁰, wherein R²⁰ is defined herein.

Typically, compounds of Formula I or II can be present in a formulation in the basic form. However, in some embodiments, pharmaceutically acceptable salts of compounds of Formula I or II are also useful. Unless specifically referred to as in its salt form or otherwise contradictory from context, the compound of Formula I or II can be in its basic form in the pharmaceutical compositions described herein. In some embodiments, the compound of Formula I or II can be in a substantially pure form.

### Methods of Treatment

In some embodiments, the present disclosure provides a method of treating a disease or disorder described herein in a subject in need thereof. The method typically comprises orally administering to the subject a therapeutically effective amount of pharmaceutical composition herein (e.g., any of those described herein, such as [1]-[37] shown in the Summary section herein) or an emulsion described herein (e.g., any of those described herein, such as [82]-[90] shown in the Summary section herein). Typically, the oral administration delivers a sufficient amount of abiraterone decanoate to the subject to achieve an effective inhibition of CYP17A1 and/or modulation of various steroid hormone levels in the subject, such as androgens, estrogens, glucocorticoids, progesterone, and mineralocorticoids.

U.S. Pat. No. 10,792,292 B2, and U.S. Provisional Application Nos. 63/073,502 and 63/149,550 show various advantages of parenteral administration of abiraterone prodrugs, such as abiraterone decanoate, such as sustained inhibition of CYP17A1, sustained PD effects such as increase of progesterone level and reduction of cortisol, dihydrotestosterone and testosterone levels for up to 70 days or more, sustained reduction of testosterone within a few days following the first administration of the prodrug without the need for castration or another drug that is effective in lowering testosterone levels, and generally well tolerated, for example, no liver toxicity observed from intramuscular administration of abiraterone decanoate at the tested doses. As detailed in Applicant's earlier applications, and without wishing to be bound by theories, the prolonged PD effects observed may in part due to the slow-, tight-binding of CYP17A1 by abiraterone, which may have effectively achieved irreversible inhibition of CYP17A1, see e.g., Cheong E. J. Y., et al. J. Pharmacol. Exp. Ther. 374:438-451 (2020). Also without wishing to be bound by theories, it was believed that the intramuscular administration of the abiraterone prodrug resulted in both a sustained effective blood plasma levels of abiraterone and favorable tissue distribution of abiraterone and abiraterone prodrug, such as to the testes, which may contribute to the observed effects on serum steroids that are not achieved by oral abiraterone acetate formulations (e.g., Zytiga®).

As shown in the Examples section herein, oral administration of exemplary lipid-based formulations of abiraterone decanoate similarly achieved inhibition of CYP17A1 as evidenced by the increase of progesterone level and reduction of testosterone levels for at least 24 hours or more. It is believed that oral administration can achieve similar pharmacodynamics effects as observed in Applicant's earlier intramuscular administration, albeit at a different dosing regimen. Similarly, it is also believed that the methods herein would not rely on castration to achieve a desired testosterone level, thus can also be advantageously used at least for treating subjects who do not wish to be castrated and/or who are sensitive to or otherwise intolerant with gonadal testosterone suppressing drugs. It is further believed that oral administration of abiraterone decanoate would be generally well tolerated, and can be used to treat subjects suffering from hepatic impairment, such as moderate to severe hepatic impairment (Child-Pugh Class B or C), prior to the administering of abiraterone decanoate.

Thus, in some embodiments, the oral formulations herein can be advantageously used for inhibiting CYP17A1 activity, reducing glucocorticoids levels, such as cortisol levels, reducing sex hormone levels such as androgen and/or estrogen levels, and/or treating disorders associated with high glucocorticoids levels, such as cortisol levels, and/or treating disorders due to high sex hormone levels such as androgen and/or estrogen levels.

Accordingly, in some embodiments, the present disclosure provides a method of treating a disease or disorder described herein in a subject in need thereof, the method comprising administering to the subject an effective amount of the pharmaceutical composition herein (e.g., any of those described herein, such as [1]-[37] of the Summary section). In some embodiments, the present disclosure provides a method of treating a disease or disorder described herein in a subject in need thereof, the method comprising administering to the subject an effective amount of an emulsion described herein (e.g., any of those described herein, such as [82]-[90] shown in the Summary section herein).

Various diseases or disorders are suitable to be treated with the methods herein. For example, in some embodiments, the disease or disorder can be a sex hormone-dependent benign or malignant disorder, an androgen receptor drive cancer, a syndrome due to androgen excess, and a syndrome due to glucocorticoid excess, such as a disease or disorder selected from prostate cancer, breast cancer, endometrial cancer, ovarian cancer, bladder cancer, hepatocellular carcinoma, lung cancer, endometriosis, polycystic ovary syndrome, Cushing's syndrome, Cushing's disease, classical or nonclassical congenital adrenal hyperplasia, precocious puberty, hirsutism, and combinations thereof.

In some embodiments, the hormone-dependent benign or malignant disorders can be androgen-dependent disorders and/or estrogen-dependent disorders such as androgen or estrogen-dependent cancers. In some embodiments, the sex hormone-dependent benign or malignant disorder can be prostate cancer or breast cancer. In some embodiments, the sex hormone-dependent benign or malignant disorder is CRPC or CSPC. In some embodiments, the sex hormone-dependent benign or malignant disorder can be metastatic CRPC or metastatic CSPC. In some embodiments, the sex hormone-dependent benign or malignant disorder can also be endometrial cancer, ovarian cancer, bladder cancer, hepatocellular carcinoma, or lung cancer.

Various non-oncologic syndromes due to androgen excess and/or due to glucocorticoid excess such as hypercortisolemia can also be treated with the methods herein, for example, syndromes due to androgen excess such as endometriosis, polycystic ovary syndrome, classical or nonclassical congenital adrenal hyperplasia, precocious puberty, hirsutism, etc., and/or syndromes due to cortisole excess such as Cushing's syndrome, Cushing's disease, etc.

In some specific embodiments, the methods herein are for treating a sex hormone dependent or androgen receptor driven cancer.

In some embodiments, the sex hormone dependent or androgen receptor driven cancer can be androgen receptor positive salivary duct carcinoma, or androgen receptor positive glioblastoma multiforme.

In some embodiments, the sex hormone dependent or androgen receptor driven cancer is prostate cancer (e.g., any of those described herein). Prostate cancer suitable to be treated with the methods herein is not particularly limited and include without limitation any of those prostate cancer for which abiraterone or its derivatives (particularly abiraterone acetate) has been approved for marketing (e.g., in the U.S. or Europe) or for which abiraterone or its derivatives (e.g., abiraterone acetate) is or has been in a clinical trial, such as those trials registered in the website clinicaltrials.gov as of the filing date of this application. For example, in some embodiments, the prostate cancer can be primary/localized prostate cancer (newly diagnosed or early stage), advanced prostate cancer (e.g., after castration for recurrent prostate cancer, locally advanced prostate cancer, etc.), recurrent prostate cancer (e.g., prostate cancer which was not responsive to a primary therapy), non-metastatic castration-resistant prostate cancer, metastatic prostate cancer, metastatic castration-resistant prostate cancer (CRPC), or hormone-sensitive prostate cancer. In some embodiments, the prostate cancer is a localized prostate cancer, e.g., a high risk localized prostate cancer. In some embodiments, the subject having prostate cancer is characterized as having a rising amount of prostate specific antigen, e.g., following radical prostatectomy. In some embodiments, the prostate cancer is a metastatic castration-sensitive prostate cancer, non-metastatic castration-sensitive prostate cancer, non-metastatic castration-resistant prostate cancer, or metastatic castration-resistant prostate cancer. In some embodiments, the prostate cancer is a newly diagnosed high risk metastatic hormone sensitive prostate cancer. In some embodiments, the prostate cancer is a metastatic CRPC (mCRPC), wherein the subject is asymptomatic or mildly symptomatic after failure of androgen deprivation therapy in whom chemotherapy is not yet clinically indicated. In some embodiments, the prostate cancer is a metastatic CRPC (mCRPC), wherein the subject's disease has progressed on or after a taxane-based chemotherapy regimen, such as docetaxel-based or cabazitaxel-based chemotherapy regimen. In some embodiments, the prostate cancer is a refractory prostate cancer. As used herein and unless otherwise specified, the phrase “refractory prostate cancer” means prostate cancer that is not responding to an anti-cancer treatment or prostate cancer that is not responding sufficiently to an anti-cancer treatment. Refractory prostate cancer can also include recurring or relapsing prostate cancer. As used herein and unless otherwise specified, the phrase “relapsing prostate cancer” means prostate cancer that was at one time responsive to an anti-cancer treatment but has become no longer responsive to such treatment or is no longer responding sufficiently to such treatment. As used herein and unless otherwise specified, the phrase “recurring (or recurrent) prostate cancer” means prostate cancer that has returned after a patient has been earlier diagnosed with prostate cancer, undergone treatment or had been previously diagnosed as cancer-free.

In some embodiments, the methods herein can also be used for treating breast cancer. Breast cancer suitable to be treated with the methods herein is not particularly limited. For example, in some embodiments, the breast cancer can be molecular apocrine HER2-negative breast cancer, metastatic breast cancer, such as ER+ metastatic breast cancer, ER+ and HER2 negative breast cancer, AR+ triple negative breast cancer, etc.

In some embodiments, a disease or disorder is associated with 21-hydroxylase deficiency can also be treated with the methods herein.

In some embodiments, the methods herein can be used for treating subjects having a cancer, such as prostate cancer, breast cancer, adrenal cancer, leukemia, lymphoma, myeloma, Waldenström's macroglobulinemia, monoclonal gammopathy, benign monoclonal gammopathy, heavy chain disease, bone and connective tissue sarcoma, brain tumors, thyroid cancer, pancreatic cancer, pituitary cancer, eye cancer, vaginal cancer, vulvar cancer, cervical cancer, uterine cancer, endometrial cancer, ovarian cancer, esophageal cancer, stomach cancer, colon cancer, rectal cancer, liver cancer, gallbladder cancer, cholangiocarcinoma, lung cancer, testicular cancer, penal cancer, oral cancer, skin cancer, kidney cancers, Wilms' tumor and bladder cancer.

In some embodiments, the method herein can include treating the subject with one or more additional therapies. For example, in some embodiments, the subject is further treated a radiation therapy. In some embodiments, the method is for treating prostate cancer and includes a combination therapy, which further comprises administering to the subject one or more additional therapies, e.g., as described herein under the section titled Combination Treatment for Prostate Cancer as described herein below. Non-limiting examples of useful additional therapies also include any of those described in [50]-[54] and [61]-[71], [73]-[75], and [77] in the Summary section herein.

Subjects suitable to be treated by the methods herein are not particularly limited, which include subjects at various stages of diseases or treatments and other characteristics. For example, in some embodiments, the subject can be a non-castrated subject. In some embodiments, the subject can be a castrated subject. In some embodiments, the methods herein can also administer the pharmaceutical composition herein (e.g., any of [1]-[37] of the Summary section herein) to the subject without regard to whether the subject is castrated or not. In some embodiments, the subject has not undergone a prostatectomy. In some embodiments, the subject can be characterized as suffering from hepatic impairment, such as moderate to severe hepatic impairment (Child-Pugh Class B or C), prior to the administering of the abiraterone prodrug. In some embodiments, the subject can be characterized as being sensitive to or otherwise intolerant with a gonadotropin-releasing hormone antagonist and/or agonist. In some embodiments, the subject can be characterized as chemotherapy naïve or hormone therapy naïve prior to being administered the pharmaceutical composition herein. However, in some embodiments, the subject can also be treated with chemotherapy or hormone therapy prior to being administered the pharmaceutical composition herein. For example, in some embodiments, the subject can have a disease or disorder (e.g., prostate cancer) that has progressed on or after the chemotherapy and/or hormone therapy, such as a taxane-based chemotherapy regimen, for example, docetaxel-based or cabazitaxel-based chemotherapy. In any of the embodiments described herein, unless directly contradictory, the subject can be a human subject.

Suitable pharmaceutical compositions for the methods herein are not particularly limited and include any of those described herein, such as any of the abiraterone decanoate formulations described herein, e.g., any of those described in the Summary section. Typically, the pharmaceutical composition can be formulated to deliver a therapeutically effective plasma levels of abiraterone over an extended period of time (e.g., at least 1 day, at least 2 days, at least 3 days, etc.) in the subject, following a single oral administration. In some embodiments, the therapeutically effective plasma concentration of abiraterone can be a concentration of at least 1 ng/ml, e.g., at least 2 ng/ml, at least 4 ng/ml, at least 8 ng/ml. In some embodiments, the therapeutically effective blood plasma concentration of abiraterone can also be about 0.5 ng/ml or higher. In some embodiments, the therapeutically effective blood plasma concentration of abiraterone can also be about 0.1 ng/ml or higher. The pharmaceutical composition can be administered to the subject with or without food.

Dosing amounts and frequencies for the methods herein are also not particularly limited and include any of those described herein. Generally, the pharmaceutical composition is administered to the subject ranging from once a day to once a week, such as once a day or once every two or three days. The dosing amounts of the abiraterone decanoate for each administration can vary, typically ranging from 0.5 mg/kg to 200 mg/kg, such as about 0.5 mg/kg to about 200 mg/kg of body weight of a subject.

### Methods of Reducing Steroid Hormone Levels

Some embodiments of the present disclosure are directed to methods of reducing serum steroid hormone level in a subject in need thereof.

In some particular embodiments, the present disclosure provides a method of reducing serum testosterone level in a subject in need thereof, the method comprising orally administering to the subject a pharmaceutical composition herein (e.g., any of those described herein, such as [1]-[37] of the Summary section herein) or an emulsion described herein (e.g., any of those described herein, such as [82]-[90] shown in the Summary section herein).

Subjects suitable to be treated with the methods herein for reducing serum testosterone levels are not particularly limited. For example, in some embodiments, the subject can be a non-castrated subject. In some embodiments, the subject can be a castrated subject. In some embodiments, the methods herein can also administer the pharmaceutical composition herein (e.g., any of those described herein, such as [1]-[37] of the Summary section herein) to the subject without regard to whether the subject is castrated or not. In some embodiments, another drug that is effective in lowering serum and/or gonadal testosterone level, is not administered to the subject concurrently with the administration of the abiraterone prodrug, during the treatment with the abiraterone prodrug, or otherwise interfering with the treatment with the abiraterone prodrug. For example, in some embodiment, the subject is not treated with a gonadal testosterone suppressing drug, other than the administered abiraterone prodrug, in an amount effective to reduce serum testosterone level in the subject. In some embodiment, the subject is not treated with a gonadotropin-releasing hormone antagonist and/or agonist in an amount effective to reduce serum testosterone level in the subject. In some embodiment, the subject is not treated with any gonadal testosterone suppressing drug other than the administered abiraterone prodrug. In some embodiments, the subject is not treated with any gonadotropin-releasing hormone antagonist and/or agonist. In some embodiments, the subject is not treated with a drug selected from buserelin, leuprolide, deslorelin, fertirelin, histrelin, gonadorelin, lecirelin, goserelin, nafarelin, peforelin and triptorelin. In some embodiments, the subject is not treated with a drug selected from abarelix, cetrorelix, degarelix, ganirelix, elagolix, linzagolixa, and relugolix. In some embodiments, the subject can be sensitive to or otherwise intolerant with a gonadotropin-releasing hormone antagonist and/or agonist. In some embodiments, the subject can also be treated with a gonadotropin-releasing hormone antagonist and/or agonist, e.g., described herein.

The subject in need of reduction of testosterone typically suffers from one or more diseases or disorders mediated or associated with androgens. For example, in some embodiments, the subject is characterized as having a sex hormone dependent cancer or androgen receptor driven cancer, e.g., any of those described herein. In some embodiments, the subject is characterized as having androgen receptor positive salivary duct carcinoma, or androgen receptor positive glioblastoma multiforme. In some embodiments, the subject is characterized as having prostate cancer (e.g., any of those described herein). For example, in some embodiments, the prostate cancer is a localized prostate cancer, e.g., a high risk localized prostate cancer. In some embodiments, the subject has not undergone a prostatectomy. In some embodiments, the subject is further treated with a radiation therapy.

In some embodiments, the present disclosure also provides a method of inhibiting CYP17A1 activity such as inhibiting 17α-hydroxylase activity and 17,20-lyase activity, the method comprising administering to a subject in need thereof any of the pharmaceutical composition herein (e.g., any of [1]-[37] of the Summary section herein). In some embodiments, the present disclosure provides a method of inhibiting CYP17A1 activity such as inhibiting 17α-hydroxylase activity and 17,20-lyase activity, the method comprising administering to a subject in need thereof an emulsion described herein (e.g., any of those described herein, such as [82]-[90] shown in the Summary section herein). In some embodiments, the subject suffers from a sex hormone-dependent benign or malignant disorder, e.g., as described herein. In some embodiments, the subject suffers from a syndrome due to androgen excess and/or a syndrome due to glucocorticoid excess such as hypercortisolemia, e.g., as described herein. In some embodiments, the subject suffers from a sex hormone dependent cancer or androgen receptor driven cancer described herein. Suitable pharmaceutical compositions, subjects, dosing regimen, and routes of administrations for the method include any of those described herein in any combination, such as any of those described in connection with the methods shown in the Summary section herein.

In some embodiments, the present disclosure provides a method of reducing the level of glucocorticoids (e.g., cortisol) in a subject in need thereof, the method comprising administering to the subject any of the pharmaceutical composition herein (e.g., any of [1]-[37] of the Summary section herein). In some embodiments, the present disclosure provides a method of reducing the level of glucocorticoids (e.g., cortisol) in a subject in need thereof, the method comprising administering to the subject an emulsion described herein (e.g., any of those described herein, such as [82]-[90] shown in the Summary section herein). In some embodiments, the subject suffers from a syndrome due to glucocorticoid excess such as hypercortisolemia as described herein, such as Cushing's syndrome or Cushing's disease. Suitable pharmaceutical compositions, subjects, dosing regimen, and routes of administrations for the method include any of those described herein in any combination, such as any of those described in connection with the methods shown in the Summary section herein.

In some embodiments, the present disclosure provides a method of reducing the level of androgens (e.g., testosterone) and/or estrogens in a subject in need thereof, the method comprising administering to the subject any of the pharmaceutical composition herein (e.g., any of [1]-[37] of the Summary section herein). In some embodiments, the present disclosure provides a method of reducing the level of androgens (e.g., testosterone) and/or estrogens in a subject in need thereof, the method comprising administering to the subject an emulsion described herein (e.g., any of those described herein, such as [82]-[90] shown in the Summary section herein). In some embodiments, the subject suffers from an androgen receptor driven cancer. In some embodiments, the subject suffers from a syndrome due to androgen excess, such as congenital adrenal hyperplasia (e.g., classical or nonclassical congenital adrenal hyperplasia), endometriosis, polycystic ovary syndrome precocious puberty, hirsutism, etc. In some embodiments, the subject suffers from an androgen and/or estrogen associated cancer, such as prostate cancer or breast cancer. In some embodiments, the subject suffers from a sex hormone dependent cancer described herein. Suitable pharmaceutical compositions, subjects, dosing regimen, and routes of administrations for the method include any of those described herein in any combination, such as any of those described in connection with the methods shown in the Summary section herein.

The abiraterone decanoate in the pharmaceutical composition or emulsion is typically included in a therapeutically effective amount for treating a disease or disorder described herein, such as prostate cancer. In some embodiments, the abiraterone decanoate can be present in the pharmaceutical composition or emulsion in an amount suitable for a dosing frequency ranging from once a day to once a week, such as once a day or once every two or three days, for oral administration to a subject having a sex hormone-dependent benign or malignant disorder, an androgen receptor driven cancer, a syndrome due to androgen excess, and/or a syndrome due to glucocorticoid excess such as hypercortisolemia.

### Combination Treatment

In some embodiments, the methods herein can comprise administering one or more other drug or agent (for example, another cancer chemotherapeutic drug, hormone replacement drug, or hormone ablation drug) to the subject, either concurrently or sequentially, through the same route or a different route of administration. In some embodiments, the other drug or agent can be a steroid, such as prednisone, prednisolone, and/or methylprednisolone. In some embodiments, the other drug or agent can be a chemotherapy drug, such as paclitaxel, mitoxantrone, and/or docetaxel. In some embodiments of the methods herein, the other agent or drug can be a GnRH agonist, such as Leuprolide, deslorelin, goserelin, or triptorelin, e.g., leuprolide acetate (e.g., a long-acting IM injectable formulation). In some embodiments, the other agent or drug can be seocalcitol, bicalutamide, flutamide, a glucocorticoid including, but not limited to, hydrocortisone, prednisone, prednisolone, or dexamethasone. The amount of the other drugs or agents to be administered can vary, typically can be an amount that is effective in treating the respective disease or disorder (e.g., prostate cancer) either alone or in combination with the pharmaceutical composition herein (e.g., any of [1]-[37] of the Summary section herein).

Additional suitable other drugs or agents include those described herein. For example, useful other drugs or agents include, but are not limited to, anticancer agents, hormone ablation agents, anti-androgen agents, differentiating agents, anti-neoplastic agents, kinase inhibitors, anti-metabolite agents, alkylating agents, antibiotic agents, immunological agents, interferon-type agents, intercalating agents, growth factor inhibitors, cell cycle inhibitors, enzymes, topoisomerase inhibitors, biological response modifiers, mitotic inhibitors, matrix metalloprotease inhibitors, genetic therapeutics, and anti-androgens.

For example, suitable anti-cancer agents, including but not limited to, acemannan, aclarubicin, aldesleukin, alemtuzumab, alitretinoin, altretamine, amifostine, amsacrine, anagrelide, anastrozole, ancestim, bexarotene, broxuridine, capecitabine, celmoleukin, cetrorelix, cladribine, clotrimazole, daclizumab, dexrazoxane, dilazep, docosanol, doxifluridine, bromocriptine, carmustine, cytarabine, diclofenac, edelfosine, edrecolomab, eflornithine, emitefur, exemestane, exisulind, fadrozole, filgrastim, finasteride, fludarabine phosphate, formestane, fotemustine, gallium nitrate, gemcitabine, glycopine, heptaplatin, ibandronic acid, imiquimod, iobenguane, irinotecan, irsogladine, lanreotide, leflunomide, lenograstim, lentinan sulfate, letrozole, liarozole, lobaplatin, lonidamine, masoprocol, melarsoprol, metoclopramide, mifepristone, miltefosine, mirimostim, mitoguazone, mitolactol, molgramostim, nafarelin, nartograstim, nedaplatin, nilutamide, noscapine, oprelvekin, osaterone, oxaliplatin, pamidronic acid, pegaspargase, pentosan polysulfate sodium, pentostatin, picibanil, pirarubicin, porfimer sodium, raloxifene, raltitrexed, rasburicase, rituximab, romurtide, sargramostim, sizofiran, sobuzoxane, sonermin, suramin, tasonermin, tazarotene, tegafur, temoporfin, temozolomide, teniposide, tetrachlorodecaoxide, thalidomide, thymalfasin, thyrotropin alfa, topotecan, toremifene, trastuzumab, treosulfan, tretinoin, trilostane, trimetrexate, ubenimex, valrubicin, verteporfin, vinorelbine. Suitable anti-androgen agents include but are not limited to bicalutamide, flutamide and nilutamide. Suitable differentiating agents include, but are not limited to, polyamine inhibitors; vitamin D and its analogs, such as, calcitriol, doxercalciferol and seocalcitol; metabolites of vitamin A, such as, ATRA, retinoic acid, retinoids; short-chain fatty acids; phenylbutyrate; and nonsteroidal anti-inflammatory agents. anti-neoplastic agent, including, but not limited to, tubulin interacting agents, topoisomerase inhibitors and agents, acitretin, alstonine, amonafide, amphethinile, amsacrine, ankinomycin, anti-neoplaston, aphidicolin glycinate, asparaginase, baccharin, batracylin, benfluron, benzotript, bromofosfamide, caracemide, carmethizole hydrochloride, chlorsulfaquinoxalone, clanfenur, claviridenone, crisnatol, curaderm, cytarabine, cytocytin, dacarbazine, datelliptinium, dihaematoporphyrin ether, dihydrolenperone, dinaline, distamycin, docetaxel, elliprabin, elliptinium acetate, epothilones, ergotamine, etoposide, etretinate, fenretinide, gallium nitrate, genkwadaphnin, hexadecylphosphocholine, homoharringtonine, hydroxyurea, ilmofosine, isoglutamine, isotretinoin, leukoregulin, lonidamine, merbarone, merocyanlne derivatives, methylanilinoacridine, minactivin, mitonafide, mitoquidone, mitoxantrone, mopidamol, motretinide, N-(retinoyl)amino acids, N-acylated-dehydroalanines, nafazatrom, nocodazole derivative, ocreotide, oquizanocinc, paclitaxel, pancratistatin, pazelliptine, piroxantrone, polyhaematoporphyrin, polypreic acid, probimane, procarbazine, proglumide, razoxane, retelliptine, spatol, spirocyclopropane derivatives, spirogermanium, strypoldinone, superoxide dismutase, teniposide, thaliblastine, tocotrienol, topotecan, ukrain, vinblastine sulfate, vincristine, vindesine, vinestramide, vinorelbine, vintriptol, vinzolidine, and withanolides. a kinase inhibitor including p38 inhibitors and CDK inhibitors, TNF inhibitors, metallomatrix proteases inhibitors (MMP), COX-2 inhibitors including celecoxib, rofecoxib, parecoxib, valdecoxib, and etoricoxib, SOD mimics or αᵥβ₃ inhibitors. Suitable anti-metabolite agents may be selected from, but not limited to, 5-FU-fibrinogen, acanthifolic acid, aminothiadiazole, brequinar sodium, carmofur, cyclopentyl cytosine, cytarabine phosphate stearate, cytarabine conjugates, dezaguanine, dideoxycytidine, dideoxyguanosine, didox, doxifluridine, fazarabine, floxuridine, fludarabine phosphate, 5-fluorouracil, N-(2′-furanidyl)-5-fluorouracil, isopropyl pyrrolizine, methobenzaprim, methotrexate, norspermidine, pentostatin, piritrexim, plicamycin, thioguanine, tiazofurin, trimetrexate, tyrosine kinase inhibitors, and uricytin. Suitable alkylating agents may be selected from, but not limited to, aldo-phosphamide analogues, altretamine, anaxirone, bestrabucil, budotitane, carboplatin, carmustine, chlorambucil, cisplatin, cyclophosphamide, cyplatate, diphenylspiromustine, diplatinum cytostatic, elmustine, estramustine phosphate sodium, fotemustine, hepsul-fam, ifosfamide, iproplatin, lomustine, mafosfamide, mitolactol, oxaliplatin, prednimustine, ranimustine, semustine, spiromustine, tauromustine, temozolomide, teroxirone, tetraplatin and trimelamol. Suitable antibiotic agents may be selected from, but not limited to, aclarubicin, actinomycin D, actinoplanone, adriamycin, aeroplysinin derivative, amrubicin, anthracycline, azino-mycin-A, bisucaberin, bleomycin sulfate, bryostatin-1, calichemycin, chromoximycin, dactinomycin, daunorubicin, ditrisarubicin B, dexamethasone, doxorubicin, doxorubicin-fibrinogen, elsamicin-A, epirubicin, erbstatin, esorubicin, esperamicin-A1, esperamicin-Alb, fostriecin, glidobactin, gregatin-A, grincamycin, herbimycin, corticosteroids such as hydrocortisone, idarubicin, illudins, kazusamycin, kesarirhodins, menogaril, mitomycin, neoenactin, oxalysine, oxaunomycin, peplomycin, pilatin, pirarubicin, porothramycin, prednisone, prednisolone, pyrindanycin A, rapamycin, rhizoxin, rodorubicin, sibanomicin, siwenmycin, sorangicin-A, sparsomycin, talisomycin, terpentecin, thrazine, tricrozarin A, and zorubicin. Non-limiting examples of suitable steroids include hydrocortisone, prednisone, prednisolone, or dexamethasone.

### Combination Treatment for Prostate Cancer

Prostate cancer treatments often involve multiple therapies, including for example, radiotherapy, surgery, androgen deprivation therapy, hormone therapy, chemotherapy, immunotherapy, and various drug combinations. A search in the website clinicaltrials.gov identified more than 250 clinical trials with abiraterone/abiraterone acetate listed as an intervention agent, and many of such clinical trials include a combination therapy for treating prostate cancer. The pharmaceutical compositions herein (e.g., any of those described herein, such as any of [1]-[37] of the Summary section herein) can also be advantageously used in various combination therapies to replace or supplement the oral administration of abiraterone acetate.

In embodiments where the method treats a non-castrated subject, the methods herein can include the combination treatment that does not treat the subject with a gonadal testosterone suppressing drug, other than the administered abiraterone prodrug, in an amount effective to reduce serum testosterone level in the subject. For example, in some embodiments, the methods herein can include the combination treatment that does not treat the subject with any GnRH angonist and antagonist.

In some embodiments, the present disclosure provides a method of treating prostate cancer (e.g., any of those described herein) in a subject in need thereof, with a combination therapy, which comprises administering to the subject a therapeutically effective amount of the pharmaceutical composition herein (e.g., any of those described herein, such as any of [1]-[37] of the Summary section herein), and one or more additional therapies. The one or more additional therapies can be administered to the subject concurrently or sequentially in any order with administering the pharmaceutical composition herein, which can be via the same or different route of administration. In some embodiments, the method herein comprises treating the subject with a radiotherapy or surgery. In some embodiments, the method comprises administering to the subject one or more other agents selected from anticancer agents, hormone ablation agents, anti-androgen agents, differentiating agents, anti-neoplastic agents, kinase inhibitors, anti-metabolite agents, alkylating agents, antibiotic agents, immunological agents, interferon-type agents, intercalating agents, growth factor inhibitors, cell cycle inhibitors, enzymes, topoisomerase inhibitors, biological response modifiers, mitotic inhibitors, matrix metalloprotease inhibitors, genetic therapeutics, or combinations thereof. In some embodiments, the method comprises administering to the subject one or more other agents selected from a chemotherapeutic drug, hormone replacement drug, or hormone ablation drug. In some embodiments, the method comprises treating the subject with an androgen deprivation therapy. While many of the combination therapies below are described as in connection with various treatments for prostate cancer, the present disclosure is not so limited. And in some embodiments, the combination therapies described below can also be used in the treatment of other diseases or disorders described herein, such as other cancers described herein.

In more particular embodiments, the combination therapy typically includes administering to the subject a glucocorticoid. For example, in some embodiments, the method comprises administering to the subject one or more agents selected from hydrocortisone, prednisone, prednisolone, methylprednisolone, and dexamethasone. However, in some embodiments, a glucococorticoid replacement therapy (e.g., administering a glucocorticoid, such as hydrocortisone, prednisone, prednisolone, methylprednisolone, or dexamethasone) is not desired. For example, a glucocorticoid may be contraindicated for the subject, who may have an underlying condition, such as diabetics. In some embodiments, the method can also be characterized in that the subject is not treated with a glucocorticoid replacement therapy. In some embodiments, the subject is not treated with an agent selected from hydrocortisone, prednisone, prednisolone, methylprednisolone, and dexamethasone. In some embodiments, the method can comprise administering to the subject a mineralocorticoid receptor antagonist, such as eplerenone. For example, in any of the embodiments herein when glucococorticoid replacement therapy is not desired and/or not administered, the method can can comprise administering to the subject a mineralocorticoid receptor antagonist, such as eplerenone.

The combination therapy for the methods herein can also include an androgen deprivation therapy, such as through administering to the subject a gonadotropin-releasing hormone (GnRH) analog. When included, suitable GnRH analogs for the combination therapy are not particularly limited and include both GnRH agonists and GnRH antagonists. For example, in some embodiments, the method can comprise administering to the subject a gonadotropin-releasing hormone (GnRH) agonist, such as buserelin, leuprolide, deslorelin, fertirelin, histrelin, gonadorelin, lecirelin, goserelin, nafarelin, peforelin or triptorelin, and/or a GnRH antagonist, such as abarelix, cetrorelix, degarelix, ganirelix, elagolix, linzagolixa, or relugolix. In some embodiments, the subject is not administered any of the GnRH agonists and GnRH antagonists described herein.

### Inhibition of Androgen Receptor Activities

In some embodiments, the combination therapy includes treating the subject to reduce androgen receptor (AR) activities, such as an AR antagonist or an agent otherwise downregulating or inhibiting AR activities.

In some embodiments, the method can include administering to the subject an androgen receptor (AR) antagonist. Various AR antagonists are known in the art, which include without limitation 1^{st} and 2^{nd}-generations AR antagonists, see e.g., Rice, M. A., et al. Front Oncol. 9:801 (2019), and third-generation AR antagonists, such as an N-terminal domain inhibitor. In some embodiments, the method comprises administering to the subject a 1^{st}-generation androgen receptor antagonist, which includes without limitation, proxalutamide, bicalutamide, flutamide, nilutamide, topilutamide, etc. In some embodiments, the method comprises administering to the subject a 2^{nd}-generation androgen receptor antagonist, which includes without limitation, for example, apalutamide, darolutamide or enzalutamide. In some embodiments, the method comprises administering to the subject apalutamide. In some embodiments, the method comprises administering to the subject enzalutamide. In some embodiments, the method comprises administering to the subject a 3^{rd}-generation androgen receptor antagonist, such as an N-terminal domain inhibitor. N-terminal domain inhibitors are known in the art. Non-limiting useful examples include any of those described in U.S. Application Publication No. 2020/0123117, the content of which is herein incorporated by reference. It should be noted that in embodiments where an AR antagonist is administered, one or more such antagonists can be administered, which can be selected from 1^{st}, 2^{nd} or 3^{rd} AR antagonists alone, or in any combination.

In addition to agents directly targeting androgen receptor, other methods and/or agents that modulate androgen receptor activities, including for example, modulation of upstream kinase activities and/or androgen receptor transcriptional activities, can also be used in the combination therapy herein. For example, in some embodiments, the combination therapy can include administering to the subject one or more upstream kinase modulators, the activation or inhibition of which can reduce AR activities. Such upstream kinases are known in the art, for example, as described in Shah, K. and Bradbury, N. A., Cancer cell microenviron. 2(4):doi:10.14800/ccm.1023 (2015), and Koul H. K. et al. Genes & Cancer 4(9-10):342-359 (2013). In some embodiments, the method comprises administering to the subject one or more kinase modulators selected from FLT-3 (FMS-like tyrosine kinase) inhibitors, AXL (anexelekto) inhibitors (e.g., Gilteritinib), CDK (cyclin dependent kinase) inhibitors, such as CDK1, 2, 4, 5, 6, 7, or 9 inhibitors, retinoblastoma (Rb) inhibitors, protein kinase B (AKT) inhibitors, SRC inhibitors, IkappaB kinase 1 (IKK1) inhibitors, PIM-1 modulators, Lemur tyrosine kinase 2 (LMTK2) modulators, Lyn inhibitors, Aurora A inhibitors, ANPK (a nuclear protein kinase) inhibitors, extracellular-signal regulated kinase (ERK) modulators, c-jun N-terminal kinase (JNK) modulators, Big MAP kinase (BMK) modulators, p38 mitogen-activated protein kinases (MAPK) modulators, and combinations thereof. Suitable kinase modulators/inhibitors are not particularly limited, which include any of those known, for example, small molecule drugs, polypeptides including antibodies such as monoclonal antibodies or antigen binding fragments thereof, RNA or DNA based agents.

In some embodiments, the combination therapy can include administering to the subject an agent that downregulates AR or otherwise inhibits AR activities. Without wishing to be bound by theories, AR activities can be affected on the genomic and/or the transcription level of AR itself, or the genomic and/or the transcription level of those upstream targets of AR that play a role in regulating AR activities and those downstream targets that are regulated by AR, using a variety of molecules which interfere with transcription and/or translation (e.g., RNA silencing agents (e.g., antisense, siRNA, shRNA, micro-RNA), Ribozyme and DNAzyme), or on the protein level using e.g., antagonists, enzymes that cleave the polypeptide, small molecules that interfere with the protein's activity (e.g., competitive ligands) and the like.

In some embodiments, downregulation of AR or inhibition of AR activities can be achieved through RNA silencing of a target gene (e.g., AR or suitable upstream and downstream targets of AR as described herein, etc.). As used herein, the phrase “RNA silencing” refers to a group of regulatory mechanisms (e.g., RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In some embodiments, the RNA silencing agent is capable of preventing complete processing (e.g., the full translation and/or expression) of an mRNA molecule through a post-transcriptional silencing mechanism. RNA silencing agents include noncoding RNA molecules, for example, RNA duplexes comprising paired strands, as well as precursor RNAs from which such small non-coding RNAs can be generated. Exemplary RNA silencing agents include double-stranded RNAs (dsRNAs) such as short interfering RNAs (siRNAs), miRNAs and shRNAs. In one embodiment, the RNA silencing agent is capable of inducing RNA interference. In another embodiment, the RNA silencing agent is capable of mediating translational repression. The strands of a double-stranded interfering RNA (e.g., an siRNA) may be connected to form a hairpin or stem-loop structure (e.g., an shRNA or sh-RNA). Thus, as mentioned, the RNA silencing agent of some embodiments of the disclosure may also be a short hairpin RNA (shRNA).

It will be appreciated that the RNA silencing agent of some embodiments of the present disclosure need not be limited to those molecules containing only RNA, but further encompasses chemically modified nucleotides and non-nucleotides.

In some embodiments, the RNA silencing agent provided herein can be functionally associated with a cell-penetrating peptide. As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell.

According to another embodiment, the RNA silencing agent may be a miRNA or a mimic thereof. The term “microRNA”, “miRNA”, and “miR” are synonymous and refer to a collection of non-coding single-stranded RNA molecules of about 19-28 nucleotides in length, which regulate gene expression. miRNAs are found in a wide range of organisms and have been shown to play a role in development, homeostasis, and disease etiology. The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs).

Downregulation of AR or inhibition of AR activities can also be achieved by gene editing of a target gene (e.g., AR or suitable upstream and downstream targets of AR as described herein, etc.). Gene editing can be performed, for example, with a clustered regularly interspaced short palindromic repeats CRISPR-CAS9 system. CRISPR-CAS9 systems have been described in the literature and can include, for example, CAS9 and a guide RNA. Other gene editing techniques have also been described in the literature and can also be used.

Another agent capable of downregulating a target (e.g., AR or suitable upstream and downstream targets of AR as described herein, etc.) is a DNAzyme molecule capable of specifically cleaving an mRNA transcript or DNA sequence of the target. DNAzymes are single-stranded polynucleotides which are capable of cleaving both single and double stranded target sequences. (Breaker et al., Chemistry and Biology 1995; 2:655; Santoro et al., Proc. Natl. Acad. Sci. USA 1997; 943:4262.) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions. (Santoro et al., Khachigian, Curr. Opin. Mol. Ther. 2002; 4:119-121.)

Downregulation of a target (e.g., AR or suitable upstream and downstream targets of AR as described herein, etc.) can also be affected by using an antisense polynucleotide capable of specifically hybridizing with an mRNA transcript encoding the target.

Another agent capable of downregulating a target (e.g., AR or suitable upstream and downstream targets of AR as described herein, etc.) is a ribozyme molecule capable of specifically cleaving an mRNA transcript encoding a target. Ribozymes are being increasingly used for the sequence-specific inhibition of gene expression by the cleavage of mRNAs encoding proteins of interest. (Welch et al., Curr. Opin. Biotechnol. 1998; 9:486-96.)

Another agent capable of downregulating a target (e.g., AR or suitable upstream and downstream targets of AR as described herein, etc.) is any molecule which binds to and/or cleaves the target. Such molecules can be antagonists of the target, or inhibitory peptides of the target.

Another agent which can be used along with some embodiments of the present disclosure to downregulate a target (e.g., AR or suitable upstream and downstream targets of AR as described herein, etc.) is a molecule which prevents target activation and/or substrate binding.

Another agent which can be used along with some embodiments of the present disclosure to downregulate AR or inhibit AR's activities is an androgen receptor degrader, such as those based on PROteolysis TArgeting Chimeric (PROTAC) technology. See, e.g., Kregel, S. et al. Neoplasia 22(2):111-119 (2020).

Another agent which can be used along with some embodiments of the present disclosure to downregulate a target (e.g., AR or suitable upstream and downstream targets of AR as described herein, etc.) is to repress or downregulate the activation of the target's transcriptional activity, more particularly, AR's transcriptional activities. For example, such agent can interfere with the nuclear translocation of AR, downregulate the protein level of AR, decrease hormone binding to AR, interfere with recruitment of transcriptional cofactors (e.g., steroid receptor coactivator 1 (SRC1) and transcriptional intermediary factor 2 (TIF2)), interefer with with AR-DNA-binding, e.g., the binding to specific DNA response elements (AREs or, androgen response elements), inhibit AR recruitment to an AR target gene enhancer, and/or inhibit AR-chromatin binding etc. or otherwise inhibit the DNA-binding-dependent or non-DNA-binding-dependent AR signaling pathways. Suitable agents that can inhibit or interfere with AR transcriptional activities include any of those known in the art and any of those agents exemplified herein that are capable of inhibiting or interfering with such activities. For example, certain AR antagonists such as the 1^{st} generation AR antagonists (e.g., bicalutamide) are known to inhibit AR transcriptional activities by inhibiting nuclear translocation of AR. Other agents, such as arsenic compounds (e.g., arsenic trioxide), were also known to inhibit AR transcriptional activity. See e.g., Rosenblatt A. E., et al, Mol. Endocrinol. 23(3):412-421 (2009).

In some embodiments, the combination therapy can include administering to the subject one or more chemotherapeutic agents. Suitable chemotherapeutic agents include any of those known in the art. In some embodiments, the method comprises administering to the subject a taxane based chemotherapeutic agent (e.g., docetaxel, cabazitaxel, paclitaxel, etc.) and/or platinum based chemotherapeutic agent (e.g., cisplatin, carboplatin, oxaliplatin, etc.).

In some embodiments, the combination therapy can include treating the subject with a radiotherapy. Suitable radiotherapy includes any of those known in the art. In some embodiments, the method comprises treating the subject with stereotactic body radiotherapy or neutron radiation.

In some embodiments, the combination therapy can include treating the subject with Radium-223, e.g., Xofigo (Radium-223 dichloride) injection.

In some embodiments, the combination therapy can include administering to the subject one or more immunotherapies. Suitable immunotherapies include any of those known in the art. In some embodiments, the method comprises administering to the subject Sipuleucel-T. In some embodiments, the method comprises administering to the subject an immune checkpoint inhibitor. For example, in some embodiments, the method comprises administering to the subject an anti-PD-1 antibody, such as pembrolizumab or nivolumab, and/or an anti-PD-L1 antibody, such as avelumab or atezolizumab. In some embodiments, the method comprises administering to the subject an anti-CTLA-4 antibody, such as ipilimumab.

In some embodiments, the combination therapy can include administering to the subject a bispecific T-cell engager (BiTE) therapy, such as blinatumomab or solitomab.

In some embodiments, the combination therapy can include administering to the subject one or more poly ADP ribose polymerase (PARP) inhibitors. In some embodiments, the subject having prostate cancer also has DNA repair defects. In some embodiments, the subject having prostate cancer does not have DNA repair defects. Suitable PARP inhibitors include any of those known in the art. For example, in some embodiments, the method comprises administering to the subject a PARP inhibitor selected from niraparib, rucaparib, olaparib, talazoparib, veliparib, and fluzoparib.

In some embodiments, the combination therapy can include administering to the subject one or more kinase inhibitors. In some embodiments, the subject is characterized as having an abnormal level of the respective kinase. In some embodiments, the kinase inhibitor can reduce the activity of androgen receptor or otherwise beneficial to cancer treatment. Suitable kinase inhibitors include any of those known in the art. For example, in some embodiments, the method comprises administering to the subject a kinase inhibitor selected from sunitinib, dasatinib, cabozantinib, erdafitinib, dovitinib, capivasertib, onvansertib, ipatasertib, afuresertib, alisertib, apitolisib, and opaganib.

In some embodiments, the combination therapy can include administering to the subject one or more bone protecting agents. In such embodiments, typically, the subject is characterized as having prostate cancer (e.g., CRPC) with bone metastasis. Suitable bone protecting agents include any of those known in the art. For example, in some embodiments, the method comprises administering to the subject a bone protecting agent selected from denosumab and zolendronic acid.

In some embodiments, the combination therapy can include administering to the subject one or more additional agents that are useful for treating prostate cancer, by itself or in combination with an abiraterone medication such as the abiraterone prodrugs herein. Such additional agents are not particularly limited. For example, in some embodiments, the method comprises administering to the subject a therapeutic agent selected from 1) an anti-IL23 targeting monoclonal antibody, e.g., tildrakizumab; 2) a selenium, such as sodium selenite; 3) an EZH2 inhibitor, e.g., CPI-1205, GSK2816126, or tazemetostat; 4) a CDK4/6 inhibitor, e.g., palbociclib, ribociclib, abemaciclib; 6) a bromodomain and extra-terminal domain (BET) inhibitor, e.g., CCS1477, INCB057643, alobresib, ZEN-3694, or molibresib (GSK525762); 7) an anti-CD105 antibody, e.g., TRC105 or carotuximab; 8) niclosamide; 9) an A2A receptor antagonist, e.g., AZD4635; 10) a phosphoinositide 3-kinase (PI3K) inhibitor, e.g., AZD-8186, buparlisib, or dactolisib; 11) a further non-steroidal CYP17A1 inhibitor, e.g. seviteronel; 12) an antiprogestogen, e.g., onapristone; 13) navitoclax; 14) an HSP90 inhibitor, e.g., onalespib (AT13387); 15) an HSP27 inhibitor, e.g., OGX-427; 16) a 5-alpha-reductase inhibitor, e.g., dutasteride; 17) metformin; 18) AMG-386; 19) dextromethorphan; 20) theophylline; 21) hydroxychloroquine; and 22) lenalidomide. In some embodiments, the combination therapy can include administering to the subject one or more one or more kinase modulators selected from FLT-3 (FMS-like tyrosine kinase) inhibitors, AXL (anexelekto) inhibitors (e.g., Gilteritinib), CDK (cyclin dependent kinase) inhibitors, such as CDK1, 2, 4, 5, 6, 7, or 9 inhibitors, retinoblastoma (Rb) inhibitors, protein kinase B (AKT) inhibitors, SRC inhibitors, IkappaB kinase 1 (IKK1) inhibitors, PIM-1 modulators, Lemur tyrosine kinase 2 (LMTK2) modulators, Lyn inhibitors, Aurora A inhibitors, ANPK (a nuclear protein kinase) inhibitors, extracellular-signal regulated kinase (ERK) modulators, c-jun N-terminal kinase (JNK) modulators, Big MAP kinase (BMK) modulators, p38 mitogen-activated protein kinases (MAPK) modulators, and combinations thereof. In some embodiments, a cell therapy, such as a T cell mediated cell therapy including central memory T cells, can also be part of the combination therapy.

In some embodiments, the combination therapy can include administering to the subject one or more agents selected from 1) a poly (ADP-ribose) polymerase (PARP) inhibitor including but not limited to olaparib, niraparib, rucaparib, talazoparib; 2) an androgen receptor ligand binding domain inhibitor including but not limited to enzalutamide, apalutamide, darolutamide, bicalutamide, nilutamide, flutamide, ODM-204, TAS3681; 3) an additional inhibitor of CYP17 including but not limited to galeterone, abiraterone, abiraterone acetate; 4) a microtubule inhibitor including but not limited to docetaxel, paclitaxel, cabazitaxel (XRP-6258); 5) a modulator of PD-1 or PD-L1 including but not limited to pembrolizumab, durvalumab, nivolumab, atezolizumab; 6) a gonadotropin releasing hormone agonist including but not limited to cyproterone acetate, leuprolide; 7) a 5-alpha reductase inhibitor including but not limited to finasteride, dutasteride, turosteride, bexlosteride, izonsteride, FCE 28260, SKF105,111; 8) a vascular endothelial growth factor inhibitor including but not limited to bevacizumab (Avastin); 9) a histone deacetylase inhibitor including but not limited to OSU-HDAC42; 10) an integrin alpha-v-beta-3 inhibitor including but not limited to VITAXIN; 11) a receptor tyrosine kinase inhibitor including but not limited to sunitumib; 12) a phosphoinositide 3-kinase inhibitor including but not limited to alpelisib, buparlisib, idealisib; 13) an anaplastic lymphoma kinase (ALK) inhibitor including but not limited to crizotinib, alectinib; 14) an endothelin receptor A antagonist including but not limited to ZD-4054; 15) an anti-CTLA4 inhibitor including but not limited to MDX-010 (ipilimumab); 16) an heat shock protein 27 (HSP27) inhibitor including but not limited to OGX 427; 17) an androgen receptor degrader including but not limited to ARV-330, ARV-110; 18) an androgen receptor DNA-binding domain inhibitor including but not limited to VPC-14449; 19) a bromodomain and extra-terminal motif (BET) inhibitor including but not limited to BI-894999, GSK525762, GS-5829; 20) an androgen receptor N-terminal domain inhibitor including but not limited to a sintokamide; 21) an alpha-particle emitting radioactive therapeutic agent including but not limited to radium 233 or a salt thereof; 22) niclosamide; or related compounds thereof; 23) a selective estrogen receptor modulator (SERM) including but not limited to tamoxifen, raloxifene, toremifene, arzoxifene, bazedoxifene, pipindoxifene, lasofoxifene, enclomiphene; 24) a selective estrogen receptor degrader (SERD) including but not limited to fulvestrant, ZB716, OP-1074, elacestrant, AZD9496, GDC0810, GDC0927, GW5638, GW7604; 25) an aromitase inhibitor including but not limited to anastrazole, exemestane, letrozole; 26) selective progesterone receptor modulators (SPRM) including but not limited to mifepristone, lonaprison, onapristone, asoprisnil, lonaprisnil, ulipristal, telapristone; 27) a glucocorticoid receptor inhibitor including but not limited to mifepristone, COR108297, COR125281, ORIC-101, PT150; 28) CDK4/6 inhibitors including palbociclib, abemaciclib, ribociclib; 29) HER2 receptor antagonist including but not limited to trastuzumab, neratinib; and 30) a mammalian target of rapamycin (mTOR) inhibitor including but not limited to everolimus, temsirolimus.

The combination therapy herein is not particularly limited to any specific numbers of additional therapies. For example, in addition to administering the pharmaceutical composition herein and an optional glucocorticoid such as hydrocortisone, prednisone, prednisolone, methylprednisolone, and dexamethasone, the combination therapy typically can include additional 1, 2, 3, 4, 5, 6, or more therapies described herein. For example, in some embodiments, the combination therapy can include one additional therapy, e.g., any one of those described herein, for example, a GnRH agonist, a GnRH antagonist, an androgen receptor antagonist, a chemotherapy, a PARP inhibitor, a kinase inhibitor, an immunotherapy, a radiation therapy, surgery, an androgen deprivation therapy, etc. In some embodiments, the combination therapy can include two or more additional therapies described herein. For example, in some particular embodiments, the combination therapy can include administering to the subject a PARP inhibitor and an androgen deprivation therapy. In some embodiments, the combination therapy can include administering to the subject a GnRH agonist and a radiation therapy. In some embodiments, the combination therapy can include administering to the subject a GnRH agonist, a chemotherapeutic agent, and a radiation therapy. In some embodiments, the combination therapy can include administering to the subject an androgen receptor antagonist (e.g., 1^{st}, 2^{nd} and/or 3^{rd} generation AR antagonist), a GnRH agonist, and optionally a radiation therapy, a chemotherapeutic agent, indomethacin, or 5-alpha reductase inhibitor. In some embodiments, the combination therapy can include administering to the subject an androgen receptor antagonist (e.g., 1^{st}, 2^{nd}, and/or 3^{rd} generation AR antagonist) and a radiation therapy. In some embodiments, the combination therapy can include administering to the subject an androgen receptor antagonist (e.g., 1^{st}, 2^{nd} and/or 3^{rd} generation AR antagonist) and a chemotherapeutic agent. In some embodiments, the combination therapy can include administering to the subject an androgen receptor antagonist (e.g., 1^{st}, 2^{nd} and/or 3^{rd} generation AR antagonist) and an anti-CTLA4 antibody. It should be understood that these combinations discussed are examples of useful combinations, which are in no way limiting, and other combinations of the additional therapies described herein are allowed.

It should be noted that in some embodiments, the method of treating prostate cancer (e.g., any of those described herein) herein is not in conjunction with a combination therapy. For example, the method comprises administering to the subject a therapeutically effective amount of the pharmaceutical composition herein, without the one or more additional therapies described herein.

The pharmaceutical composition herein can be administered to a subject in need thereof as the only source of abiraterone. However, in some embodiments, other abiraterone medications/formulations are not excluded. For example, in some embodiments, the administering herein can be combined, either concurrently or sequentially in any order, with an oral administration of abiraterone acetate, such as the Zytiga® formulation. In some embodiments, the subject can use the pharmaceutical composition herein as a supplement to an existing abiraterone therapy.

Provided herein are formulations, methods, and kits for treating a subject with a sex hormone-dependent benign or malignant disorder such as prostate cancer. Also provided are methods for preparing the formulations useful for treating a subject with a sex hormone-dependent benign or malignant disorder (such as prostate cancer), an androgen receptor driven cancer, a syndrome due to androgen excess, and/or a syndrome due to glucocorticoid excess such as hypercortisolemia. Reference will now be made in detail to representative embodiments, examples of which are illustrated in the accompanying drawings.

The term “subject” as used herein means, but is not limited to, an animal or human in need of or capable of receiving chemotherapy for a sex hormone-dependent benign or malignant disorder such as, for example, an androgen-dependent disorder or an estrogen-dependent disorder (including prostate cancer and breast cancer), an androgen receptor driven cancer, an animal or human in need of or capable of receiving therapy for non-oncologic syndromes due to androgen excess, such as endometriosis, polycystic ovary syndrome, congenital adrenal hyperplasia (e.g., classical or nonclassical congenital adrenal hyperplasia), precocious puberty, hirsutism, etc., and/or due to glucocorticoid excess such as hypercortisolemia, such as Cushing's syndrome or Cushing's disease. In preferred embodiments, the subject is a human subject.

The term “other drug or agent” as used herein (when, for example, referring to prior, simultaneous, and post-administration of at least one other drug or agent with at least one abiraterone prodrug formulation) means at least one other compound, formulation, molecule, biologic, or the like, capable of enhancing the efficacy of the formulation(s), decreasing an undesirable side effect(s) of the formulation(s), or improving the treatment of the particular disorder. Any suitable routes of administration of such “other drug or agent” can be used, for example, oral administration, parenteral administration, etc. A person skilled in the art of treating a subject having a sex hormone-dependent benign or malignant disorder (such as an androgen-dependent disorder or an estrogen-dependent disorder), an androgen receptor driven cancer, syndromes due to androgen excess syndrome, and/or syndromes due to glucocorticoid excess such as hypercortisolemia would know and understand how to choose and use such “other drug or agent” for the intended purpose(s).

The formulations can optionally be administered via a modified-release device or method. The term “modified-release” as used herein should be understood as encompassing delayed release, prolonged or extended release, sustained release, or a targeted release, etc. For example, in some embodiments, the modified release device or method can further prolong the release of abiraterone of the prodrugs and formulations of the present disclosure. In some embodiments, the modified release device or method can also include any device or method capable of releasing an agent or product (for example, a drug or a biologic) at a time later than immediately following its administration (and can include, for example, implants). Various modified release devices have been described (Stubbe et al., Pharm. Res. 21:1732, 2004) and could be applicable to the representative embodiments. Modified-release devices and methods can be identified and employed without undue experimentation by a person skilled in the art after consideration of all criteria and use of best judgment on the subject's behalf.

The formulations and agents of the embodiments are administered in a pharmacologically or physiologically acceptable and effective amount to reduce or eliminate the presence, for example, of prostate tumor tissue and abnormal or malignant prostate cells in a subject presenting with prostate cancer. Similarly, the formulations and agents of the embodiments are administered alone or in combination with other therapeutic agents or therapeutic modalities (for example, radiotherapy and surgery) in prophylactically or therapeutically effective amounts, which are to be understood as amounts meeting the intended prophylactic or therapeutic objectives and providing the benefits available from administration of such formulations and agents.

The terms “effective amount,” “effective dose,” and “therapeutic blood plasma concentration” as used herein mean, but are not limited to, an amount, dose, or concentration capable of treating, delaying, slowing, inhibiting, or eliminating the onset, existence or progression of a disorder, disease or condition. For example, an “effective amount,” “effective dose,” or “therapeutic blood plasma concentration” is capable of reducing or eliminating the presence of prostate tumor tissue and abnormal or malignant prostate cells in a subject presenting with prostate cancer, which is sufficient to cure (partly or completely) illness or prevent the onset or further spread of disorder, disease or condition. For further example, an effective amount of formulation refers to the amount administered alone or in combination with other therapeutic agents or therapeutic modalities (for example, radiotherapy and surgery) to achieve clinically significant reduction in tumor burden. A person skilled in the art would understand when a clinically significant reduction in tumor burden (or improvement of a sex hormone-dependent benign or malignant disorder or another disorder or syndrome described herein) has occurred following administration of a formulation. An “effective amount,” “effective dose,” or “therapeutic blood plasma concentration” is understood to be an amount, dose, or concentration not critically harmful to the subject and, in any case, where any harmful side effects are outweighed by benefits. By way of example only, an effective amount or dose of an abiraterone decanoate formulation means an amount capable of attaining blood plasma concentrations of at least 1 ng/ml, e.g., at least 1 ng/ml, at least 2 ng/ml, at least 4 ng/ml, or at least 8 ng/ml, of abiraterone in the subject following oral administration of the pharmaceutical composition herein, and the efficacious blood plasma concentrations are attained for 12 hours or more, preferably, 24 hours or more following administration.

In general, the dosage ranges for administration of the formulation according to the present disclosure are those that produce the desired effect(s). The useful dosage to be administered will vary depending on the age, weight, and health of the subject treated, the mode, route, and schedule of administration, the response of the individual subject, and the type or staging of prostate cancer (or severity of a sex hormone-dependent benign or malignant disorder or another syndrome or disorder described herein) against which treatment with the formulation is sought. The dosage will also vary with the nature or the severity of the primary tumor and other underlying conditions, with epidemiologic conditions, with the concomitant use of other active compounds, and the route of administration. In addition, the dosage will be determined by the existence of any adverse side effects such as local hypersensitivity, systemic adverse effects, and immune tolerance.

An effective dose of the formulations (and other agent(s)) can be determined without undue experimentation (for example, by pharmacokinetic studies) by a person skilled in the art after consideration of all criteria and use of best judgment on the patient's behalf (and will most often be contingent upon the particular formulation utilized). The dosage to be administered will depend upon the particular case, but in any event, it is the amount sufficient to induce clinical benefit against, or improvement of, a sex hormone-dependent benign or malignant disorder (such as prostate cancer), an androgen receptor driven cancer, a syndrome due to androgen excess, and/or a syndrome due to glucocorticoid excess such as hypercortisolemia.

The formulations and agents of the embodiments can, optionally, be administered in combination with (or can include) one or more pharmaceutically acceptable carriers, diluents, or excipients. Formulations, administration techniques, pharmaceutical compositions, methods of preparing pharmaceutical compositions, and pharmaceutically acceptable carriers, diluents, and excipients are known in the art and are described, for example, in “Remington: The Science and Practice of Pharmacy” (formerly “Remington's Pharmaceutical Sciences,” University of the Sciences in Philadelphia, Lippincott, Williams & Wilkins, Philadelphia, Pa. (2005)), the disclosure of which is hereby incorporated by reference.

The abbreviations used herein have their conventional meaning within the chemical and biological arts.

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

As used herein, the term “about” modifying an amount related to the disclosure refers to variation in the numerical quantity that can occur, for example, through routine testing and handling; through error in such testing and handling; through differences in the manufacture, source, or purity of ingredients/materials employed in the disclosure; and the like. As used herein, “about” a specific value also includes the specific value, for example, about 10% includes 10%. Whether or not modified by the term “about”, the claims include equivalents of the recited quantities. In one embodiment, the term “about” means within 25% of the reported numerical value.

It is also meant to be understood that a specific embodiment of a variable moiety herein may be the same or different as another specific embodiment having the same identifier.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

As used herein, the term “alkyl” as used by itself or as part of another group refers to a straight- or branched-chain saturated aliphatic hydrocarbon. In some embodiments, the alkyl can include one to thirty carbon atoms (i.e., C₁₋₃₀ alkyl or alternatively expressed as C₁-C₃₀ alkyl) or the number of carbon atoms designated (i.e., a C₁ alkyl such as methyl, a C₂ alkyl such as ethyl, a C₃ alkyl such as propyl or isopropyl, etc.). In one embodiment, the alkyl group is a straight chain C₁₋₁₆ alkyl group. In another embodiment, the alkyl group is a branched chain C₃₋₁₆ alkyl group. To be clear, when a range of carbon numbers is listed, it encompasses each individual integer within the range and sub-ranges between such integers as would be understood by those skilled in the art. For example, “C₇₋₁₆” herein encompasses, C₇, C₈, C₉, C₁₀, C₁₁, C₁₂, C₁₃, C₁₄, C₁₅, C₁₆, C₇₋₁₆, C₇₋₁₅, C₇₋₁₄, C₇₋₁₃, C₇₋₁₂, C₇₋₁₁, C₇₋₁₀, C₇₋₉, C₇₋₈, C₈₋₁₆, C₈₋₁₅, C₈₋₁₄, C₈₋₁₃, C₈₋₁₂, C₈₋₁₁, C₈₋₁₀, C₈₋₉, C₉₋₁₆, C₉₋₁₅, C₉₋₁₄, C₉₋₁₃, C₉₋₁₂, C₉₋₁₁, C₉₋₁₀, C₁₀₋₁₆, C₁₀₋₁₅, C₁₀₋₁₄, C₁₀₋₁₃, C₁₀₋₁₂, C₁₀₋₁₁, C₁₁₋₁₆, C₁₁₋₁₅, C₁₁₋₁₄, C₁₁₋₁₃, C₁₁₋₁₂, C₁₂₋₁₆, C₁₂₋₁₅, C₁₂₋₁₄, C₁₂₋₁₃, C₁₃₋₁₆, C₁₃₋₁₅, C₁₃₋₁₄, C₁₄₋₁₆, C₁₄₋₁₅, and C₁₅₋₁₆. Other ranges as described herein such as “number of carbons between 5 and 16” etc. should be understood similarly.

As used herein, the term “cycloalkyl” as used by itself or as part of another group refers to saturated and partially unsaturated (e.g., containing one or two double bonds) cyclic aliphatic hydrocarbons containing one to three rings having from three to twelve carbon atoms (i.e., C₃₋₁₂ cycloalkyl) or the number of carbons designated. In one embodiment, the cycloalkyl group has two rings. In one embodiment, the cycloalkyl group has one ring. In another embodiment, the cycloalkyl group is a C3-s cycloalkyl group. In another embodiment, the cycloalkyl group is a C₃₋₆ cycloalkyl group. “Cycloalkyl” also includes ring systems wherein the cycloalkyl ring, as defined above, is fused with one or more aryl or heteroaryl groups wherein the point of attachment is on the cycloalkyl ring, and in such instances, the number of carbons continue to designate the number of carbons in the cycloalkyl ring system. Non-limiting exemplary cycloalkyl groups include cyclopropyl, cyclobutyl, cyclopentyl, cyclohexyl, cycloheptyl, cyclooctyl, norbornyl, decalin, adamantyl, cyclopentenyl, and cyclohexenyl.

As used herein, the term “alkenyl” as used by itself or as part of another group refers to a straight- or branched-chain aliphatic hydrocarbon containing one or more (e.g., 1, 2, or 3) carbon-to-carbon double bonds. In one embodiment, the alkenyl group is a C₂₋₁₆ alkenyl group.

As used herein, the term “alkynyl” as used by itself or as part of another group refers to a straight- or branched-chain aliphatic hydrocarbon containing one or more (e.g., 1, 2, or 3) carbon-to-carbon triple bonds. In one embodiment, the alkynyl has one carbon-carbon triple bond. In one embodiment, the alkynyl group is a C₂₋₁₆ alkynyl group.

As used herein, the term “abiraterone prodrug(s)” includes any of the compounds described herein according to Formula I or II, a lipophilic ester of abiraterone, isotopically labeled compound(s) thereof (e.g., deuterium enriched compounds), possible stereoisomers thereof (including diastereoisomers, enantiomers, and racemic mixtures), tautomers thereof, conformational isomers thereof, and/or pharmaceutically acceptable salts thereof (e.g., acid addition salt such as HCl salt). Hydrates and solvates of the prodrugs are considered compositions of the present disclosure, wherein the prodrug(s) is in association with water or solvent, respectively. Some of the prodrugs can also exist in various polymorphic forms or amorphous forms. The abiraterone prodrugs described herein also include those compounds that readily undergo chemical changes under physiological conditions to provide active abiraterone. Additionally, prodrugs can be converted by chemical or biochemical methods in an ex vivo environment. In any of the embodiments described herein, unless otherwise specified or contrary from context, the abiraterone prodrug can be abiraterone decanoate.

The abiraterone prodrugs described herein can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, oxygen, and nitrogen, include, but are not limited to ²H, ³H, ³C, ¹⁴C, ¹⁵N, and ¹⁸O. Compounds that contain other isotopes of these and/or other atoms are within the scope of this disclosure.

Solid and dashed wedge bonds indicate stereochemistry as customary in the art.

The following examples are provided for illustration purposes only and are in no way intended to limit the scope of the claimed subject matter.

### Example 1A. Large Scale Preparation of Abiraterone Decanoate from Decanoic Acid

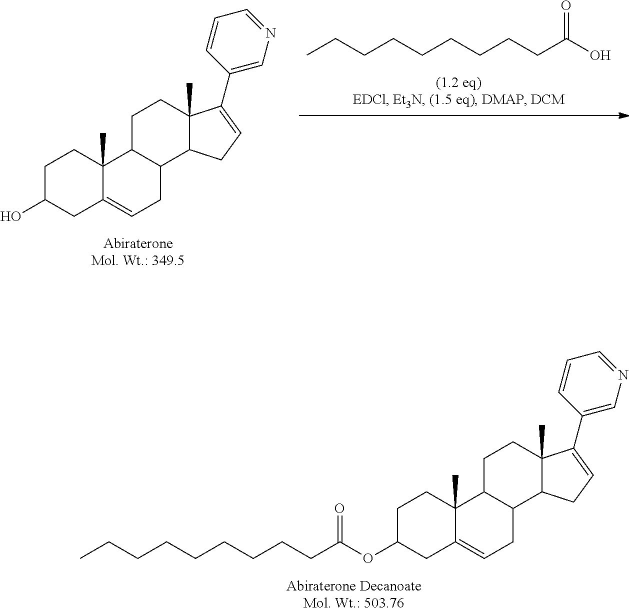

To a suspension of Abiraterone (381.9 g, 1.09 mol) in dichloromethane (3500 mL) was added triethylamine (165 g, 1.64 mol) and a catalytic amount of DMAP (13.35 g, 0.109 mol). Decanoic acid (225 g, 1.31 mol) as a solution in dichloromethane (500 mL) was added to the suspension, followed by EDCI (293 g, 1.53 mol) and the reaction then agitated for 19 h at 20-25° C.

10 wt % aq NaH₂PO₄ (4000 mL) was then added and the reaction was agitated for 20 min. The organic layer was separated and extracted with 10 wt % aq NaH₂PO₄ (2000 mL) and brine (2000 mL). The organic layer was solvent exchanged with acetonitrile (4750 mL) and concentrated to 3100 g keeping temperature of bath <40° C. The suspension was diluted with acetonitrile (900 g). The solids were isolated by filtration to afford 510 g of crude abiraterone decanoate.

510 g of the crude abiraterone decanoate was dissolved in acetone (4000 mL) at 40° C. The solution was filtered through a filter paper. The filtrate was transferred to a 12 L 3-neck flask, diluted to 5100 g and reheated to 40° C. to form a solution. The solution was cooled slowly to 20° C. to form a suspension. This was diluted with water (1020 mL) and agitated at RT overnight. The solid was filtered and the flask was rinsed with the filtrate and transferred to filter funnel. The wet cake was transferred to drying tray and dried at 40-45° C. in vacuum oven overnight to obtain 457.1 g (90% yield) as white solid, the crystalline form of this solid is designated as Form A. ¹H NMR (CDCl₃, 400 MHz): d_{H} 8.62 (d, 1H, J=1.9 Hz), 8.31 (dd, 1H, J=4.9, 1.6 Hz), 7.64 (dt, 1H, J=7.9, 1.9 Hz), 7.21 (ddd, 1H, J=8.0, 4.9, 0.8 Hz), 6.01-5.97 (m, 1H), 5.44-5.40 (m, 1H), 4.68-4.58 (m, 1H), 2.39-2.23 (m, 3H), 2.27 (t, 2H, J=7.6 Hz), 2.12-2.00 (m, 3H), 1.91-1.54 (m, 10H), 1.49 (dt, 1H, J=11.9, 5.1 Hz), 1.35-1.23 (m, 12H), 1.20-1.07 (m, 2H), 1.08 (s, 3H), 1.05 (s, 3H), 0.88 (t, 3H, J=6.8 Hz). Elemental Analysis, theoretical (corrected for 0.055% moisture level): C, 81.0%, H, 9.8%, N, 2.8%; found: C, 81.1%, H, 10.2%, N, 2.8%. Differential Scanning Calorimetry (DSC) pattern of this solid shows an endothermic peak with an onset temperature at about 69.0° C., see FIG. 2B.

The abiraterone decanoate obtained in this example was determined to have a purity of 99.7% by weight using a HPLC method. For HPLC analysis, abiraterone decanoate samples were prepared in methanol at a concentration of 0.05 mg/mL (for assay analysis) or 5 mg/mL (for impurity analysis). The HPLC conditions are the following: HPLC column: Halo C8 (2.7 um, 100×3.0 mm); injection volume: 5 uL; Column Temperature: 40° C.; Sample Temperature: ambient; Detection: 210 nm; Mobile Phase: 25 mM Ammonium Acetate, pH 8.0 (MPA) and 95/5 acetonitrile/tetrahydrofuran (MPB); Flow Rate: 0.6 ml/min; Gradient: starting with 65/35 MPA/MPB, in 35 minutes, reaching to 100% MPB, hold at 100% MPB until 40 minutes, at 40.10 minute, back to 65/35 MPA/MPB, and hold at 65/35 MPA/MPB until end at 45 minutes.

The white solid obtained in this example was also characterized by X-Ray Powder Diffraction (XRPD) and Differential Scanning Calorimetry (DSC). XRPD was conducted with Bruker's D8 Discover X-ray diffractometer, with Theta\theta vertical goniometer, using Vantec-500 as detector. Standard conditions: voltage 40 kV, current 40 mA, radiation, Cu, temperature, ambient, X-ray source exit slit size, 0.5 mm pinhole, snout collimator, 0.5 mm, sample holder, ground quartz plate. Operating conditions: detector distance, 30 cm, Chi integration range, 4- to 40-degree 20, count time, 120 seconds/frame, # of frames: 3, Theta 1 position, 4 degree, Theta 2 position, 4 degree, Frame width, 12, scan axis, coupled. Software used include GADDs software, General Area Detector Diffraction System, version 4.1.50; and DIFFRAC.EVA, version 4.0. DSC was performed with TA Instruments Q2000 (Thermal Advantage V 5.0.0—qualified), with a sample size of 2-10 mg, heating range from 25° C. to 250° C. at a heating rate of 10° C./min. Representative XRPD and DSC spectra are shown in FIGS. 2A-2B. A thermogravimetric analysis (TGA) was also performed on this sample. TGA was performed with TA Instruments TGA Q500 (Thermal Advantage V5.2.5—qualified), with a sample size of 5-20 mg, heating range from 25° C. to 150° C. at a heating rate of 10° C./min. A representative TGA trace is shown in FIG. 2C.

### Example 1B. Preparation of High Purity Abiraterone Decanoate

This example shows a process of purifying abiraterone decanoate to remove residue palladium. Abiraterone decanoate used for this Example was prepared using similar procedures as shown in Example 1A.

Crude abiraterone decanoate (7.17 kg) was dissolved in acetone (142 kg) at room temperature. Activated carbon (1.43 kg) was added and the resulting slurry stirred at room temperature for 4 hours. The mixture was filtered to remove the activated carbon and the solids were washed with acetone (142 kg). The combined acetone filtrates were concentrated by vacuum distillation at 40° C. The concentrated filtrates, which contained about 72 L acetone, were then cooled to 20° C. and water (4.3 kg) was slowly added. The mixture was stirred at 20° C. for 12 hours and the abiraterone decanoate was collected by filtration. The product was washed with 1:1 acetone/water (7.2 kg) and dried under vacuum at 40° C. to yield 5.524 kg of pure abiraterone decanoate (Form A). Analytical data are consistent with those described in Example 1A. A representative certificate of analysis of the obtained abiraterone decanoate is shown in Table 1 below.

TABLE 1
Specification and Analysis of Abiraterone Decanoate

| Test Method | Specifications | Result |
|---|---|---|
| Appearance | White to yellow solid | White Solid |
| Identification by IR-ATR | Sample spectrum is | Conforms |
| | consistent with | |
| | Abiraterone Decanoate | |
| | reference standard | |
| | spectrum | |
| Abiraterone Decanoate Content by HPLC (% w/w) | 98-102 (dried basis) | 99.6 |
| Related Substances by HPLC (% w/w) | | |
| Abiraterone | NMT 0.5 | nd |
| Ethylprasterone Decanoate | NMT 0.5 | 0.4 |
| Any Unspecified Impurity | NMT 0.1 | nd |
| Total Impurities | NMT 1.0 | 0.4 |
| Residual Solvents by Gas Chromatography (ppm) | | |
| Dichloromethane | NMT 600 | 181 |
| Acetonitrile | NMT 410 | nd |
| Acetone | NMT 5000 | nd |
| Residual EDU (EDCI coupling reagent by-product) | NMT 0.15 | <0.05 |
| by LC-MS (% w/w) | | |
| Residual Decanoic Acid by Gas Chromatography | NMT 5000 | NT |
| (ppm) | | |
| Triethylamine by Ion Chromatography (% w/w) | NMT 0.5 | <0.1 |
| Palladium by ICP (ppm) | NMT 10 | 3.7 |
| Water by Karl Fischer (% w/w) | NMT 1.0 | 0.12 |
| Residue on Ignition (% w/w) | NMT 0.5 | <0.05 |
| Physical Form by XRPD | Report Results | Crystalline |
| Microbial Enumeration by USP <61> | | |
| Total Aerobic Microbial Count (TAMC) CFU/g | ≤100 | <50 |
| Total Combined Yeasts and Molds (TYMC) CFU/g | ≤10 | <50 |
| Bacterial Endotoxins by USP <85> EU/g | ≤350 | <51.4 |

Notes:
nd = not detected.
NT = Not Tested.
NMT = No More Than

Ethylprasterone Decanoate may be an impurity, which is believed to have the following structure:

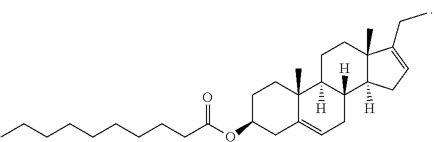

Purity of the obtained abiraterone decanoate was analyzed using a reversed phase HPLC Method. Separation is performed with an Advanced Materials Technology Halo C8 reversed phase column using dimensions of 3.0×100 mm and a particle size of 2.7 μm. A linear gradient program (20 minutes) is used with mobile phases consisting of a 25 mM aqueous ammonium acetate buffer and a mixture of methanol and acetonitrile (see gradient profile below in Table 2). Working standard and sample solutions are prepared in a methanol diluent. The typical injection volume is 5 μL and the detection wavelength is 210 nm.

TABLE 2
Gradient Profile of HPLC Method 1

| | |
|---|---|
| Mobile Phase A: | 25 mM Ammonium Acetate in 45% water, |
| | 45% Acetonitrile and 10% Methanol |
| Mobile Phase B: | 25 mM Ammonium Acetate in 5% Water, |
| | 85% Acetonitrile, and10% Methanol |

| Time (min) | Mobile Phase A (%) | Mobile Phase B (%) |
|---|---|---|
| 0 | 100 | 0 |
| 20 | 0 | 100 |
| 25 | 0 | 100 |
| 25.1 | 100 | 0 |
| 30 | 100 | 0 |

The crude abiraterone decanoate contained 130 ppm Pd. Recrystalization from just acetone/water lowered the Pd level to 120 ppm. However, by using the process described in this example, the final abiraterone decanoate can be purified to have a Pd content of only 3.7 ppm.

### Example 1C. Polymorph Screening of Abiraterone Decanoate

A polymorph screening study was also carried out for abiraterone decanoate. In addition to Form A, as shown in Example 1A, two other polymorphs of abiraterone decanoate were identified, namely Form B and Form C.

Crystallization by cooling at −15° C.: about 30 mg abiraterone decanoate was dissolved in the minimum volume of solvent. Samples were heated at 50° C. for 1 hr if not completely dissolved. Placed samples in a freezer and filtered (if a precipitate was visible) after 2 days. The results using this crystallization method are shown in Table E1 below:

TABLE El
Results from Crystallization by Cooling

| Solvent | Appearance | Form |
|---|---|---|
| Ethanol | White solid | B |
| Dichloromethane | * | * |
| THF | * | * |
| DMF | White solid | A + B |
| Ethyl acetate | White solid | B |
| DMA | White solid | B |
| Isopropyl ether | White solid | A |
| Acetone | White solid | A |
| 1-Propanol | White solid | A + B |
| Chloroform | * | * |
| Trifluoroethanol | * | * |
| 2-Butanol | White solid | A + B |
| Diethyl ether | White solid | A |
| MTBE | White solid | B |
| Corn oil/benzyl alcohol (1:1) | Solvent freezes | * |
| Benzyl alcohol | * | * |
| 2-Propanol | White solid | B |
| Heptane | White solid | B |
| Toluene | * | * |
| Methanol | White solid | B |
| 2-Butanone | White solid | A |
| Acetonitrile | White solid | A + B |

*No solid recovered

Evaporation from binary 1:1 solvent mixtures: about 25 mg abiraterone decanoate dissolved in about 10 mL total volume of solvent. Samples were evaporated under a 1 psi nitrogen purge. The results using this crystallization method are shown in Table E2 below:

TABLE E2
Results from Evaporation from Solvents

| Solvent 1 | Solvent 2 | Appearance | Form |
|---|---|---|---|
| MTBE | Chloroform | Glass | — |
| Solvent 1 | Solvent 2 | Appearance | Form |
| MTBE | Acetone | Glass | — |
| Toluene | Heptane | Glass | — |
| Toluene | Dioxane | Glass | — |
| Dichloromethane | Diethyl ether | Glass | — |
| Methanol | THF | White solid | A + B |
| Methanol | Chloroform | White solid | A + B |
| Ethyl acetate | Ethanol | Glass | — |
| 2-Butanol | Heptane | White solid | B |
| 2-Butanone | Ethanol | White solid | C |
| 2-Butanone | Acetonitrile | White solid | B |
| Acetonitrile | t-Butanol | White solid | A + B |
| Acetonitrile | 2-Propanol | White solid | A + B |
| 1-Propanol | Heptane | Glass | — |
| Acetone | Chloroform | Glass | — |

Anti-solvent addition: about 25 mg abiraterone decanoate dissolved in 1-2 mL solvent, followed by the addition of 2-4 mL of anti-solvent. Samples were filtered if they formed a precipitate. The results using this crystallization method are shown in Table E3 below:

TABLE E3
Results from Anti-Solvent Addition

| Solvent | Anti-solvent | Form |
|---|---|---|
| Dichloromethane | ACN/water | * |
| THF | Water | * |
| Ethanol | Water | A + B |
| Dioxane | Water | * |
| 2-Butanol | Water | * |
| 1-Propanol | Water | A |
| Acetone | Water | * |
| DMA | Water | * |
| Chloroform | ACN/water | A |
| Trifluoroethanol | Water | A |
| t-Butanol | Water | A |
| DMF | Water | * |
| Ethyl acetate | ACN/water | A + B |
| Corn oil/benzyl alcohol (1:1) | ACN/water | * |
| Benzyl alcohol | ACN/water | A + B |
| Benzyl benzoate | ACN/water | * |
| 2-Butanone | Water | A |
| Toluene | ACN/water | * |
| Methanol | Water | A + B |
| 2-Propanol | Water | A |

*Either no precipitate formed or not enough solid recovered

Solvent recrystallization from single solvent: abiraterone decanoate was recrystallized using various solvents. The scale of the recrystallization experiments was approximately 2-10 mL. Saturated solutions were prepared by agitating excess abiraterone decanoate in contact with the various solvent systems at the saturation temperature. If solids did not completely dissolve in the solvent, the mother liquor was separated from the residual solids by filtration. The mother liquor was then heated above the saturation temperature to dissolve any remaining solids. The temperature of each solution was then adjusted to the growth temperature and a controlled nitrogen shear flow was introduced to begin solvent evaporation. The recrystallization conditions for the solvent based panels used during the study are summarized in Tables E4-E5.

TABLE E4
Summary of Fast Evaporation Experiments from
Single Solvent Systems at Ambient Temperature

| Solvent | Form | Solvent | Form |
|---|---|---|---|
| Dioxane | A | 2-Butanol | A |
| 1-Propanol | A | Isopropyl acetate | A + C |
| MTBE | A | Ethanol | A |
| Isopropyl ether | A + C | Water | * |
| t-Butanol | A | Methanol | A |
| Chloroform | A | Toluene | A + C |
| Acetone | A | Acetonitrile | A |
| Ethyl acetate | A | Heptane | A |
| Nitromethane | A | 2-Propanol | A + B |
| DMA | A | 2-Butanone | A |
| THF | A | 2-Methyl THF | A |
| DMF | A | | |
| Diethyl ether | A | | |

*No solids recovered

TABLE E5
Summary of Fast Evaporation Experiments
from Single Solvent Systems at 55° C.

| Solvent | Form | Solvent | Form |
|---|---|---|---|
| Ethyl acetate | A | DMA | Glass |
| 1-Propanol | Glass | 2-Propanol | A |
| Ethanol | Glass | Acetonitrile | A |
| t-Butanol | A | 2-Butanone | A |
| Chloroform | A | Heptane | A |
| Isopropyl ether | A | Toluene | A |
| 2-Butanol | Glass | Methanol | A |
| Dioxane | Glass | Water | * |
| THF | A | 2-Methyl THF | Glass |
| DMF | Glass | | |

*No solids recovered

Non-competitive slurry experiments: The non-competitive slurry experiments were performed by exposing abiraterone decanoate in Form A to solvents and agitating the resulting suspensions for one week at ambient temperature. The solids were filtered and analyzed by XRD to determine the resulting form(s). The solvents used in this study include: water, acetonitrile, isopropyl ether/acetonitrile (1:4), 2-butano/water (1:1), 1-propanol/water (1:1), t-butanol/water (1:1), ethanol/water (1:1), THF/water (1:1), acetone/water (1:1), dioxane/water (1:1), 2-butanone/water (1:1), methanol, DMF/water (1:1), ethyl acetate/water (1:1), and heptane. Based on their X-ray scattering behavior, all of the non-competitive slurry experiments resulted in no change from the starting material.

A competitive study was also carried out. Competitive slurries: about 20 mg abiraterone decanoate suspended in 1-2 mL solvent. Samples were stirred for 3 days and filtered. The results from the competitive studies are shown in Table E6 below:

TABLE E6
Results from Competitive Slurries

| Solvent | Starting Forms | Final Form |
|---|---|---|
| Acetonitrile | A + B | A |
| | A + C | A |
| | B + C | A |
| Methanol | A + B | A |
| | A + C | * |
| | B + C | * |

*No solid recovered—not enough material and solubility too high

Characterization of Forms: Solids generated from the solvent based recrystallization panels were analyzed by powder XRD. To mitigate preferred grain effects, a two dimensional detection system was used to collect all the XRD screening data. The two dimensional detector integrates along the concentric Debye cones which helps reduce pattern variation. If bright spots appear in the conical rings, it indicates strong preferred grain effects that can lead to considerable variability in the observed diffraction patterns including changes in peak intensities. Some samples of abiraterone decanoate exhibited preferred grain effects based on the appearance of the scattering behavior.

The results of this analysis revealed the material exists as at least 3 primary polymorphs. The observed forms were designated as Forms A, B, and C.

After classifying the data into different forms based on diffraction behavior, each form was studied to determine if other properties of the forms could be differentiated. The characterization of each form began by comparing the diffraction data representative of each form with that from the other forms. This was generally followed by NMR, DSC, and TGA.

The initial material used in this study was Form A, which is consistent with the representative characterization data shown in Example 1A, see also summary table E7 below.

Form B was obtained in a variety of crystallization experiments, particularly those carried out at low temperature (−10 to −20° C.). The characteristic diffraction behavior of this form is shown in a representative XRPD spectrum, FIG. 2D. The ¹H NMR spectrum of Form B shows no organic impurities and is consistent with the expected structure of ADEC. Representative DSC and TGA spectra of Form B are shown in FIGS. 2E and 2F.

Form C was obtained by evaporation from a 1:1 mixture of ethanol and 2-butanone. The characteristic diffraction behavior of this form is shown in a representative XRPD spectrum, FIG. 2G. It should be noted that the diffractogram obtained for the one “pure” Form C sample shows significant overlap with Form A at higher diffraction angles and therefore may contain some Form A. The ¹H NMR spectrum of Form C shows no organic impurities and is consistent with the expected structure of ADEC. Representative DSC and TGA spectra of Form C are shown in FIGS. 2H and 2I.

Table E7 below summarizes representative analysis of abiraterone decanoate Forms A, B, and C.

TABLE E7
Results Summary of abiraterone decanoate (ADEC) Forms A, B, and C.

| | | Form | | | | |
|---|---|---|---|---|---|---|
| Technique | Property | A | B | | C | |
| XRD | Crystalline? | Yes | Yes | | Yes* | |
| TGA | Wt % loss @ 150° C. | 0.0 | 0.2 | | 0.0 | |
| DSC | Endotherm | 1^{st} | 1^{st} | 2^{nd} | 1^{st} | 2^{nd} |
| | Onset (° C.) | 67.3 | 60.6 | 64.9 | 58.7 | 66.6 |
| | Peak Max (° C.) | 69.8 | 61.7 | 66.0 | 61.8 | 68.0 |
| | ΔH (J/g) | 80.0 | 36.4 | 36.8 | 51.1 | 3.9 |

| | | |
|---|---|---|
| NMR | Consistent with ADEC structure? | Yes |
| | Residual solvents | Not detected |
| DVS | Max H₂O uptake (wt %) | N/A - non-hygroscopic |

*May contain some Form A

The polymorph screen recrystallization experiments produced either Forms A, B, C, or a mixture of forms. Form A is expected to be thermodynamically stable form under ambient conditions based on the non-competitive and competitive slurry experiments.

### Example 2. Solubility of Abiraterone Decanoate in Different Vehicles

In this example, solubility of abiraterone decanoate in the following four vehicles were tested. Each vehicle has a distinctive base, namely, medium chain trigylceride (MCT)/polyoxylglyceride, long chain (LC) monodiglyceride, and two propylene glycol (PG) monoesters, caprylic and lauryl.
Vehicle 1: MCT/Polyoxylglyceride based: 20% Kolliphor RH40/14% Plurol Oleique CC 497/33% Labrafil 1944 CS/33% Labrafac Lipophile WL 1349
Vehicle 2: PG Monoester based: 20% Kolliphor RH 40/14% Plurol Oleique CC 497/66% Lauroglycol 90
Vehicle 3: PG Monoester based: 20% Kolliphor RH40/14% Plurol Oleique CC 497/66% Capmul PG-8
Vehicle 4: LC Monodiglyceride based: 20% Kolliphor RH40/14% Plurol Oleique CC 497/66% Maisine CC

Solubilities of abiraterone decanoate in different vehicles were tested following the procedure below:
Prepare 5 g of each vehicle by weight
In a 4-mL vial, weigh about 350 mg drug and then add 2 mL of vehicle
Sonicate and vortex all samples, then place on a rotator at 25° C.
At the end of the day check the samples to see if the drug completely dissolved. If so, use wax paper to transfer additional drug, record the additional amount added.
After 2-3 days, remove about 0.5 mL from each sample and filter using a microcentrifuge. Can use 0.45 um filter.
Return the vials to the 25° C. incubator for a later time point
The samples were analyzed for solubility using HPLC methods. Results were obtained for samples after 2 days and after 7 days.

The solubilities of abiraterone decanoate in these vehicles are shown below, which reflect the average of day 2 and day 7 results:

| Vehicle | Solb (mg/g) at 25° C. |
|---|---|
| 1 | 88.5 |
| 2 | 143.9 |
| 3 | 149.2 |
| 4 | 110.2 |

### Example 3. Solubility of Abiraterone Decanoate in Additional Vehicles

This Example tests the solubility of abiraterone decanoate in additional vehicles.

The vehicles being tested have the following compositions:
Vehicle 1 (see Example 2): 20% Kolliphor RH40/14% Plurol Oleique CC 497/33% Labrafil 1944 CS/33% Labrafac Lipophile WL 1349, used as a control.
Vehicle 5: 20% Kolliphor RH40/14% Plurol Oleique CC 497/33% Maisine CC/33% Labrafac Lipophile WL 1349*
Vehicle 6: 20% Kolliphor RH40/14% Plurol Oleique CC 497/16% Labrafil 1944 CS/30% Maisine CC/20% Labrafac Lipophile WL 1349
Vehicle 7: 20% Kolliphor RH40/14% Plurol Oleique CC 497/16% Labrafil 1944 CS/30% Capmul PG-8/20% Labrafac Lipophile WL 1349
Vehicle 8: 20% Kolliphor RH40/14% Plurol Oleique CC 497/16% Labrafil 1944 CS/30% Capmul PG-12/20% Labrafac Lipophile WL 1349
Vehicle 9: 20% Kolliphor RH40/14% Plurol Oleique CC 497/16% Labrafil 1944 CS/30% Labrafil M 2130 CS/20% Labrafac Lipophile WL 1349

The procedures for preparing samples and solubility testing are similar to those described in Example 2. The results are shown in the table below:

Solubility Summary (Day 13 Results)

| | Solubility (mg/g) at |
|---|---|
| Vehicle | 25° C. |
| 1 | 101.1 |
| 5 | 111.5 |
| 6 | 114.3 |
| 7 | 141.2 |
| 8 | 132.9 |

The results from Examples 2 and 3 show that abiraterone decanoate has good solubilities in various lipid based vehicles.

### Example 4. Dispersion of Abiraterone Decanoate Formulations

This example tests dispersibility of abiraterone decanoate formulations in different vehicles. The formulations were prepared using Vehicles 1-4 (shown in Example 2) and Vehicle 9 (Capmul PG-8).

The dispersion tests were conducted following the following general procedure:
Prepare each formulation at an abiraterone decanoate (AbDec) concentration of 10% below solubility in the respective vehicle;
Equilibrate 7.92 mL 0.1 M HCl in vials on rotator at 37° C.;
Add 0.08 mL of the formulation to the 0.1 M HCl medium at 37° C. and start the rotator. To dispense 80 uL, draw dispersant medium up into the pipet and expel several times;
At 60 min, filter the entire 8 mL volume through a 25-mm 0.45 um PVDF filter;
Prepare each formulation for assay by diluting to a concentration below that of the standard using IPA (isopropyl alcohol);
Run HPLC assay on formulation and dispersion samples and a standard;
Also, prepare a sample of Abiraterone in IPA at a similar concentration to AbDec highest standard. This sample will demonstrate if the HPLC method can detect free Ab should it form during the dispersion test.

The results of this dispersion test are shown in the table below:

| | Sample | Actual | |
|---|---|---|---|
| | Conc. | Conc. | % |
| Formulation | (mg/g) | (mg/g) | Recovered |
| AbDec in Vehicle 1 | 81.2 | 84.9 | 95.7 |
| AbDec in Vehicle 2 | 103.5 | 130.0 | 79.6 |
| AbDec in Vehicle 3 | 89.5 | 132.9 | 67.4 |
| AbDec in Vehicle 4 | 8.5 | 99.0 | 8.6 |
| AbDec in Vehicle 9 | 39.4 | 206.8 | 19.0 |

*Actual concentration refers to the concentration of AbDec in the respective vehicle prior to dispersion in the HCl medium

** Sample concentration refers to the concentration of AbDec in the dispersion sample, after considering the dilution factors. The recovery rate was then calculated: (Sample concentration)/(Actual concentration)*100%.

Based on this Example, it was found that abiraterone decanoate in Vehicles 1 and 2 have excellent recovery rate or dispersibility in HCl medium.

### Example 5. Dispersion of Additional Abiraterone Decanoate Formulations

This example tests further abiraterone decanoate formulations in different vehicles. The formulations were prepared using Vehicles 1 (Example 2) and 5-8 (shown in Example 3).

The dispersion tests were conducted following the general procedure shown in Example 4. The results are shown in the table below:

| | AbDec Formulation | % |
|---|---|---|
| Formulation | Conc (mg/g)* | Recovered |
| AbDec in Vehicle 1 | 69.400215 | 121.0 |
| AbDec in Vehicle 5 | 75.206345 | 52.5 |
| AbDec in Vehicle 6 | 76.159275 | 91.9 |
| AbDec in Vehicle 7 | 90.17717 | 96.9 |
| AbDec in Vehicle 8 | 91.68413 | 36.2 |

*This concentration refers to the concentration of abiraterone decanoate in the respective vehicles prior to dispersion.

The results show that the abiraterone decanoate in Vehicles 1, 6, and 7 have excellent recovery rate or dispersibility in HCl medium.

### Example 6. A Single Oral Dose Availability Study of Abiraterone Decanoate in CD®[Crl:CD®(SD)] Rats

The objective of this study is to evaluate the relative absorption of two different oral formulations of abiraterone decanoate over 72 hours after a single oral gavage dose to male rats.

This Example also determines the plasma pharmacokinetics of abiraterone and abiraterone decanoate, serum concentrations of luteinizing hormone and serum concentrations of the steroids androstenedione, corticosterone, progesterone and testosterone in the rat following a single oral administration of abiraterone decanoate to 2 groups of CD® [Crl:CD®(SD)] rats (Group 2, Formulation 1 at 100 mg/kg; Group 3, Formulation 2 at 100 mg/kg). An additional group of animals (Group 1) received the vehicle for formulation 2 and acted as control. In addition tissues were collected from all animals at 72 h post dose and the concentration of abiraterone and abiraterone decanoate determined.

The table below shows the experimental design of this example:

| | | | Dose | Dose | Main Study |
|---|---|---|---|---|---|
| Group | Test | Dose Level | Volume^{a} | Concentration | No. of |
| No. | Material | (mg/kg/day) | (mL/kg) | (mg/mL) | Males |
| 1 | Control Article | 0 | 2.5 | 0 | 4 |
| | (Vehicle 2) | | | | |
| 2 | Abiraterone | 100 | 2.5 | 40 | 4 |
| | decanoate- | | | | |
| | Formulation 1 | | | | |
| 3 | Abiraterone | 100 | 2.5 | 40 | 4 |
| | decanoate- | | | | |
| | Formulation 2 | | | | |

No. = Number
^{a}Based on the most recent body weight measurement.

The details of the test material are the following:
Abiraterone decanoate Formulation 1: 40 mg abiraterone decanoate/mL in Vehicle 1: 20% Kolliphor RH40/14% Plurol Oleique CC497/33% Labrafil 1944CS/33% Labrafac Lipophile WL1349.
Abiraterone decanoate Formulation 2: 40 mg abiraterone decanoate/mL in Vehicle 2: 20% Kolliphor RH40/14% Plurol Oleique CC497/66% Lauroglycol 90.
Control Article is Vehicle 2: 20% Kolliphor RH40/14% Plurol Oleique CC497/66% Lauroglycol 90.

Male rats (strain CD® [Crl:CD®(SD)]) from Charles River Laboratories, Inc. were used for this study.

Plasma and serum samples were collected at 0 min, 1 h, 2 h, 4 h, 24 h, 48 h and 72 h (terminal) postdose for pharmacokinetic or steroid analysis.

The concentrations of abiraterone and abiraterone decanoate in plasma, of luteinizing hormone in serum and of steroids in serum were used to determine, where possible, the following parameters; Tₘₐₓ, Cₘₐₓ, Tₗₐₛₜ, Cₗₐₛₜ, AUCₗₐₛₜ, AUC_{inf}, AUC_{inf} (% extrapolated)

The pharmacokinetic analysis was completed using the software package Phoenix 64 (8.3.1.5014), Certara, Inc.

Tissues (adrenal, brain, femur, liver, lung, mandibular lymph, mesenteric lymph, prostate, sternum and testes) were collected at 72 h post dose and the concentrations at that time are presented.

### Results:

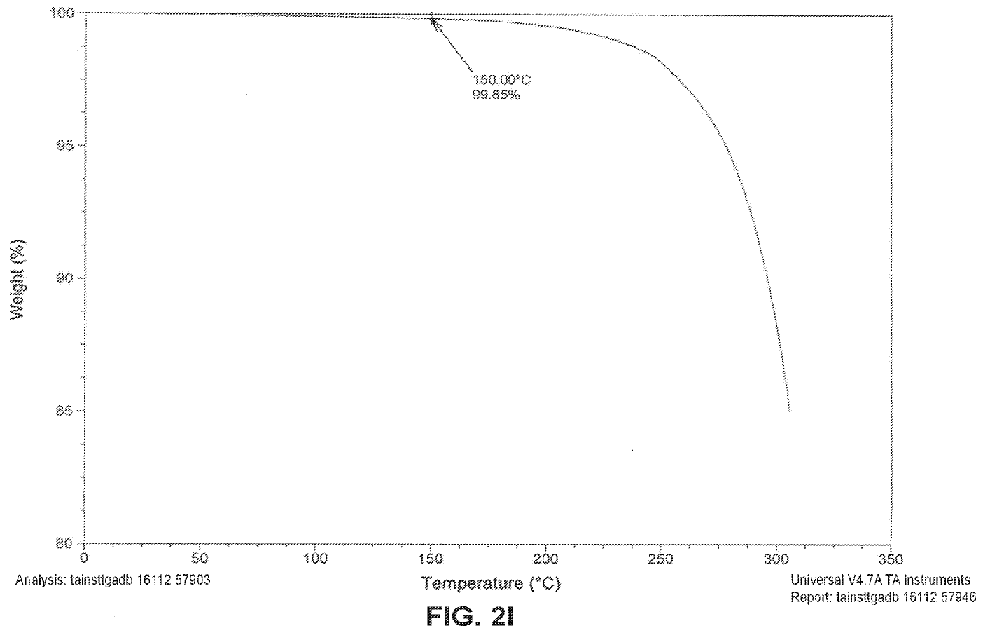

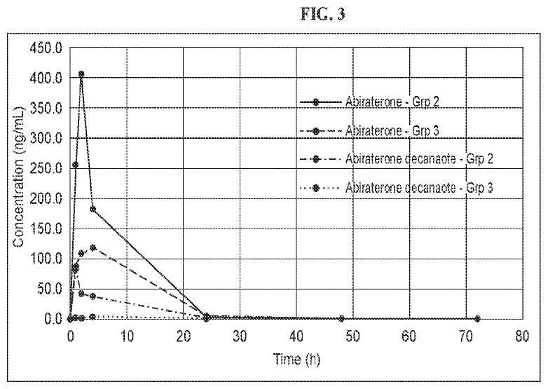

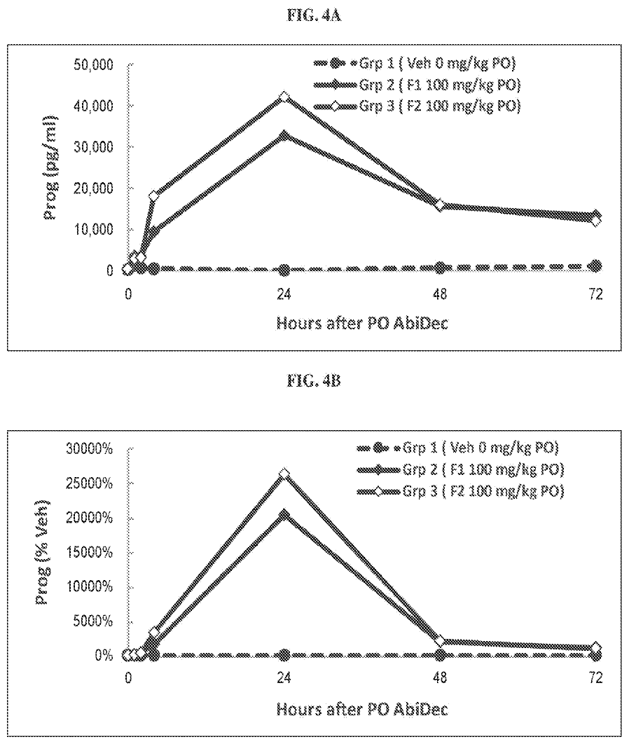

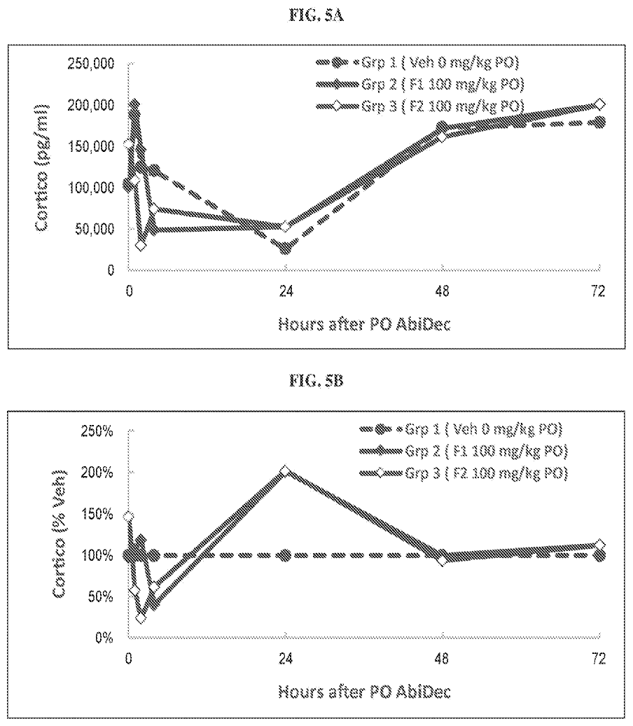

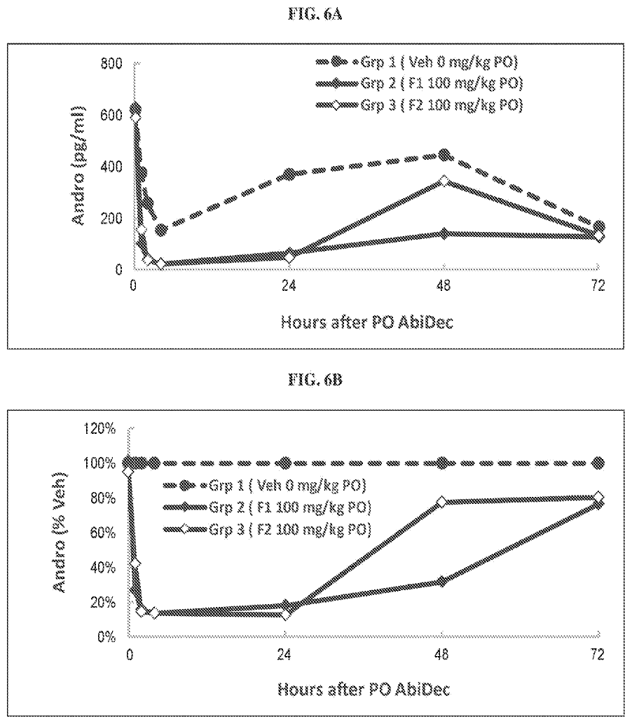

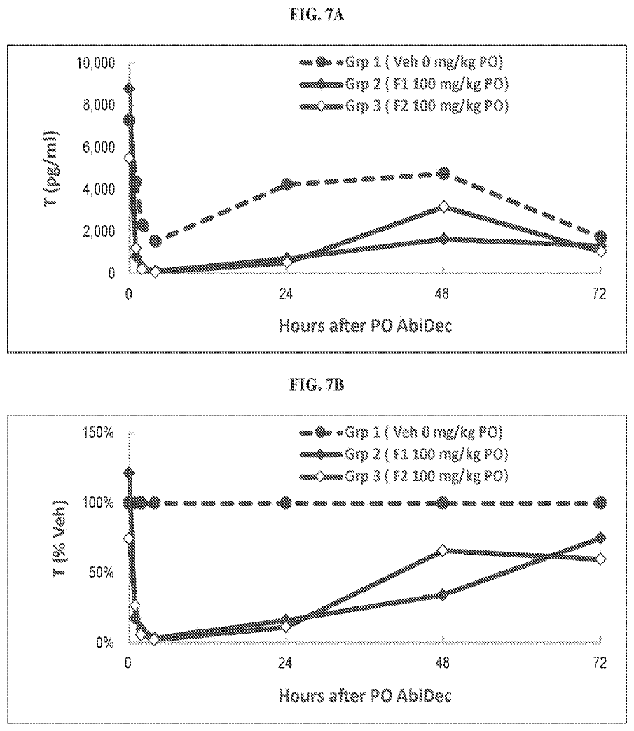

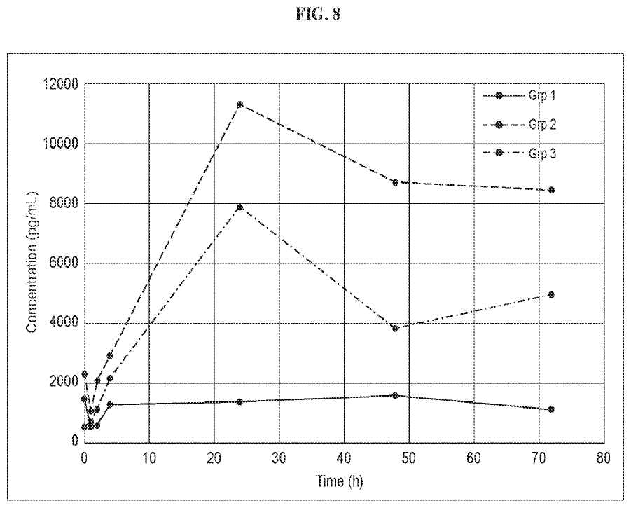

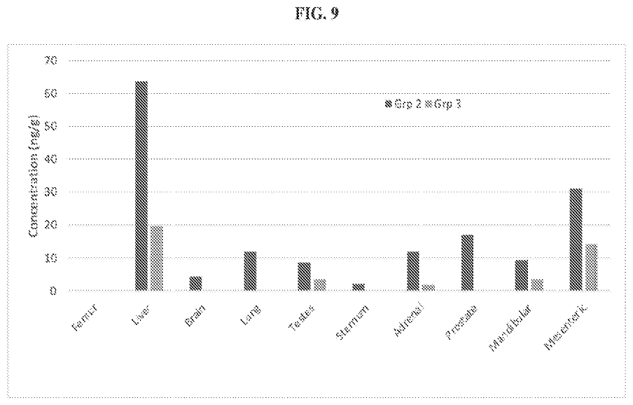

The pharmacokinetic parameters derived from the plasma concentrations of abiraterone and abiraterone decanoate are shown in Tables 3 and 4 and the plasma profile shown in FIG. 3. The effects of oral administration of formulations 1 and 2 on steroid concentrations in plasma are shown graphically in FIGS. 4A, 4B, 5A, 5B, 6A, 6B, 7A, and 7B. The mean plasma concentrations of luteinizing hormone are shown graphically in FIG. 8. The mean and individual tissue concentrations of abiraterone and abiraterone decanoate are shown in Table 5 and Table 6 and shown graphically in FIG. 9.

TABLE 3
Individual and mean pharmacokinetic parameters of
abiraterone following oral administration of abiraterone
decanoate in 2 different formulations

| | | | Cₘₐₓ | | AUCₗₐₛₜ |
|---|---|---|---|---|---|
| Grp | Animal | Tₘₐₓ (h) | (ng/ml) | Tₗₐₛₜ (h) | (ng · h/mL) |
| 2 | 2001 | 2 | 254 | 24 | 2510 |
| | 2002 | 2 | 267 | 72 | 2615 |
| | 2003 | 2 | 620 | 72 | 5004 |
| | 2004 | 2 | 476 | 48 | 3544 |
| Mean | | | 404 | | 3418 |
| SD | | | 176 | | 1155 |
| 3 | 3001 | 1 | 76.1 | 24 | 1066 |
| | 3002 | 2 | 115 | 24 | 1527 |
| | 3003 | 4 | 150 | 24 | 1855 |
| | 3004 | 2 | 172 | 24 | 1914 |
| Mean | | | 128 | | 1591 |
| SD | | | 42.0 | | 389 |

Group 2—Oral abiraterone decanoate at 100 mg/kg in Formulation 1.
Group 3—Oral abiraterone decanoate at 100 mg/kg in Formulation 2.

TABLE 4
Individual and mean pharmacokinetic parameters of abiraterone
decanoate following oral administration of abiraterone
decanoate in 2 different formulations

| | | | Cₘₐₓ | | AUCₗₐₛₜ |
|---|---|---|---|---|---|
| Grp | Animal | Tₘₐₓ (h) | (ng/ml) | Tₗₐₛₜ (h) | (ng · h/mL) |
| 2 | 2001 | 1 | 56 | 4 | 164 |
| | 2002 | 4 | 39.4 | 4 | 113 |
| | 2003 | 1 | 42.6 | 4 | 119 |
| | 2004 | 1 | 79.6 | 4 | 206 |
| Mean | | | 54.4 | | 151 |
| SD | | | 18.3 | | 43.4 |
| 3 | 3001 | 4 | 2.32 | 4 | 4.93 |
| | 3002 | 1 | 2.62 | 4 | 7.59 |
| | 3003 | 4 | 3.2 | 24 | 40.0 |
| | 3004 | 4 | 3.71 | 24 | 49.6 |
| Mean | | | 2.96 | | 25.5 |
| SD | | | 0.618 | | 22.6 |

Group 2—Oral abiraterone decanoate at 100 mg/kg in Formulation 1.
Group 3—Oral abiraterone decanoate at 100 mg/kg in Formulation 2.

TABLE 5
Tissue concentrations of abiraterone following oral administration of abiraterone decanoate in 2 different formulations

| Rat | | Sample | | | | | | | | | | |
|---|---|---|---|---|---|---|---|---|---|---|---|---|
| Number | Group | time (h) | Femur | Liver | Brain | Lung | Testes | Sternum | Adrenal | Prostate | Mandibular | Mesenteric |
| 2001 | 2 | 72 | BLQ | 30.6 | BLQ | 10.2 | 11.3 | 2.37 | 13.8 | 16.2 | 7.56 | 25.1 |
| 2002 | 2 | 72 | BLQ | 84.6 | 6.48 | 11.1 | 10.2 | BLQ | 9.15 | 16.0 | 7.74 | 17.4 |
| 2003 | 2 | 72 | BLQ | 120 | 10.6 | 18.5 | 7.05 | 6.23 | 13.1 | 23.6 | 12.6 | 54.0 |
| 2004 | 2 | 72 | BLQ | 19.5 | BLQ | 7.47 | 6.06 | BLQ | 11.1 | 12.3 | <6 | 27.4 |
| Mean | | | BLQ | 63.6 | 8.6 | 11.8 | 8.7 | 2.2 | 11.8 | 17.0 | 9.3 | 31.0 |
| SD | | | NA | 47.0 | 2.9 | 4.7 | 2.5 | 2.9 | 2.1 | 4.7 | 2.8 | 15.9 |
| 3001 | 3 | 72 | BLQ | 16.0 | BLQ | BLQ | BLQ | BLQ | BLQ | BLQ | 7.98 | 20.3 |
| 3002 | 3 | 72 | BLQ | 12.7 | BLQ | BLQ | BLQ | BLQ | BLQ | BLQ | BLQ | BLQ |
| 3003 | 3 | 72 | BLQ | 30.0 | BLQ | BLQ | 6.24 | BLQ | BLQ | BLQ | BLQ | 17.7 |
| 3004 | 3 | 72 | BLQ | 20.3 | BLQ | BLQ | 8.55 | BLQ | 7.86 | BLQ | 6.09 | 18.9 |
| Mean | | | BLQ | 19.7 | BLQ | BLQ | 3.7 | BLQ | 2.0 | BLQ | 3.5 | 14.2 |
| SD | | | NA | 7.5 | NA | NA | 4.4 | NA | NA | NA | 4.1 | 9.5 |

BLQ—Femur <5.5 ng/g, brain <6 ng/g, lung <6 ng/g, sternum <1.1 ng/mL, adrenal <6 ng/g, prostate <6 ng/g, mandibular lymph node <6 ng/g, mesenteric lymph node <6 ng/g.
BLQ values taken as zero for determination of the mean.
Group 2—Oral abiraterone decanoate at 100 mg/kg in Formulation 1.
Group 3—Oral abiraterone decanoate at 100 mg/kg in Formulation 2.

TABLE 6
Tissue concentrations of abiraterone decanoate following oral administration
of abiraterone decanoate in 2 different formulations

| Rat | | Sample | | | | | | | | | | |
|---|---|---|---|---|---|---|---|---|---|---|---|---|
| Number | Group | time (h) | Femur | Liver | Brain | Lung | Testes | Sternum | Adrenal | Prostate | Mandibular | Mesenteric |
| 2001 | 2 | 72 | BLQ | BLQ | BLQ | BLQ | BLQ | 1.11 | BLQ | BLQ | BLQ | BLQ |
| 2002 | 2 | 72 | BLQ | BLQ | BLQ | BLQ | BLQ | BLQ | BLQ | BLQ | BLQ | BLQ |
| 2003 | 2 | 72 | BLQ | BLQ | BLQ | BLQ | BLQ | BLQ | BLQ | BLQ | BLQ | BLQ |
| 2004 | 2 | 72 | BLQ | BLQ | BLQ | BLQ | BLQ | BLQ | BLQ | BLQ | BLQ | BLQ |
| Mean | | | BLQ | BLQ | BLQ | BLQ | BLQ | 0.3 | BLQ | BLQ | BLQ | BLQ |
| SD | | | NA | NA | NA | NA | NA | NA | NA | NA | NA | NA |
| 3001 | 3 | 72 | BLQ | BLQ | BLQ | BLQ | BLQ | BLQ | BLQ | BLQ | BLQ | BLQ |
| 3002 | 3 | 72 | BLQ | BLQ | BLQ | BLQ | BLQ | BLQ | BLQ | BLQ | BLQ | BLQ |
| 3003 | 3 | 72 | BLQ | BLQ | BLQ | BLQ | BLQ | BLQ | BLQ | BLQ | BLQ | BLQ |
| 3004 | 3 | 72 | BLQ | BLQ | BLQ | BLQ | BLQ | BLQ | BLQ | BLQ | BLQ | BLQ |
| Mean | | | BLC | BLC | BLQ | BLQ | BLQ | BLQ | BLQ | BLQ | BLQ | BLC |
| SD | | | NA | NA | NA | NA | NA | NA | NA | NA | NA | NA |

BLQ—Femur <5.5 ng/g, liver <1.5 ng/g, brain <1.5 ng/g, lung <1.5 ng/g, testes <1.5 ng/g, sternum <1.5 ng/ml, adrenal <1.5 ng/g, prostate <1.5 ng/g, mandibular lymph node <1.5 ng/g, mesenteric lymph node <1.5 ng/g.
BLQ values taken as zero for determination of the mean.
Group 2—Oral abiraterone decanoate at 100 mg/kg in Formulation 1.
Group 3—Oral abiraterone decanoate at 100 mg/kg in Formulation 2.

### Plasma Concentration of Abiraterone and Abiraterone Decanoate:

Following single oral administration of Formulation 1 the Cₘₐₓ for abiraterone was 404 ng/mL, Tₘₐₓ was at 2 h and the AUCₗₐₛₜ was 3418 ng·h/mL. Following single oral administration of formulation 2 the Cₘₐₓ of abiraterone was 128 ng/mL, Tₘₐₓ was between 1 to 4 h and the AUCₗₐₛₜ was 1591 ng·h/mL. From this, the relative exposure of abiraterone from an oral dose of Formulation 1 was 3.2-fold and 2.1-fold higher for Cₘₐₓ and AUCₗₐₛₜ, respectively than for Formulation 2. The oral bioavailability based on plasma concentrations of abiraterone for Formulations 1 and 2 are about 59% and 27%, based on the following IV data: IV AbiDec 1.2 mg/kg: Abi AUC=69.8 ng·h/mL (equivalent to 5817 ng·h/ml for a 100 mg/kg dose).

Following single oral administration of Formulation 1, the Cₘₐₓ for abiraterone decanoate was 54.4 ng/mL, Tₘax was at 1 to 4 h and the AUCₗₐₛₜ was 151 ng·h/mL. Following single oral administration of formulation 2 the Cₘₐₓ of abiraterone decanoate was 2.96 ng/mL, Tₘax was between 1 to 4 h and the AUCₗₐₛₜ was 25.5 ng·h/mL. From this, the relative exposure of abiraterone decanoate from an oral dose of Formulation 1 was 18.3-fold and 5.92-fold higher for Cₘₐₓ and AUCₗₐₛₜ, respectively than for Formulation 2.

### Tissue Concentrations of Abiraterone and Abiraterone Decanoate:

At 72 h, following oral administration of Formulation 1, abiraterone was detected in all tissues except for femur with levels tending to be relatively low, with the highest concentration in liver (63.6 ng/g) and mesenteric lymph node (31.0 ng/g). Following oral administration of Formulation 2 concentrations of abiraterone were lower or BLQ in all tissues, with the highest concentrations in liver (19.7 ng/g) and mesenteric lymph node (14.2 ng/g), consistent with the results for Formulation 1.

### Discussions:

In this study there was a comparison of 2 different formulations of abiraterone decanoate for oral administration. In the rat, exposure of abiraterone and abiraterone decanoate was higher following oral administration of Formulation 1 indicating a better relative absorption compared to Formulation 2.

Both formulations reduced the plasma concentration of androstenedione, increased the concentration of progesterone and reduced the concentration of testosterone. There was no effect on the concentration of corticosterone. The effect of each formulation was considered similar (<2-fold difference) but the effect of Formulation 1 tended to be greater than for Formulation 2 consistent with the higher exposure to abiraterone.

Consistent with the effect on steroids the exposure to luteinizing hormone was increased by both Formulations, with the exposure tending to be higher for Formulation 1 than 2.

Abiraterone but not abiraterone decanoate was detected in tissues at 72 h post dose, with highest concentrations in liver and mesenteric lymph node, with higher concentrations from Formulation 1 than Formulation 2.

Each reference referred to within this disclosure is hereby incorporated in its respective entirety.

With respect to aspects of the disclosure described as a genus, all individual species are individually considered separate aspects of the disclosure. If aspects of the disclosure are described as “comprising” a feature, embodiments also are contemplated “consisting of” or “consisting essentially of” the feature.

All the various aspects, embodiments, and options described herein can be combined in any and all variations.

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, because numerous modifications will readily occur to those skilled in the art, it is not desired to limit the invention to the exact construction and operation illustrated and described, and accordingly, all suitable modifications and equivalents may be resorted to, falling within the scope of the invention.

What is claimed is:

1. A pharmaceutical composition comprising: (a) abiraterone decanoate; and (b) a lipid-based drug delivery system, wherein abiraterone decanoate has the following structure: 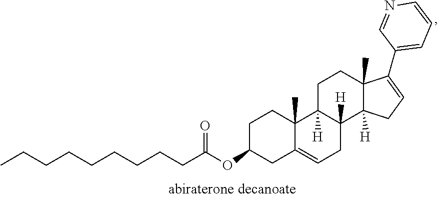
wherein the pharmaceutical composition is formulated for oral delivery of abiraterone decanoate.

2. The pharmaceutical composition of claim 1, wherein the lipid-based drug delivery system comprises: (1) a triglyceride, monoglyceride, diglyceride, and/or a propylene glycol ester; and (2) a surfactant comprising a polyglyceryl ester and/or polyoxyglyceride.

3. The pharmaceutical composition of claim 1 or 2, wherein the lipid-based drug delivery system comprises a triglyceride.

4. The pharmaceutical composition of claim 1 or 2, wherein the lipid-based drug delivery system comprises a medium-chain triglyceride (e.g., Labrafac™ lipophile WL 1349, or medium-chain triglycerides of caprylic (C8) and capric (C10) acids).

5. The pharmaceutical composition of any one of claims 1-4, wherein the lipid-based drug delivery system comprises a monoglyceride and/or diglyceride.

6. The pharmaceutical composition of any one of claims 1-4, wherein the lipid-based drug delivery system comprises a glycerol/glyceryl linoleate (e.g., Maisine® CC, mono-, di- and triglycerides of mainly linoleic (C_{18:2}) and oleic (C_{18:1}) acids, the diester fraction being predominant).

7. The pharmaceutical composition of any one of claims 1-6, wherein the lipid-based drug delivery system comprises a propylene glycol ester.

8. The pharmaceutical composition of any one of claims 1-6, wherein the lipid-based drug delivery system comprises propylene glycol monocaprylate (e.g., Capmul PG-8) and/or propylene glycol monolaurate (e.g., Capmul PG-12, or Lauroglycol™ 90).

9. The pharmaceutical composition of any one of claims 1-8, wherein the lipid-based drug delivery system comprises a surfactant comprising a polyglycerol ester.

10. The pharmaceutical composition of any one of claims 1-8, wherein the lipid-based drug delivery system comprises a surfactant comprising polyglyceryl oleate (e.g., Plurol Oleique CC 497 (polyglyceryl-3 dioleate)).

11. The pharmaceutical composition of any one of claims 1-10, wherein the lipid-based drug delivery system comprises a surfactant comprising a polyoxyglyceride.

12. The pharmaceutical composition of any one of claims 1-10, wherein the lipid-based drug delivery system comprises a surfactant comprising macrogolglycerol hydroxystearate (e.g., Kolliphor RH 40), oleoyl polyoxyl-6 glycerides (e.g., Labrafil® M 1944 CS) or lauroyl polyoxyl-6 glycerides (e.g., Labrafil 2130).

13. The pharmaceutical composition of any one of claims 1-12, wherein the abiraterone decanoate is dispersed, such as homogeneously dispersed or dissolved, in the lipid-based drug delivery system, with a concentration ranging from about 1 mg/g to about 250 mg/g, e.g., about 20 mg/g to about 150 mg/g.

14. The pharmaceutical composition of any one of claims 1-13, formulated in the form of an oral dosage form, such as a capsule (e.g., a soft gel capsule).

15. The pharmaceutical composition of any one of claims 1-14, which is characterized by one or more of the following: (1) the pharmaceutical composition is storage stable at room temperature; (2) the recovery of abiraterone decanoate is greater than 50% when the pharmaceutical composition is assessed using an in vitro dispersion test; and (3) upon oral administration to a mammal, the pharmaceutical composition is capable of delivering a sufficient amount of abiraterone decanoate to the mammal to achieve a therapeutically effective plasma concentration of abiraterone, e.g., for treating a disease or disorder described herein, such as a prostate cancer described herein.

16. The pharmaceutical composition of any one of claims 1-14, which has an oral bioavailability greater than 30% based on abiraterone plasma concentration profile, when tested in rats.

17. A pharmaceutical composition comprising abiraterone decanoate dissolved in a lipid-based drug delivery system at a concentration ranging from about 10 mg/g to about 150 mg/g, wherein the lipid-based drug delivery system comprises (a) a lipid in an amount of about 10-80% by weight of the lipid-based drug delivery system; and (b) one or more non-ionic surfactants in an amount of about 20-90% by weight of the lipid-based drug delivery system, wherein abiraterone decanoate has the following structure:

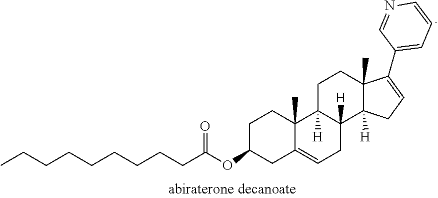

18. The pharmaceutical composition of claim 17, wherein the lipid comprises medium-chain triglycerides of caprylic (CS) and capric (C10) acids (e.g., Labrafac™ lipophile WL 1349) in an amount of about 10% to about 50% by weight of the lipid-based drug delivery system, such as about 20-40% by weight.

19. The pharmaceutical composition of claim 17 or 18, wherein the lipid comprises glycerol/glyceryl linoleate (e.g., Maisine® CC) in an amount of about 10% to about 50% by weight of the lipid-based drug delivery system, such as about 20-40% by weight.

20. The pharmaceutical composition of any of claims 17-19, wherein the lipid further comprises propylene glycol monocaprylate (e.g., Capmul PG-8) or propylene glycol monolaurate (e.g., Capmul PG-12, or Lauroglycol™ 90) in an amount of about 10% to about 50% by weight of the lipid-based drug delivery system, such as about 20-40% by weight.

21. The pharmaceutical composition of any of claims 17-20, wherein the lipid-based drug delivery system comprises two or more, such as two or three, non-ionic surfactants.

22. The pharmaceutical composition of any of claims 17-21, wherein the one or more non-ionic surfactants comprise macrogolglycerol hydroxystearate (e.g., Kolliphor RH 40) and/or polyglyceryl oleate (e.g., Plurol Oleique CC 497 (polyglyceryl-3 dioleate)).

23. The pharmaceutical composition of any of claims 17-22, wherein the one or more non-ionic surfactants further comprise oleoyl polyoxyl-6 glycerides (e.g., Labrafil® M 1944 CS) and/or lauroyl polyoxyl-6 glycerides (e.g., Labrafil 2130).

24. The pharmaceutical composition of any of claims 17-23, which comprises abiraterone decanoate dissolved in the lipid-based drug delivery system at a concentration ranging from about 20 mg/g to about 120 mg/g, wherein the lipid-based drug delivery system comprises (a) medium-chain triglycerides of caprylic (C8) and capric (C10) acids in an amount of about 20-40% by weight of the lipid-based drug delivery system; (b) macrogolglycerol hydroxystearate (e.g., Kolliphor RH 40) in an amount of about 10-30% by weight of the lipid-based drug delivery system; (c) polyglyceryl oleate (e.g., Plurol Oleique CC 497 (polyglyceryl-3 dioleate)) in an amount of about 10-30% by weight of the lipid-based drug delivery system; and (d) oleoyl polyoxyl-6 glycerides (e.g., Labrafil® M 1944 CS) in an amount of about 20-40% by weight of the lipid-based drug delivery system.

25. The pharmaceutical composition of any of claims 17-23, which comprises abiraterone decanoate dissolved in the lipid-based drug delivery system at a concentration ranging from about 20 mg/g to about 120 mg/g, wherein the lipid-based drug delivery system comprises (a) medium-chain triglycerides of caprylic (C8) and capric (C10) acids in an amount of about 10-40% by weight of the lipid-based drug delivery system; (b) macrogolglycerol hydroxystearate (e.g., Kolliphor RH 40) in an amount of about 10-30% by weight of the lipid-based drug delivery system; (c) polyglyceryl oleate (e.g., Plurol Oleique CC 497 (polyglyceryl-3 dioleate)) in an amount of about 10-30% by weight of the lipid-based drug delivery system; (d) oleoyl polyoxyl-6 glycerides (e.g., Labrafil® M 1944 CS) in an amount of about 10-40% by weight of the lipid-based drug delivery system; and (e) propylene glycol monocaprylate (e.g., Capmul PG-8) and/or propylene glycol monolaurate (e.g., Capmul PG-12, or Lauroglycol™ 90) in an amount of about 10-40% by weight of the lipid-based drug delivery system.

26. The pharmaceutical composition of any of claims 17-23, which comprises abiraterone decanoate dissolved in the lipid-based drug delivery system at a concentration ranging from about 20 mg/g to about 120 mg/g, wherein the lipid-based drug delivery system comprises (a) medium-chain triglycerides of caprylic (C8) and capric (C10) acids in an amount of about 10-40% by weight of the lipid-based drug delivery system; (b) macrogolglycerol hydroxystearate (e.g., Kolliphor RH 40) in an amount of about 10-30% by weight of the lipid-based drug delivery system; (c) polyglyceryl oleate (e.g., Plurol Oleique CC 497 (polyglyceryl-3 dioleate)) in an amount of about 10-30% by weight of the lipid-based drug delivery system; (d) oleoyl polyoxyl-6 glycerides (e.g., Labrafil® M 1944 CS) in an amount of about 0-40% by weight of the lipid-based drug delivery system; and (e) glycerol/glyceryl linoleate (e.g., Maisine® CC, mono-, di- and triglycerides of mainly linoleic (C_{18:2}) and oleic (C_{18:1}) acids, the diester fraction being predominant) in an amount of about 10-40% by weight of the lipid-based drug delivery system.

27. The pharmaceutical composition of claim 17, which comprises a vehicle described in any of the examples herein.

28. The pharmaceutical composition of any one of claims 17-27, formulated for oral administration, such as in the form of a capsule (e.g., a soft gel capsule).

29. The pharmaceutical composition of any one of claims 17-25, which is characterized by one or more of the following: (1) the pharmaceutical composition is storage stable at room temperature; (2) the recovery of abiraterone decanoate is greater than 50% when the pharmaceutical composition is assessed using an in vitro dispersion test; and (3) upon oral administration to a mammal, the pharmaceutical composition is capable of delivering a sufficient amount of abiraterone decanoate to the mammal to achieve a therapeutically effective plasma concentration of abiraterone, e.g., for treating a disease or disorder described herein, such as a prostate cancer described herein.

30. The pharmaceutical composition of any one of claims 17-29, which has an oral bioavailability of greater than 30% based on abiraterone plasma concentration profile, when tested in rats.

31. The pharmaceutical composition of any one of claims 1-30, wherein the lipid-based drug delivery system is a self-dispersing drug delivery system, such as a self-emulsifying drug delivery system or self-microemulsifying drug delivery system.

32. The pharmaceutical composition of any one of claims 1-31, wherein upon oral administration to a mammal, at least a portion of the abiraterone decanoate is absorbed through the lymphatic system.

33. The pharmaceutical composition of any one of claims 1-32, wherein the abiraterone decanoate is substantially pure, e.g., characterized as having a purity by weight of at least 95%, preferably, at least 98%, such as about 98.5%, about 99%, about 99.5%, or higher.

34. The pharmaceutical composition of claim 33, wherein the abiraterone decanoate is characterized as having less than 1% (e.g., less than 0.5% by weight, such as less than 0.3%, less than 0.2%, or less than 0.1%) by weight of ethyl prasterone decanoate having the formula:

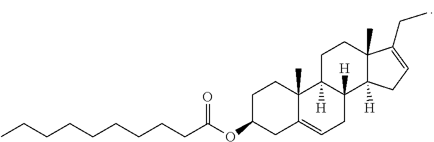

35. The pharmaceutical composition of claim 33, wherein the abiraterone decanoate is characterized as having no detectable amount of ethyl prasterone decanoate.

36. The pharmaceutical composition of any one of claims 33-35, wherein the abiraterone decanoate is characterized as having a Palladium content of less than 50 ppm.

37. The pharmaceutical composition of any one of claims 33-35, wherein the abiraterone decanoate is characterized as having a Palladium content of less than 10 ppm.

38. A method of treating or preventing a disease or disorder in a subject in need thereof, the method comprising administering to the subject an effective amount of the pharmaceutical composition according to any one of claims 1-37, wherein the disease or disorder is selected from a sex hormone-dependent benign or malignant disorder, an androgen receptor driven cancer, a syndrome due to androgen excess, and a syndrome due to glucocorticoid excess.

39. The method of claim 38, wherein the disease or disorder is selected from prostate cancer, breast cancer, endometrial cancer, ovarian cancer, bladder cancer, hepatocellular carcinoma, lung cancer, endometriosis, polycystic ovary syndrome, Cushing's syndrome, Cushing's disease, classical or nonclassical congenital adrenal hyperplasia, precocious puberty, hirsutism, and combinations thereof.

40. The method of claim 38, wherein the disease or disorder is a sex hormone dependent or androgen receptor driven cancer.

41. The method of claim 40, wherein the sex hormone dependent or androgen receptor driven cancer is androgen receptor positive salivary duct carcinoma, or androgen receptor positive glioblastoma multiforme.

42. The method of claim 38, wherein the disease or disorder is prostate cancer.

43. The method of claim 42, wherein the subject having prostate cancer is characterized as having a rising amount of prostate specific antigen, e.g., following radical prostatectomy.

44. The method of claim 42, wherein the prostate cancer is a localized prostate cancer, e.g., a high risk localized prostate cancer.

45. The method of claim 42, wherein the prostate cancer is a metastatic castration-sensitive prostate cancer, non-metastatic castration-sensitive prostate cancer, non-metastatic castration-resistant prostate cancer, or metastatic castration-resistant prostate cancer.

46. The method of claim 42, wherein the prostate cancer is a newly diagnosed high risk metastatic hormone sensitive prostate cancer.

47. The method of claim 42, wherein the prostate cancer is a metastatic castration resistant prostate cancer (mCRPC), wherein the subject is asymptomatic or mildly symptomatic after failure of androgen deprivation therapy in whom chemotherapy is not yet clinically indicated.

48. The method of claim 42, wherein the prostate cancer is a metastatic castration resistant prostate cancer (mCRPC), wherein the subject's disease has progressed on or after a taxane-based such as docetaxel-based chemotherapy regimen.

49. The method of claim 42, wherein the prostate cancer is a refractory prostate cancer.

50. The method of any one of claims 38-49, further comprising treating the subject with radiotherapy or surgery.

51. The method of any one of claims 38-50, further comprising administering to the subject one or more other agents selected from anticancer agents, hormone ablation agents, anti-androgen agents, differentiating agents, anti-neoplastic agents, kinase inhibitors, anti-metabolite agents, alkylating agents, antibiotic agents, immunological agents, interferon-type agents, intercalating agents, growth factor inhibitors, cell cycle inhibitors, enzymes, topoisomerase inhibitors, biological response modifiers, mitotic inhibitors, matrix metalloprotease inhibitors, genetic therapeutics, or combinations thereof.

52. The method of any one of claims 38-51, further comprising administering to the subject one or more agents selected from hydrocortisone, prednisone, prednisolone, methylprednisolone, and dexamethasone.

53. The method of any one of claims 38-52, further comprising administering to the subject one or more other agents selected from a chemotherapeutic drug, hormone replacement drug, or hormone ablation drug.

54. The method of any one of claims 38-53, further comprising treating the subject with an androgen deprivation therapy.

55. The method of any one of claims 38-53, wherein the subject is a non-castrated subject.

56. The method of any one of claims 38-53, wherein the subject is not treated with a gonadotropin-releasing hormone agonist and/or antagonist in an amount effective to reduce serum testosterone level in the subject.

57. The method of claim 56, wherein the subject is not treated with a drug selected from buserelin, leuprolide, deslorelin, fertirelin, histrelin, gonadorelin, lecirelin, goserelin, nafarelin, peforelin and triptorelin.

58. The method of claim 56, wherein the subject is not treated with a drug selected from abarelix, cetrorelix, degarelix, ganirelix, elagolix, linzagolixa, and relugolix.

59. The method of any one of claims 55-58, wherein the subject is sensitive to or otherwise intolerant with a gonadotropin-releasing hormone antagonist and/or agonist.

60. The method of any one of claims 55-59, wherein the subject is not treated with a glucocorticoid replacement therapy.

61. The method of any one of claims 38-60, further comprising administering to the subject a poly ADP ribose polymerase (PARP) inhibitor, e.g., niraparib, rucaparib, olaparib, talazoparib, veliparib, and fluzoparib.

62. The method of any one of claims 38-61, further comprising administering to the subject a 1^{st}-generation androgen receptor antagonist, e.g., proxalutamide, bicalutamide, flutamide, nilutamide, topilutamide.

63. The method of any one of claims 38-62, further comprising administering to the subject a 2^{nd}-generation androgen receptor antagonist (e.g., apalutamide, darolutamide or enzalutamide).

64. The method of any one of claims 38-63, further comprising administering to the subject a 3^{rd} generation androgen receptor antagonist (such as an N-terminal domain inhibitor) or an androgen receptor degrader molecule, alone or in combination with one or more 1^{st} generation or 2^{nd} generation androgen receptor antagonists.

65. The method of any one of claims 38-64, further comprising administering to the subject a chemotherapeutic agent, such as a taxane based chemotherapeutic agent (e.g., docetaxel, cabazitaxel, paclitaxel, etc.) or platinum based chemotherapeutic agent (e.g., cisplatin, carboplatin, oxaliplatin, etc.).

66. The method of any one of claims 38-65, further comprising administering to the subject an immunotherapy, such as administering Sipuleucel-T, an immune checkpoint inhibitor (e.g., anti-PD-1 antibody such as pembrolizumab or nivolumab, or anti-PD-L1 antibody such as avelumab or atezolizumab), or an anti-CTLA-4 antibody (e.g., ipilimumab), etc.

67. The method of any one of claims 38-66, further comprising administering to the subject a bispecific T-cell engager (BiTE) therapy, such as blinatumomab or solitomab.

68. The method of any one of claims 38-67, further comprising administering to the subject a kinase inhibitor, e.g., sunitinib, dasatinib, cabozantinib, erdafitinib, dovitinib, capivasertib, onvansertib, ipatasertib, afuresertib, alisertib, apitolisib, opaganib, etc.

69. The method of any one of claims 38-68, further comprising administering to the subject a bone protecting agent (e.g., denosumab, zolendronic acid), and wherein the subject is characterized as having prostate cancer (e.g., CRPC) with bone metastasis.

70. The method of any one of claims 38-69, further comprising administering to the subject a therapeutic agent selected from 1) an anti-IL23 targeting monoclonal antibody, e.g., tildrakizumab; 2) a selenium, such as sodium selenite; 3) an EZH2 inhibitor, e.g., CPI-1205, GSK2816126, or tazemetostat; 4) a CDK4/6 inhibitor, e.g., palbociclib, ribociclib, abemaciclib; 6) a bromodomain and extra-terminal domain (BET) inhibitor, e.g., CCS1477, INCB057643, alobresib, ZEN-3694, or molibresib (GSK525762); 7) an anti-CD105 antibody, e.g., TRC105 or carotuximab; 8) niclosamide; 9) an A2A receptor antagonist, e.g., AZD4635; 10) a PI3K inhibitor, e.g., AZD-8186, buparlisib, or dactolisib; 11) a further non-steroidal CYP17A1 inhibitor, e.g. seviteronel; 12) an antiprogestogen, e.g., onapristone; 13) navitoclax; 14) an HSP90 inhibitor, e.g., onalespib (AT13387); 15) an HSP27 inhibitor, e.g., OGX-427; 16) a 5-alpha-reductase inhibitor, e.g., dutasteride; 17) metformin; 18) AMG-386; 19) dextromethorphan; 20) theophylline; 21) hydroxychloroquine; and 22) lenalidomide.

71. The method of any one of claims 38-70, further comprising administering to the subject one or more kinase modulators selected from FLT-3 (FMS-like tyrosine kinase) inhibitors, AXL (anexelekto) inhibitors (e.g., Gilteritinib), CDK (cyclin dependent kinase) inhibitors, such as CDK1, 2, 4, 5, 6, 7, or 9 inhibitors, retinoblastoma (Rb) inhibitors, protein kinase B (AKT) inhibitors, SRC inhibitors, IkappaB kinase 1 (IKK1) inhibitors, PIM-1 modulators, Lemur tyrosine kinase 2 (LMTK2) modulators, Lyn inhibitors, Aurora A inhibitors, ANPK (a nuclear protein kinase) inhibitors, extracellular-signal regulated kinase (ERK) modulators, c-jun N-terminal kinase (JNK) modulators, Big MAP kinase (BMK) modulators, p38 mitogen-activated protein kinases (MAPK) modulators, and combinations thereof.

72. The method of any one of claims 38-71, wherein the subject is chemotherapy naïve or hormone therapy naïve prior to being administered the pharmaceutical composition.

73. The method of any one of claims 38-72, wherein the subject has not undergone a prostatectomy.

74. The method of any one of claims 38-73, wherein the subject is treated with radiotherapy e.g., stereotactic body radiotherapy, neutron radiation.

75. The method of any one of claims 38-74, wherein the subject is administered Radium-223.

76. The method of claim 38, wherein the disease or disorder is breast cancer, e.g., molecular apocrine HER2-negative breast cancer, metastatic breast cancer, such as ER+ metastatic breast cancer, ER+ and HER2 negative breast cancer, AR+ triple negative breast cancer, etc.

77. The method of claim 76, further comprising administering to the subject an aromatase inhibitor, e.g., exemestane.

78. The method of claim 38, wherein the disease or disorder is associated with 21-hydroxylase deficiency.

79. The method of any one of claims 38-78, wherein the pharmaceutical composition is administered orally.

80. The method of any one of claims 38-79, wherein the pharmaceutical composition is administered to the subject ranging from once a day to once a week, such as once a day or once every two or three days.

81. The method of any one of claims 38-80, wherein the pharmaceutical composition is administered to the subject with or without food.

82. An emulsion comprising (a) abiraterone decanoate; (b) a lipid; and (c) a non-ionic surfactant, wherein the lipid phase of the emulsion comprises abiraterone decanoate dispersed in the lipid, wherein abiraterone decanoate has the following structure:

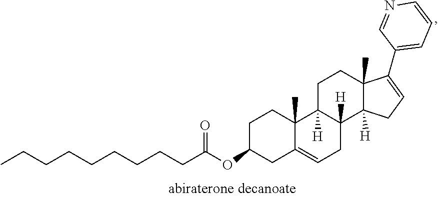

83. The emulsion of claim 82, wherein the lipid comprises medium-chain triglycerides of caprylic (C8) and capric (C10) acids (e.g., Labrafac™ lipophile WL 1349).

84. The emulsion of claim 82 or 83, wherein the lipid comprises glycerol/glyceryl linoleate (e.g., Maisine® CC).

85. The emulsion of any of claims 82-84, wherein the lipid further comprises propylene glycol monocaprylate (e.g., Capmul PG-8) or propylene glycol monolaurate (e.g., Capmul PG-12, or Lauroglycol™ 90).

86. The emulsion of any of claims 82-85, comprising two or more, such as two or three, non-ionic surfactants.

87. The emulsion of any of claims 82-86, wherein the non-ionic surfactant comprises macrogolglycerol hydroxystearate (e.g., Kolliphor RH 40) and/or polyglyceryl oleate (e.g., Plurol Oleique CC 497 (polyglyceryl-3 dioleate)).

88. The emulsion of claim 87, wherein the surfactant further comprises oleoyl polyoxyl-6 glycerides (e.g., Labrafil® M 1944 CS) and/or lauroyl polyoxyl-6 glycerides (e.g., Labrafil 2130).

89. An emulsion produced by mixing the pharmaceutical composition of any one of claims 1-37 with water.

90. An emulsion produced by administering the pharmaceutical composition of any one of claims 1-37 to a mammal.

91. A method of treating or preventing a disease or disorder in a subject in need thereof, the method comprising administering to the subject an effective amount of the emulsion according to any one of claims 82-90, wherein the disease or disorder is selected from a sex hormone-dependent benign or malignant disorder, an androgen receptor driven cancer, a syndrome due to androgen excess, and a syndrome due to glucocorticoid excess.
